(19) United States
(12) Patent Application Publication
(10) Pub. No.: US 2010/0173298 A1
(43) Pub. Date: Jul. 8, 2010

(54) HCV GENE

(76) Inventors: Kenichi Mori, Saitama (JP); Noboru Maki, Saitama (JP); Hiromi Fukai, Saitama (JP); Chiharu Ohue, Saitama (JP)

Correspondence Address: BIRCH STEWART KOLASCH & BIRCH, PO BOX 747, FALLS CHURCH, VA, 22040-0747, US

(21) Appl. No.: 12/597,692

(22) PCT Filed: Apr. 28, 2008
(86) PCT No.: PCT/JP2008/058215

§ 371 Date: Feb. 17, 2010

(30) Foreign Application Priority Data
Apr. 27, 2007 (JP) 2007-119667

(51) Int. Cl. C12Q 1/68; C07H 21/04; C07K 14/005; C07H 21/02; C12N 15/63; C12N 5/071; C12N 7/00

(52) U.S. Cl. 435/6; 536/23.72; 530/350; 536/23.1; 435/320.1; 435/325; 435/235.1

(57) ABSTRACT

The present invention provides a hepatitis C virus gene, a replicon RNA derived from the gene, a replicon-replicating cell into which the replicon RNA is introduced, and a method for screening a drug using the replicon-replicating cell. By introducing a replicon RNA comprising (A) a polynucleotide having the nucleic acid sequence shown in SEQ ID NO: 5; (B) a polynucleotide having the nucleic acid sequence shown in SEQ ID NO: 7; (C) a polynucleotide coding for a polypeptide having the amino acid sequence shown in SEQ ID NO: 6; (D) a polynucleotide coding for a polypeptide having the amino acid sequence shown in SEQ ID NO: 8; or (E) a polynucleotide having a nucleic acid sequence having a homology of not less than 90% with the nucleic acid sequence shown in SEQ ID NO: 5 or SEQ ID NO: 7; or a replicon RNA of the genotype 1b comprising nucleotides coding for the 1804th amino acid leucine and the 1966th amino acid lysine in the amino acid sequence of a hepatitis C virus polyprotein and a polynucleotide coding for an NS4B protein, into a cell, the replicon-replicating cell can be prepared. By using the replicon-replicating cell, the screening for the drug can be carried out.

### FIELD OF THE INVENTION

The present invention relates to a hepatitis C virus (hereinafter also referred to “HCV”) gene, a replicon RNA derived from the gene, a replicon-replicating cell into which the replicon RNA is introduced, and a method for screening a drug using the replicon-replicating cell.

### BACKGROUND ART

HCV is a causal factor for chronic hepatitis C. According to statistics from WHO, it is estimated that 170 million people are infected in the world. HCV is a virus classified in the genus Flavivirus in the family Flaviviridae. It is thought to infect via blood or blood components, and to proliferate in the liver. Although those who are infected with HCV cause relatively benign symptoms in the early stages of infection, it develops into a chronic disease at a high frequency. After an asymptomatic period for a certain period of time, it develops into chronic hepatitis. Furthermore, as the period of infection prolongs, conditions of disease deteriorates to liver cirrhosis and eventually to liver cancer at a high frequency. It is thought that the hepatitis virus relates to 95% of the liver cancer and 80% of them are caused by HCV infection.

For a treatment of chronic hepatitis C, interferon is widely used. Recently, by improvement of pharmaceutical formulations of interferon, as well as improvement of administration methods such as a combination therapy of interferon and ribavirin or the like, HCV is eliminated out of the body and a rate of sustained virological response (SVR) is gradually increasing. Yet the rate of SVR by the interferon administration is still about 50% and there are thought to be many HCVs exhibiting resistance against the interferon treatment. Accordingly, development of a drug having therapeutic effects on the interferon-resistant viruses is desired.

In the development of such a drug, a system for screening the drug is required. It has been reported that HCV was infected to cells derived from human or monkey cells and proliferated in vitro. But such a proliferation system showed a low efficiency of infection and low efficiency of proliferation, and thus cannot be used as the system screening the drug.

Recently, Wakita et al. isolated HCV genotype 2a gene from a patient with fulminant hepatitis C (Patent Literature 1). From this isolated JFH1 strain, a full-length RNA was synthesized in vitro. When the full-length RNA was introduced into cells derived from human hepatocarcinoma (Huh7 cells), a replicon RNA which autonomously replicates in the cells was able to be obtained. In addition, it was confirmed that infectious particles were released in the culture supernatant of the cells into which the replicon RNA was introduced (Non-patent Literature 1). Hence, a system for reinfection and proliferation can be constructed by introducing the replicon RNA of the JFH1 strain into the cells derived from human hepatocarcinoma (Huh7 cells) and culturing the obtained infectious particles again with the cells derived from human hepatocarcinomana. Using this system for reinfection and proliferation, screening for a drug against HCV has been started.

However, the JFH1 strain is an HCV of the genotype 2a and an interferon-sensitive HCV. For that, the JFH1 strain does not have an HCV gene region showing the interferon resistance. Also, a factor in a host, which factor acts on a region defining the interferon resistance, cannot be specified. Thus, there is a possibility that a drug effective against interferon-resistant HCV cannot be screened.

Recently, Lemon et al. reported a system for infection and proliferation in which a replicon RNA of H77 strain belonging to genotype 1a was introduced into the cells derived from human hepatocarcinoma (Huh7 cells) (Non-patent Literature 2). However, when virus particles obtained from the culture supernatant of the cells in which this replicon RNA was introduced were again infected to cells derived from human hepatocarcinoma, the infectivity titer was about 400 times lower, compared with that of the above-described JFH1 strain. Therefore, the replicon RNA of the H77 strain is thought to release virus particles which have lost infectivity. Consequently, it is thought that the RNA replicon of the H77 strain which is replicable in vitro has lost a mechanism to produce the infectious particles and does not retain a proliferation mechanism intrinsic to HCV.

Accordingly, there is a possibility that a system for screening using the system for infection and proliferation of this replicon RNA of H77 cannot screen an effective drug against HCV having a proliferation mechanism in vivo.

As described above, since there are no effective systems for culturing HCV in vitro, it has been difficult to carry out screening for a drug useful for the HCV treatment. For instance, as for interferon which is currently widely used for the HCV treatment, direct therapeutic methods have been developed and improved using patients as test subjects, which has been imposed a very heavy burden on the patients. Although the above-described replicon RNAs reported by Wakita and Lemon enabled some drugs to be screened, these replicon RNAs have problems described above. Hence, it is thought that a drug which can be widely used for the HCV treatment cannot be screened.

Patent Literature 1: Japanese Laid-open Patent Application (Kokai) No. 2002-171978

Non-patent Literature 1: “Nature Medicine” (U.S.A.) 2005, volume 11, p 791-796

Non-patent Literature 2: “Proceeding of the National Academy of Science of the United State of America” 2006, volume 103, p 2310-2315

### DISCLOSURE OF THE INVENTION

### Problems which the Invention Tries to Solve

The present inventors intensively studied, in order to obtain a drug which can be widely used for the HCV treatment, an effective system for proliferating HCV, in particular, an in vitro system for proliferating HCV having the genotype 1b gene, resistant to interferon and capable of producing infectious particles. First, a full-length of HCV genome was isolated from the serum of a patient with fulminant hepatitis and the nucleic acid sequence with 9594 bases shown in SEQ ID NO: 1 was determined. When a replicon RNA was prepared from this HCV gene and transfected into cells derived from human hepatocarcinoma, it was confirmed that the replicon RNA autonomously replicated in the cells and replication of RNA occurred. Further, by introducing two mutations of amino acid sequence in an NS4B protein region of this replicon RNA, replication efficiency of RNA markedly increased and lots of virus particles were released into the culture supernatant. And then, by culturing this HCV particles again with the cells derived from human hepatocarcinoma, construction of a system for reinfection and proliferation was possible. And, the present inventors found that this system for reinfection and proliferation using the replicon-replicating cells and infectious particles was useful as a system for screening a drug for the HCV treatment, in particular, a system for screening a drug for an interferon-resistant virus.

The present invention is based on such discoveries.

### Means for Solving the Problems

Accordingly, the present invention relates to a HCV gene comprising a polynucleotide selected from the group consisting of:

(A) a polynucleotide having the nucleic acid sequence shown in SEQ ID NO: 5;
(B) a polynucleotide having the nucleic acid sequence shown in SEQ ID NO: 7;
(C) a polynucleotide having the nucleic acid sequence shown in SEQ ID NO: 65;
(D) a polynucleotide coding for a polypeptide having the amino acid sequence shown in SEQ ID NO: 6;
(E) a polynucleotide coding for a polypeptide having the amino acid sequence shown in SEQ ID NO: 8; and
(F) a polynucleotide coding for a polypeptide having the amino acid sequence shown in SEQ ID NO: 66.

In a preferred mode of the HCV gene according to the present invention, the HCV gene is a polynucleotide having the nucleic acid sequence shown in SEQ ID NO: 1, SEQ ID NO: 3, SEQ ID NO: 10, SEQ ID NO: 61 or SEQ ID NO: 63.

Also, the present invention relates to a HCV genotype 1b gene comprising nucleotides coding for the 1804th amino acid leucine and the 1966th amino acid lysine in the amino acid sequence of a HCV polyprotein and a polynucleotide coding for an NS4B protein.

Additionally, the present invention also relates to a DNA comprising a single stranded DNA having the nucleic acid sequence with uridine being substituted by thymine in the nucleic acid sequence of the above-described HCV gene.

The present invention also relates to a polypeptide having the amino acid sequence shown in SEQ ID NO: 6, SEQ ID NO: 8 or SEQ ID NO: 66.

Further, the present invention also relates to a HCV polyprotein, wherein a peptide in an NS4B region is a polypeptide having the amino acid sequence shown in SEQ ID NO: 6, SEQ ID NO: 8 or SEQ ID NO: 66.

Also, the present invention also relates to at least one HCV protein selected from the group consisting of a core protein having the amino acid sequence from the first to the 191st amino acid in the amino acid sequence shown in SEQ ID NO: 2, SEQ ID NO: 4, SEQ ID NO: 11, SEQ ID NO: 62 or SEQ ID NO: 64, an E1 protein having the amino acid sequence from the 192nd to the 383rd amino acid, an E2 protein having the amino acid sequence from the 384th to the 746th amino acid, a P7 protein having the amino acid sequence from the 747th to the 809th amino acid, an NS2 protein having the amino acid sequence from the 810th to the 1026th amino acid, an NS3 protein having the amino acid sequence from the 1027th to 1657th amino acid, an NS4A protein having the amino acid sequence from the 1658th to 1711th amino acid, an NS4B protein having the amino acid sequence from the 1712th to the 1972nd amino acid, an NS5A protein having the amino acid sequence from the 1973rd to the 2419th amino acid, and an NS5B protein having the amino acid sequence from the 2420th to the 3010th amino acid.

The present invention also relates to a replicon RNA comprising a polynucleotide selected from the group consisting of:

(A) a polynucleotide having the nucleic acid sequence shown in SEQ ID NO: 5;
(B) a polynucleotide having the nucleic acid sequence shown in SEQ ID NO: 7;
(C) a polynucleotide having the nucleic acid sequence shown in SEQ ID NO: 65;
(D) a polynucleotide coding for a polypeptide having the amino acid sequence shown in SEQ ID NO: 6;
(E) a polynucleotide coding for a polypeptide having the amino acid sequence shown in SEQ ID NO: 8;
(F) a polynucleotide coding for a polypeptide having the amino acid sequence shown in SEQ ID NO: 66; and
(G) a polynucleotide having a nucleic acid sequence having a homology of not less than 90% with the nucleic acid sequence shown in SEQ ID NO: 5, SEQ ID NO: 7 or SEQ ID NO: 65.

Also, the present invention relates to a replicon RNA of the genotype 1b, the replicon RNA comprising nucleotides coding for the 1804th amino acid leucine and the 1966th amino acid lysine in the amino acid sequence of an HCV polyprotein and a polynucleotide coding for an NS4B protein.

In a preferred mode of the replicon RNA according to the present invention, the replicon RNA comprises:

(A) a polynucleotide from the first to the 341st nucleotide in the 5′ untranslated region of the HCV, a polynucleotide coding for a polypeptide from the 1027th to the 3010th amino acid in the HCV polyprotein and a polynucleotide of the 3′ untranslated region; or
(B) a polynucleotide from the first to the 341st nucleotide in the 5′ untranslated region, a polynucleotide coding for the HCV polyprotein composed of 3010 amino acids and a polynucleotide of the 3′ untranslated region.

In a preferred mode of the replicon RNA according to the present invention, the replicon RNA is resistant to interferon.

In another preferred mode of the replicon RNA according to the present invention, the replicon RNA comprises:

(A) a polynucleotide having the first to the 341st nucleotide in the nucleic acid sequence shown in SEQ ID NO: 1 and a polynucleotide having the 3420th to the 9594th nucleotide in the nucleic acid sequence shown in SEQ ID NO: 1;
(B) a polynucleotide having the first to the 341st nucleotide in the nucleic acid sequence shown in SEQ ID NO: 3 and a polynucleotide having the 3420th to the 9594th nucleotide in the nucleic acid sequence shown in SEQ ID NO: 3;
(C) a polynucleotide having the first to the 341st nucleotide in the nucleic acid sequence shown in SEQ ID NO: 10 and a polynucleotide having the 3420th to the 9594th nucleotide in the nucleic acid sequence shown in SEQ ID NO: 10;
(D) a polynucleotide having the first to the 341st nucleotide in the nucleic acid sequence shown in SEQ ID NO: 61 and a polynucleotide having the 3420th to the 9594th nucleotide in the nucleic acid sequence shown in SEQ ID NO: 61;
(E) a polynucleotide having the first to the 341st nucleotide in the nucleic acid sequence shown in SEQ ID NO: 63 and a polynucleotide having the 3420th to the 9594th nucleotide in the nucleic acid sequence shown in SEQ ID NO: 63;
(F) a polynucleotide having a nucleic acid sequence having a homology of not less than 90% with the nucleic acid sequence from the first to the 341st nucleotide in the nucleic acid sequence shown in SEQ ID NO: 1 and a polynucleotide having a nucleic acid sequence having a homology of not less than 90% with the nucleic acid sequence from the 3420th to the 9594th nucleotide in the nucleic acid sequence shown in SEQ ID NO: 1;
(G) a polynucleotide having a nucleic acid sequence having a homology of not less than 90% with the nucleic acid sequence from the first to the 341st nucleotide in the nucleic acid sequence shown in SEQ ID NO: 3 and the polynucleotide having a nucleic acid sequence having a homology of not less than 90% with the nucleic acid sequence from the 3420th to the 9594th nucleotide in the nucleic acid sequence shown in SEQ ID NO: 3;
(H) a polynucleotide having a nucleic acid sequence having a homology of not less than 90% with the nucleic acid sequence from the first to the 341st nucleotide in the nucleic acid sequence shown in SEQ ID NO: 10 and a polynucleotide having a nucleic acid sequence having a homology of not less than 90% with the nucleic acid sequence from the 3420th to the 9594th nucleotide in the nucleic acid sequence shown in SEQ ID NO: 10;
(I) a polynucleotide having a nucleic acid sequence having a homology of not less than 90% with the nucleic acid sequence from the first to the 341st nucleotide in the nucleic acid sequence shown in SEQ ID NO: 61 and a polynucleotide having a nucleic acid sequence having a homology of not less than 90% with the nucleic acid sequence from the 3420th to the 9594th nucleotide in the nucleic acid sequence shown in SEQ ID NO: 61; or
(J) a polynucleotide having a nucleic acid sequence having a homology of not less than 90% with the nucleic acid sequence from the first to the 341st nucleotide in the nucleic acid sequence shown in SEQ ID NO: 63 and a polynucleotide having a nucleic acid sequence having a homology of not less than 90% with the nucleic acid sequence from the 3420th to the 9594th nucleotide in the nucleic acid sequence shown in SEQ ID NO: 63.

Also, in another preferred mode of the replicon RNA according to the present invention, the above-described replicon RNA is a polynucleotide having the nucleic acid sequence shown in SEQ ID NO: 1, SEQ ID NO: 3, SEQ ID NO: 10, SEQ ID NO: 61 or SEQ ID NO: 63; or a polynucleotide having a nucleic acid sequence having a homology of not less than 90% with the nucleic acid sequence shown in SEQ ID NO: 1, SEQ ID NO 3, SEQ ID NO: 10, SEQ ID NO: 61 or SEQ ID NO: 63. Further, in another preferred mode of the replicon RNA according to the present invention, the replicon RNA comprises at least one selection marker gene or reporter gene and at least one IRES sequence.

Further, the present invention relates to a DNA coding for the above-described replicon RNA.

Further, the present invention relates to a vector comprising the above-described DNA.

Also, the present invention relates to a replicon-replicating cell made by introducing at least one selected from the group consisting of the above-described replicon RNA, the above-described DNA and the above-described vector into a cell.

In a preferred mode of the replicon-replicating cell according to the present invention, the above-described cell is a cell derived from a hepatocyte. Preferably the above-described cell derived from the hepatocyte is a Huh-7 cell.

The present invention relates to a replicon RNA produced by the above-described replicon-replicating cell.

Further, the present invention relates to at least one HCV protein selected from the group consisting of CORE, E1, E2, P7, NS2, NS3, NS4A, NS4B, NS5A and NS5B produced by the above-described replicon-replicating cell.

Further, the present invention relates to a HCV particle produced by the above-described replicon-replicating cell.

Also, the present invention relates to a method for screening a substance controlling HCV infection, which method comprises the steps of contacting the above-described replicon-replicating cell with the above-described substance and analyzing a degree of increase in the replicon RNA.

In a preferred mode of the method for screening according to the present invention, the above-described analysis of the degree of increase in the replicon RNA is detection of the replicon RNA or HCV protein.

In the present specification, “replicon RNA” means RNA which is produced based on viral RNA and has an ability to autonomously replicate in cells. As long as it can cause RNA replication, the replicon RNA includes ones capable and incapable of generating a virus particle.

Also, in the present specification, “interferon resistance” means that replication or proliferation of HCV is not significantly suppressed by administration of interferon in vitro and in vivo.

### EFFECTS OF THE INVENTION

The HCV gene according to the present invention allows an HCV gene capable of replicating in vivo to be analyzed in vitro. By using this HCV gene, the RNA replicon according to the present invention can be produced. Further, by the replicon-replicating cells in which the above-described RNA replicon is introduced, screening of a drug against HCV is possible. The replicon RNA produced from the HCV gene according to the present invention is an interferon-resistant RNA replicon of the genotype 1b. In addition, it is possible that the replicon-replicating cells into which this replicon RNA is introduced produce the infectious virus particles. Therefore, it is possible to provide an in vitro model with the same proliferation mechanisms of HCV, which is the genotype 1b and resistant to interferon, as in vivo proliferation mechanisms. And this proliferation model of HCV enables a drug for suppressing aggravation of hepatitis to be screened and a pharmaceutical to be developed.

### BRIEF DESCRIPTION OF THE DRAWINGS

FIG. 1 shows the production of the core protein by introducing the replicon RNA according to the present invention into cells. pTPF1 and pTPF1/4B were introduced into Huh-7 cells by electroporation and 4, 24, 48 and 72 hours later, the concentration of the core protein in the culture supernatant was measured.

FIG. 2 shows reinfection of the infective particles produced from the replicon-replicating cells according to the present invention to cells. At 4, 24, 48, 72 and 96 hours after the infection, the concentration of the core protein in the culture supernatant was measured.

FIG. 3 shows inhibitory effects of cyclosporin A on the production of the core protein using the method for screening according to the present invention. The replicon RNA was introduced into cells with or without adding cyclosporin A, and 4, 24, 48, 72 and 96 hours later, the concentration of the core protein in the culture supernatant was measured.

FIG. 4 shows the production of the core protein by introducing the replicon RNA according to the present invention into cells. pAHC1 and pAHC/4Bm were introduced into Huh-7 cells by electroporation, and 4, 24, 48 and 72 hours later, the concentration of the core protein in the culture supernatant was measured.

### BEST MODE FOR CARRYING OUT THE INVENTION

The best mode of the present invention will be described below but the present invention is by no means limited to this mode.

As long as the HCV gene according to the present invention is an HCV gene belonging to the genotype 1b it is not restricted. Preferably, the gene contains a polynucleotide coding for an NS4B protein according to the present invention. Also, the gene is preferred to be an HCV gene exhibiting interferon resistance.

The HCV gene can be classified into at least six types of the genotypes based on its nucleic acid sequence. Among them, HCV belonging to the genotype 1 is further classified into genotype 1a and genotype 1b. Specifically, the genotype of genotype 1b includes an HCV having a polynucleotide having a nucleic acid sequence having a homology of not less than 90% with the nucleic acid sequence shown in SEQ ID NO: 5 or 7.

Examples of the NS4B protein according to the present invention include a polypeptide having the amino acid sequence shown in SEQ ID NO: 6, SEQ ID NO: 8 or SEQ ID NO: 66. The HCV gene is composed of a region coding for a core protein, E1 protein and E2 protein, which are viral structural proteins; and a P7 protein, NS2 protein, NS3 protein, NS4A protein, NS4B protein, NS5A protein and NS5B protein, which are non-structural proteins, between the 5′ untranslated region (5′ UTR) and 3′ untranslated region (3′ UTR). The HCV gene, after infecting, functions as mRNA in host cells and a polyprotein with a length of about 3000 consecutive amino acids is synthesized. Thereafter, it is cleaved by a signal peptidase and signal peptidyl peptidase of the host cells as well as a protease encoded by the HCV genome, to yield the above-described three types of the structural proteins and seven types of the non-structural proteins.

Among these ten types of the HCV proteins, the NS4B protein forms a complex with other non-structural proteins from NS3 to NS5B, which complex forms an RNA replicating complex with proteins of the host cells. The RNA replicating complex is thought to replicate the genome RNA and plays an important role in viral replication. A polypeptide having the amino acid sequence shown in SEQ ID NO: 6 according to the present invention (hereinafter referred to as “TPF1-NS4B polypeptide”) which is encoded by the NS4B region of the HCV gene obtained from patients with fulminant hepatitis, a polypeptide having the amino acid sequence shown in SEQ ID NO: 8 according to the present invention (hereinafter referred to as “TPF1-mutated NS4B polypeptide”), and a polypeptide having the amino acid sequence shown in SEQ ID NO: 66 according to the present invention (hereinafter also referred to as AHC1-mutated NS4B polypeptide), in particular the TPF1-mutated NS4B polypeptide and AHC1-mutated NS4B polypeptide exhibit prominent effects on the replication of the HCV gene.

Therefore, the HCV gene according to the present invention is an HCV gene preferably comprising a polynucleotide coding for TPF1-NS4B polypeptide or TPF1-mutated NS4B polypeptide, more preferably a polynucleotide having the nucleic acid sequence shown in SEQ ID NO:5 (herein after referred to as TPF1-NS4B polynucleotide), most preferably a polynucleotide having the nucleic acid sequence shown in SEQ ID NO:7 (TPF1-mutated NS4B polynucleotide) or a polynucleotide having the nucleic acid sequence shown in SEQ ID NO:65 (AHC1-mutated NS4B polynucleotide). However, the HCV gene according to the present invention is not restricted as long as it exhibits the prominent effects on the replication of the HCV gene. For instance, the HCV gene according to the present invention can be a HCV gene having a homology of preferably not less than 90%, more preferably not less than 95%, still more preferably not less than 97%, still more preferably not less than 99%, with the nucleic acid sequence shown in SEQ ID NO: 5, SEQ ID NO: 7 or SEQ ID NO: 65.

As long as the HCV gene according to the present invention contains a polynucleotide in an NS4B region, it is not restricted, and includes a partial HCV gene containing a partial polynucleotide of HCV and HCV gene having the full-length HCV polynucleotide.

Examples of the partial polynucleotide include nucleotides having an arbitrary nucleic acid sequence region in SEQ ID NO: 1, 3, 10,61 or 63, and, in particular, partial nucleotides of 5′UTR (nucleic acid sequence from the first to the 341st nucleotide), core (nucleic acid sequence from the 342nd to the 914th nucleotide), E1 region (nucleic acid sequence from the 915th to the 1490th nucleotide), E2 region (nucleic acid sequence from the 1491st to the 2579th nucleotide), P7 region (nucleic acid sequence from the 2580th to the 2768th nucleotide), NS2 region (nucleic acid sequence from the 2769th to the 3419th nucleotide), NS3 region (nucleic acid sequence from the 3420th to the 5312th nucleotide), NS4A region (nucleic acid sequence from the 5313th to the 5474th nucleotide), NS4B region (nucleic acid sequence from the 5475th to the 6257th nucleotide), NS5A region (nucleic acid sequence from the 6258th to the 7598th nucleotide), NS5B region (nucleic acid sequence from the 7599th to the 9371st nucleotide) and 3′ untranslated region (nucleic acid sequence from the 9372nd to the 9594th nucleotide). Also, examples of the HCV gene having the full-length HCV polynucleotide include HCV genes having the nucleic acid sequence shown in SEQ ID NO: 1, SEQ ID NO: 3, SEQ ID NO: 10, SEQ ID NO: 61, or SEQ ID NO: 63.

The HCV gene according to the present invention includes a gene isolated from a patient with fulminant hepatitis. Among hepatitis, fulminant hepatitis means one which develops the second-degree encephalopathy or more severe encephalopathy and a prothrombin time of not more than 40% within eight weeks after the onset of the disease. It divided into acute hepatitis C type which develops encephalopathy within ten days and subacute hepatitis C type in which encephalopathy appears after ten or more days.

Cloning of the HCV gene according to the present invention can, for example, be carried out as follows. Total RNA is prepared from the serum of a patient with fulminant hepatitis C using acidity guanidine isothiocyanate.phenol.chloroform method (for example, ISOGEN-LS, manufactured by Nippon Gene) or the like. cDNA is synthesized from the total RNA by a reverse transcription reaction using a 3′ UTR specific primer and mouse leukemia virus reverse transcriptase (Superscript II, manufactured by Life technologies).

The synthesized HCV cDNA is amplified by PCR using specific primers from 5′ UTR to 3′ UTR (PCR Protocols, Academic Press (1990)). The amplified HCV cDNA is cloned into a pGEM-T EASY vector (manufactured by Promega) to determine the nucleic acid sequence.

Both termini of the HCV gene can be obtained by 5′-RACE using a 5′ UTR specific primer and 3′-RACE using a 3′ UTR specific primer (Proc. Natl. Acad. Sci. USA, 85, 8998 (1988)). The obtained cDNA fragments can be ligated together to obtain the full-length HCV genome.

The DNA according to the present invention is not restricted as long as it is a DNA corresponding to the above-described HCV gene which is RNA. An example includes double stranded DNA composed of a single strand cDNA synthesized with a reverse transcriptase from the HCV gene and a complementary strand of the single strand cDNA.

As long as the polypeptide according to the present invention is a polypeptide encoded by the polynucleotide having the nucleic acid sequence shown in SEQ ID NO: 1, 3, 10, 61, or 63, or a polypeptide containing the 1804th leucine and the 1966th lysine in the amino acid sequence of the HCV polyprotein, its region and length are not restricted. Preferably it is a polypeptide having the amino acid sequence shown in SEQ ID NO:6, SEQ ID NO:8, or SEQ ID NO:66.

The HCV protein according to the present invention includes a core protein having the amino acid sequence from the first to the 191st amino acid in the amino acid sequence shown in SEQ ID NO: 2, 4, 11, 62 or 64, an E1 protein having the amino acid sequence from the 192nd to the 383rd amino acid, an E2 protein having the amino acid sequence from the 384th to the 746th amino acid, a P7 protein having the amino acid sequence from the 747th to the 809th amino acid, an NS2 protein having the amino acid sequence from the 810th to the 1026th amino acid, an NS3 protein having the amino acid sequence from the 1027th to 1657th amino acid, an NS4A protein having the amino acid sequence from the 1658th to 1711th amino acid, an NS4B protein having the amino acid sequence from the 1712th to the 1972nd amino acid, an NS5A protein having the amino acid sequence from the 1973rd to the 2419th amino acid, or an NS5B protein having the amino acid sequence from the 2420th to the 3010th amino acid.

As long as the replicon RNA according to the present invention is RNA containing a polynucleotide having the nucleic acid sequence of the genotype 1b and having an ability to autonomously replicate in cells, it is not restricted. Examples of the nucleotide region involving in the replication of the HCV replicon RNA especially include the nucleotide regions coding for the 5′ UTR, 3′ UTR, and nonstructural proteins such as an NS3 protein, NS4A protein, NS4B protein, NS5A protein, and NS5B protein. In the replicon RNA according to the present invention, all of these regions are important but the region coding for the NS4B protein is important in terms of increasing replication efficiency. In particular, the NS4B protein is preferably the TPF1-NS4B polypeptide which is a polypeptide having the amino acid sequence shown in SEQ ID NO:6, more preferably the TPF1-mutated NS4B polypeptide which is a polypeptide having the amino acid sequence shown in SEQ ID NO:8, or the AHC1-mutated NS4B polypeptide which is a polypeptide having the amino acid sequence shown in SEQ ID NO:66.

Therefore, the replicon RNA according to the present invention is preferably a replicon RNA containing a polynucleotide coding for the TPF1-NS4B polypeptide, in particular a polynucleotide having the nucleic acid sequence shown in SEQ ID NO: 5 (TPF1-NS4B polynucleotide), more preferably a replicon RNA containing a polynucleotide coding for the TPF1-mutated NS4B polypeptide, in particular a polynucleotide having the nucleic acid sequence shown in SEQ ID NO: 7 (TPF1-NS4B mutated polynucleotide) or a polynucleotide coding for the AHC1-mutated NS4B polypeptide, in particular a polynucleotide having the nucleic acid sequence shown in SEQ ID NO: 65 (AHC1-mutated NS4B polynucleotide). However, the replicon RNA according to the present invention is not restricted to the above-described replicon RNA containing the polynucleotide of the NS4B region and includes a replicon RNA containing a polynucleotide having a nucleic acid sequence having a homology of preferably not less than 90%, more preferably not less than 95%, still more preferably not less than 97%, most preferably not less than 99%, with the nucleic acid sequence shown in SEQ ID NO:5, SEQ ID NO:7 or SEQ ID NO:65.

The TPF1-mutated NS4B polynucleotide (SEQ ID NO: 7) is a polynucleotide wherein the 278th nucleotide A is substituted with U and the 763rd nucleotide G is substituted with A in the TPF1-NS4B polynucleotide (SEQ ID NO: 5). The TPF1-mutated NS4B polypeptide (SEQ ID NO: 8) is a polypeptide wherein the 93rd amino acid glutamine (Q) is substituted with leucine (L) and the 255th amino acid glutamic acid (E) is substituted with lysine (K) in the TPF1-NS4B polypeptide (SEQ ID NO: 6).

In a mode of the HCV gene and replicon RNA according to the present invention, nucleotides coding for the 1804th amino acid leucine and the 1966th amino acid lysine in the amino acid sequence of the HCV polyprotein can be included.

The positions of the 1804th amino acid leucine and the 1966th amino acid lysine are positions in HCV genotype 1b gene composed of 3010 amino acids.

The 1804th amino acid leucine and the 1966th amino acid lysine are amino acids contained in the NS4B protein. Thus far, an NS4B protein containing these amino acids has not been reported. Hence, an HCV polyprotein containing these amino acids and RNA replicon containing a polynucleotide coding for these amino acids have not been reported.

Although a nucleic acid sequence of the HCV genotype 1b gene is not particularly restricted, it includes, for example, a nucleic acid sequence having a homology of not less than 90% with the nucleic acid sequence shown in SEQ ID NO:1, SEQ ID NO:3, SEQ ID NO:10, SEQ ID NO:61, or SEQ ID NO:63.

As long as the replicon RNA according to the present invention can replicate in cells, its structure is not restricted. Examples include those containing the full-length HCV RNA and a subgenomic replicon RNA containing a part of the RNA. For instance, the subgenomic replicon RNA can contain a nucleotide region coding for the 5′ untranslated region (hereinafter also referred to as 5′ UTR), the 3′ untranslated region (hereinafter also referred to as 3′ UTR), and the NS3 protein, NS4A protein, NS4B protein, NS5A protein and NS5B protein, which are nonstructural proteins, preferably a polynucleotide having the nucleic acid sequence from the first to the 341st nucleotide in the nucleic acid sequence shown in SEQ ID NO:1, SEQ ID NO:3, SEQ ID NO:61 or SEQ ID NO:63; and a polynucleotide having the nucleic acid sequence from 3420th to the 9564th nucleotide in the nucleic acid sequence shown in SEQ ID NO:1, SEQ ID NO:3, SEQ ID NO:61 or SEQ ID NO:63.

5′ UTR is usually composed of 341 nucleotides in the HCV genotype 1b gene. Although the nucleic acid sequence of 5′ UTR contained in the replicon RNA is not restricted, it preferably contains its entire length of the sequence. The length of the 3′ UTR varies depending on the viral strain. 3′ UTR is usually composed of variable region with 41 nucleotides, a poly U region whose length varies depending on the strain and 3′ X region with 98 nucleotides. Although the nucleic acid sequence and length of 3′UTR contained in the replicon RNA is not restricted, it preferably contains the entire length of 3′UTR in the strain.

Also, in addition to the full-length RNA and subgenomic RNA, a selection marker gene, reporter gene, or IRES sequence may be contained. Examples of such a replicon RNA include a replicon RNA having the polynucleotide shown in SEQ ID NO:9, SEQ ID NO:67, or SEQ ID NO:68.

Examples of the selection marker include antibiotic resistance genes. Examples of the preferred selection marker genes in the present invention include neomycin resistance genes, thymidine kinase genes, kanamycin resistance genes, pyrithiamin resistance genes, adenylyl transferase genes, zeocin resistance genes and puromycin resistance genes. The neomycin resistance genes and thymidine kinase genes are preferred and the neomycin resistance genes are most preferred. However, the selection marker gene in the present invention is not limited thereto.

An example of the reporter gene includes structural genes of an enzyme which catalyzes a luminescent reaction and coloring reaction. Examples of the preferred reporter gene in the present invention include chloramphenicol acetyltransferase genes derived from transposon Tn9, β-glucuronidase or β-galactosidase genes derived from E. coli, luciferase genes, green fluorescent protein genes, aequorin genes derived from jellyfish and secreted placental alkaline phosphatase (SEAP) genes. However, the reporter gene according to the present invention is not limited thereto.

Examples of the IRES sequence include, but are not limited to, EMCV IRES (internal ribosome entry site of an encephalomyocarditis virus), FMDV IRES, HCV IRES and the like. EMCV IRES and HCV IRES are preferred and EMCV IRES is most preferred.

The replicon RNA according to the present invention is preferably resistant to interferon. When a patient with HCV is treated with interferon, whether interferon is effective or not is thought to depend on, for example, a factor attributed to the virus and a factor attributed to the host. The factor attributed to the virus includes an HCV gene region exhibiting the interferon resistance. The replicon RNA according to the present invention preferably contains the HCV gene area exhibiting the interferon resistance. The HCV gene area exhibiting the interferon resistance is not particularly restricted and can be, for example, an ISDR region which is thought be an index for IFN sensitivity in an NS5A region.

The replicon RNA according to the present invention is preferably an RNA replicon containing a polynucleotide having the nucleic acid sequence of the genotype 1b but includes, for example, a RNA replicon containing a polynucleotide having a nucleic acid sequence having a homology of not less than 90% with the nucleic acid sequence shown in SEQ ID NO:5, SEQ ID NO:7, or SEQ ID NO:65.

As long as the DNA according to the present invention is a form of linear DNA and DNA coding for the above-described replicon RNA, it is not restricted. It can, for example, contain an RNA promoter to generate a replicon RNA.

The replicon RNA according to the present invention can be produced using an arbitrary gene engineering technique. Although it is not restricted, the replicon RNA can, for example, be produced by the method below. A DNA coding for the above-described replicon RNA is inserted into a cloning vector by a conventional method to produce a DNA clone. This DNA is inserted downstream of an RNA promoter to produce a DNA clone capable of generating a replicon RNA. The above-described RNA promoter is preferably one contained in a plasmid clone. Examples of the RNA promoter include, but are not limited to, T7 RNA promoters, SP6 RNA promoters and SP3 RNA promoters with particularly the T7 RNA promoters being preferred.

A vector into which the DNA is to be inserted is not particularly restricted and examples thereof include plasmid vectors, linear double-stranded DNA vectors and virus vectors such as adenovirus vectors, adeno-associated virus vectors, retroviral vectors and lentivirus vectors. The plasmid vectors are preferred.

The replicon RNA according to the present invention can be produced from the above-described vector into which the DNA is inserted. The RNA is synthesized with the DNA clone as a template using an RNA polymerase. The RNA synthesis can be started from the 5′ untranslated region by a conventional method. In cases where a template DNA is a plasmid clone, the above-mentioned DNA region linked to the downstream of the RNA promoter can be excised from the plasmid clone using a restriction enzyme and the RNA is synthesized with the resulting DNA fragment as a template. The 3′ terminus of the synthesized RNA is preferably identical to the 3′ untranslated region of virus genome RNA. It is preferred that other sequence be not added or deleted. For instance, in the case of a preferred mode of the full-length replicon RNA according to the present invention, DNA is inserted into a vector having a T7 RNA promoter upstream of 5′UTR and a restriction enzyme Xba I site in the 3′UTR terminus. After digestion with Xba I, an HCV genome RNA can be synthesized using a T7 RNA polymerase.

The replicon-replicating cells according to the present invention can be prepared by introducing the above-described RNA replicon into arbitrary cells. Cells into which the replicon RNA is introduced are not particularly restricted and preferably are cells derived from human liver, cells derived from mouse liver or cells derived from monkey liver. Examples of the cells particularly include Huh7 cells, HepG2 cells, or Hep3B cells which are cells derived from human hepatocarcinoma, or IMY-N9 cells, HeLa cells, CHO cells, COS cells, Vero cells and 293 cells. The introduction of the replicon RNA into the cells can be carried out by an arbitrary transfection method. Examples of such an introduction method include electroporation, particle gun methods and lipofection methods. A method by the electroporation is especially preferred.

When a replicon RNA containing a selection marker gene or reporter gene for the introduction into cells is used, cells in which the replicon RNA is introduced and replicating continuously, can be selected using expression of the selection marker gene or reporter gene. For instance, in cases where a neomycin resistance gene is contained in the replicon RNA as the selection marker gene, cells into which the replicon RNA is transfected are plated in a culture dish, and G418 (neomycin) is added at a concentration of 0.05 mg/ml to 3.0 mg/ml. Thereafter, the medium is changed twice a week to continue the culture. At two to three weeks after plating, the resulting cells can be visualized as a colony.

The replicon-replicating cells according to the present invention produce a replicon RNA, HCV protein and HCV particle. Therefore, by using the replicon-replicating cells, the replicon RNA, HCV protein and HCV particle can be produced.

The replicon RNA which replicates in the replicon-replicating cells can be extracted from the cells using an arbitrary RNA extraction method. The RNA extracted from the cells can be made to function as a replicon RNA by being again introduced into the other cells. The HCV protein according to the present invention can be used one secreted in the cells or into a culture supernatant. The produced HCV protein can be extracted and purified using a known method. Also, as for the HCV particles produced by replicon-replicating cells, those secreted in the cells or into the culture supernatant can be used. The HCV protein and HCV particle according to the present invention can be used as a vaccine by adding modifications to the replicon RNA to modify the RNA, virus protein or virus particle and to weaken pathogenicity.

By using the above-described replicon-replicating cells, a substance to control the infection of HCV can be screened. “To control the infection of HCV” means for example to control (e.g. to promote or to suppress) the replication of HCV RNA and to control (e.g. to promote or to suppress) the translation from RNA to a protein. Concretely, by contacting the replicon-replicating cells with a test substance and analyzing the degree of increase of the replicon RNA, the screening of the test substance can be carried out. The degree of increase of the replicon RNA means a change of the replication rate or amount of the replicon RNA. Concretely, by detecting or measuring the amount of the replicon RNA in replicon-replicating cells, and comparing with the amount of the replicon RNA in the replicon replicating cells without contacting with the test substance, which cells are a control, the test substance can be screened. Also, by detecting or measuring the amount of the HCV protein in cells or the supernatant, and comparing with that in control replicon replicating cells which do not contact with the test substance, the test substance can be screened. The HCV protein which can be detected or measured in the screening is not particularly restricted and is preferably a core protein. The core protein can be measured using a commercially available kit. In addition, by automating the screening method, adaptation to a high-throughput screening method is possible.

Further, the screening method according to the present invention is useful as a method for evaluating effects of the screened drug. In cases where the evaluation of the drug needs to be carried out by this screening method, it can be used as a method for producing a drug.

### <<Action>>

The HCV gene and replicon RNA containing the nucleotides coding for the 1804th amino acid leucine and the 1966th amino acid lysine in the amino acid sequence of the HCV polyprotein are not completely clarified but can be speculated as follows. Yet, the present invention is by no means limited to the description below.

The present inventors repeated the operation in the Example 9 using the genotype 1b strain coding for other 3010 amino acids, instead of the AHC1 strain used in the Example 9 described later, and obtained the same results as in Example 9. As a subgenomic replicon having an adaptive mutation other than the NS4B protein, a replicon with the 5308th base T being mutated to C and the 1656th amino acid V (valine) in 3010 amino acids of the HCV polyprotein being mutated to A (alanine); and a replicon with, in addition to the above mutation, the 6846th base A being mutated to G and the 2169th amino acid T (tyrosine) in 3010 amino acids of the HCV polyprotein being mutated to A (alanine) were obtained.

From the experimental results obtained from the TPF1 strain, AHC1 strain and the above-described genotype 1b strain, the following can be considered. The replicon RNA of the genotype 1b containing the nucleotides coding for the 1804th leucine and the 1966th lysine increases in RNA replication efficiency, compared with the replicon RNA without these nucleotides. Thus, since the replicon RNA of HCV genotype 1b has these two adaptive mutations, the RNA replication efficiency increases.

By transfecting cells using the replicon RNA having the above-described two adaptive mutations in the above-described NS4B protein, the replicon-replicating cells can be obtained with certainty. The replicon RNA obtained from these replicon-replicating cells may be introduced with one or more other adaptive mutations in addition to the above-mentioned two adaptive mutations in the NS4B protein. Therefore, by introducing one or more other adaptive mutations in addition to the above-mentioned two adaptive mutations in the NS4B protein, the replication efficiency of the replicon RNA may increase.

The adaptive mutations other than two of the above-described adaptive mutations in the above-described NS4B protein include known adaptive mutations and unknown adaptive mutations. As the known adaptive mutations, mutations shown in Table 1 have been reported.

TABLE 1

| Region | Position of aa | aa |
|---|---|---|
| NS3 | 1115 | P→L |
| | 1133 | V→I |
| | 1202 | E→G |
| | 1261 | T→S |
| | 1268 | M→V |
| | 1280 | T→I |
| | 1283 | R→G |
| | 1287 | T→A |
| | 1304 | G→S |
| | 1383 | E→A |
| | 1452 | I→L |
| | 1577 | K→R |
| | 1609 | K→E |
| NS4A | 1691 | K→R |
| NS4B | 1846 | K→T |
| | 1897 | V→L/M/A |
| | 1963 | P→S |
| NS5A | Ins2041 | K |
| | 2043 | S→Y |
| | 2163 | E→G |
| | 2177 | D→N/G |
| | 2189 | R→G |
| | 2196 | P→S |
| | 2197 | S→P/C |
| | 2199 | A→T/D/S |
| | 2202 | del S |
| | 2204 | S→I/R |
| | 2330 | K→E |
| | 2371 | Q→R |
| | 2379 | A→V |
| | 2411 | E→K |
| NS5B | 2442 | I→V |
| | 2884 | R→G |
| | 2933 | Q→R |
| | 3004 | I→T |

### EXAMPLES

### Example 1

### Isolation and Analysis of Full-Length Fulminant Hepatitis C Virus Gene

(A) Extraction of RNA from Serum

RNA was purified from the serum drawn from a patient at the acute stage of fulminant hepatitis (250 μl) using High Pure Viral Nucleic Acid Kit (Roche diagnostics corporation) in accordance with the method recommended by the manufacturer.

(B) Synthesis of cDNA and Amplification of cDNA by PCR

To the purified RNA, was added XR58R primer to carry out a reverse transcription reaction at 42° C. for an hour using SuperSucript II reverse transcriptase (Invitrogen) in accordance with the method recommended by the manufacturer, thereby obtaining cDNA. To the obtained reaction solution, was added RNaseH (Invitrogen) and the mixture was allowed to react at 37° C. for 30 minutes, to digest RNA. This reaction solution was subjected to polymerase chain reaction (PCR) involving 30 rounds of a thermal cycle reaction using HC-LongA1 primer and 1b9405R primer with Takara LA Taq DNA polymerase (Takara Shuzo), the thermal cycle reaction being composed of 94° C. for 20 seconds and 68° C. for nine minutes, thereby amplifying cDNA. Further, an aliquot of the obtained reaction solution was subjected to PCR using HC85F and HC9302R primers to amplify HCV cDNA.

(C) Cloning of cDNA

The amplified DNA fragment was separated by electrophoresis using 0.7% agarose gel and the DNA fragment was collected using QIAquick gel purification kit (QIAGEN) in accordance with the method recommended by the manufacturer. The collected DNA fragment was subjected to a ligation reaction with pGEM-T easy vector (Promega) and DH5a strain was transformed by the resulting plasmid. Ampicillin-resistant transformants were selected and cultured using 2YT culture medium. The plasmid was purified from the cultured bacteria using Wizard Plus SV Miniprep DNA Purification System.

### (D) Determination of the Nucleic Acid Sequence.

The nucleic acid sequence of HCV cDNA was determined using a primer designed based on the sequence of the genotype 1b of HCV. Using CEQ DTCS Quick Start Kit (Beckman Coulter), a reaction was carried out in accordance with the method by the manufacturer and an analysis was carried out using CEQ2000 XL DNA analysis system (Software version 4.0.0, Beckman Coulter). The obtained data was analyzed using Sequencher (Version 4.1.2, Gene Codes Corporation). The obtained HCV clone was named pTPF 1-0193.

(E) Cloning of cDNA of 5′ Untranslated Region and Determination of Nucleic Acid Sequence Thereof

Further, from the RNA obtained in the step according to the above-described (A), cDNA of the terminus of 5′ untranslated region was obtained by 5′RACE method, which was carried out using a kit of 5′RACE System for Rapid Amplification of cDNA Ends, Version2.0 (Invitrogen) in accordance with the attached instructions. Chiba-as was used as the antisense primer for the cDNA synthesis. cDNA was synthesized using SuperScript II Reverse Transcriptase (Invitrogen). After purified with S.N.A.P column, cDNA was subjected to a TdT-tailing reaction and dCTP was then added to the resultant. With 5′RACE Abridged Anchor primer which came in the kit and KY78 primer, the first PCR was carried out using Takara LA Taq DNA polymerase (Takara Shuzo). Using an aliquot of this PCR product as a template, and with UTP primer which came in the kit and KM2 primer, the second PCR was carried out using Takara LA Taq DNA polymerase (Takara Shuzo), thereby obtaining a PCR product. This PCR product was cloned into the pGEM-T easy vector. The nucleic acid sequence was determined in accordance with the step according to the above-described (D). An HCV cDNA clone containing the nucleotide from the first to the 709th in the obtained SEQ ID NO: 1 was named pTPF1-0007.

(F) Cloning of cDNA of 3′ Untranslated Region and Determination of Nucleic Acid Sequence Thereof

From the RNA obtained in the step according to the above-described (A), cDNA of the terminus of 3′ untranslated region was obtained by 3′RACE method. First, to the RNA from the patient, was added Poly(A) using Poly(A) Tailing Kit (Ambion) in accordance with the attached instructions. The above-described steps (B) to (D) were repeated except that dT-Adp primer was used instead of the XR58R primer, 3UTR-1F and Adp primers were used as the primers for the first PCR, and XR58F and Adp primers were used as the primers for the second PCR. The obtained HCV cDNA clone was named pTPF1-8994.

The obtained HCV strain was named TPF1 strain. The TPF1 strain is an HCV with the full-length of 9594 bases and the nucleic acid sequence is shown in SEQ ID NO: 1. The polynucleotide of the obtained TPF1 strain had a translation region coding for consecutive 3010 amino acids between the 342th and the 9374th base. The amino acid sequence of the polyprotein of the TPF1 strain is shown in SEQ ID NO: 2.

The primers used for cloning and determining the nucleic acid sequence are shown below.

XR58R (SEQ ID NO: 12):
5′-tcatgcggct cacggacctt tcacagctag-3′
HClongA1 (SEQ ID NO: 13):
5′-atcgtcttca cgcagaaagc gtctagccat-3′
1b9405R (SEQ ID NO: 14):
5′-gcctattggc ctggagtgtt tagctc-3′
HC85F (SEQ ID NO: 15):
5′-atggcgttag tatgagtgtc gtgcagcct-3′
HC9302R (SEQ ID NO: 16):
5′-tcgggcacga gacaggctgt gatatatgtc t-3′
chiba-as (SEQ ID NO: 17):
5′-tgcacggtct acgagacct-3′
KY78 (SEQ ID NO: 18):
5′-ctcgcaagca ccctatcagc cagt-3′
KM2 (SEQ ID NO: 19):
5′-aggcattgag cgggtttat-3′
dT-Adp (SEQ ID NO: 20):
5′-ctagactcga gtcgacatcg tttttttttt tttttttt-3′
3UTR-1F (SEQ ID NO: 21):
5′-atcttagccc tagtcacggc-3′
Adp (SEQ ID NO: 22):
5′-ctagactcga gtcgacatcg-3′
XR58F (SEQ ID NO: 23):
5′-ctagctgtaa aggtccgtga gccgcatga-3′
M13 Primer M3 (SEQ ID NO: 24):
5′-gtaaaacgac ggccagt-3′
M13 Primer RV (SEQ ID NO: 25):
5′-caggaaacag ctatgac-3′
104 (SEQ ID NO: 26):
5′-aggaagactt ccgagcggtc-3′
HC841S (SEQ ID NO: 27):
5′-ggaacttgcc cggttgctct ttctctatct tc-3′
E1 (SEQ ID NO: 28):
5′-attccatggt ggggaactgg gctaa-3′
HC2069S (SEQ ID NO: 29):
5′-taacaatacc ttgacctgcc ccacggactg-3′
HC2430S (SEQ ID NO: 30):
5′-aacatcgtgg acgtgcaata cctgtacgg-3′
HC2461AS (SEQ ID NO: 31):
5′-gaccctacac cgtacaggta-3′
HC2769S (SEQ ID NO: 32):
5′-ttggaccggg agatggctgc atcgtg-3′
HC3632F (SEQ ID NO: 33):
5′-cacccaaatg tacaccaatg t-3′
HC3928S (SEQ ID NO: 34):
5′-tacccgttga gtctatggaa ac-3′
HC4016AS (SEQ ID NO: 35):
5′-cacttggaat gtctgcggta-3′
HC4498S (SEQ ID NO: 36):
5′-agggggggag gcatctcatt ttctg-3′
HC4888F (SEQ ID NO: 37):
5′-tgctatgacg cgggctgtgc ttggta-3′
HC5381F (SEQ ID NO: 38):
5′-ggtcattgtg ggcaggatcat-3′
HC5692S (SEQ ID NO: 39):
5′-ctgcctggaa accccgcgat-3′
HC5858F (SEQ ID NO: 40):
5′-tggcagcata ggccttggga aggt-3′
HC631SF (SEQ ID NO: 41):
5′-aagacctggc tccagtccaa g-3′
5A-1 (SEQ ID NO: 42):
5′-ttccatgctc accgacccct c-3′
HC7090S (SEQ ID NO: 43):
5′-gtggagtcag agaataaggt-3′
HC7743F (SEQ ID NO: 44):
5′-cagaagaagg tcacctttgac-3′
HC8192S (SEQ ID NO: 45):
5′-gcagcgggtc gagttcctgg tgaat-3′
HC8939F (SEQ ID NO: 46):
5′-ctacggggcc tgttactcca ttgaac-3′

### Example 2

### Preparation of Subgenomic RNA Replicon

The full-length of the polynucleotide of the hepatitis C virus TPF1 strain was inserted into downstream of a T7 RNA promoter sequence in pBluescriptIISK(+) (hereinafter referred to as pTPF1).

Next, a region coding for a structural protein of pTPF1 and a part of a region coding for a nonstructural protein were replaced with a neomycin resistance gene (neomycin phosphotransferase, NPT-II) and EMCV-IRES (internal ribosome entry site of an encephalomyocarditis virus), thereby constructing plasmid DNA pRepTPF1. This construction procedure was carried out in accordance with a reported method (Lohmann et al., Science, (1999) 285, p. 110-113).

Specifically, pTPF1 was first digested with restriction enzymes, Age I and Bsr GI. To the resulting cleavage site, a PCR-amplified fragment from 5′UTR to the core region derived from pTPF1 and the neomycin resistance gene derived from pcDNA3.1(+), which fragment was digested with restriction enzymes, Age I and Pme I, and a PCR-amplified fragment from EMCV-IRES to NS3 region, which fragment was digested with restriction enzymes, Pme I and Bsr GI, were inserted and ligated. RNA was synthesized with this plasmid DNA pRepTPF 1 digested with Xba I as a template using Megascript T7 kit (Ambion). The RNA was purified in accordance with the method recommended by the manufacturer.

Human hepatocarcinoma cells (Huh7, JCRB0403) were cultured in Dulbecco's modified Eagle medium (D-MEM, IWAKI) containing 10% fetal bovine serum (FBS) with penicillin and streptomycin (50 U/mL and 50 μg/mL, respectively), under 5% CO2 conditions at 37° C. The cells before confluency were detached from the culture dish using a trypsin-EDTA treatment and resuspended in medium containing serum to inactivate trypsin. After washing twice with PBS, the cells were resuspended in Cytomix (120 mM Potassium chloride, 10 mM Potassium phosphate, 5 mM Magnesium chloride, 25 mM HEPES, 0.15 mM Calcium chloride, 2 mM EGTA, pH 7.6) and 1.25% DMSO was added, followed by transferring the mixture into an electroporation cuvette with a gap of 0.4 cm.

An appropriate amount of RNA is added to the cells and the mixture is then sufficiently cooled on ice for five minutes. A pulse is applied at 960 uF and 250 V using an electroporator (Bio-Rad). Immediately, the cells were resuspended in 8 ml of medium and a part of them is spread on a plate. After incubation for a certain period of time, G418 (neomycin) was added to the culture plate at a concentration of 1 mg/ml. Thereafter, the culture was continued with the culture medium being changed every four days. A colony of surviving cells was cloned from the culture plate about 20 days after plating, and the culture was continued. Such cloning of the colony made it possible to establish cells in which pRepTPF1 replicon RNA autonomously replicates. Whether or not the replicon RNA replicates was analyzed by a quantitative RT-PCR method measuring the copy number of replicating replicon RNAs contained in cellular RNAs.

### Method for Quantifying Minus Strand

Whether or not the autonomous replication of the replicon RNA took place was checked if the minus strand of the 5′UTR region of HCV RNA in the cells can be detected or not. The method for specifically quantifying the minus strand was carried out in the same manner as the method for specifically detecting the minus strand RNA, which method is described in Japanese Patent Application No. 08-187097.

The statistically significant amount of the minus strand was detected from the cells into which RNA was introduced by electroporation, the RNA being synthesized in vitro using pRepTPF-1 as a template. Thus it was confirmed that the replicon RNA autonomously replicated in the cells.

### Example 3

### Analysis of Adaptive Mutations

Intracellular RNAs were extracted from the replicon RNA-replicating cell line using ISOGEN (Nippon Gene) in accordance with the condition recommended by the manufacturer, which cell line was established by transfecting RNA synthesized in vitro with pRepTPF1 as a template into Huh7 cells in accordance with Example 2.

DNA for the almost entire region of the replicon RNA was amplified from this intracellular RNA in the same manner of obtaining the gene from TPF1 as described in Example 1. Specifically, cDNA corresponding to the replicon RNA was synthesized with the extracted intracellular RNA as a template usingSuperSucript II reverse transcriptase (Invitrogen) and XR58R primer.

Using an aliquot of this cDNA, a polymerase chain reaction (PCR) was carried out in the presence of EMCV-S1 primer: 5′-tgcacatgct ctacatgtgt ttagtcgagg-3′ (SEQ ID NO: 60) and HC9405R primer, using Takara LA Taq DNA polymerase (Takara Shuzo), which PCR involved 30 rounds of a thermal cycle reaction composed of 94° C. for 20 seconds and 68° C. for 6 minutes, thereby amplifying cDNA. When the nucleic acid sequence of the clone cloned into the pGEM-T easy vector was determined, it was found that the 5752nd base A was substituted with T and the 6237th base G was substituted with A. Consequently, Q (glutamine) at the amino acid corresponding to the amino acid number 1804 in SEQ ID NO: 2 was mutated to L (leucine) and E (glutamic acid) at the amino acid corresponding to the amino acid number 1966 was mutated to K (lysine).

Next, effects of the above-described amino acid substitution on the replication of the replicon RNA were examined. First, the adaptive mutations at the amino acid number 1804 (from Q to L) and at the amino acid number 1906 (from E to K) were introduced into the HCV RNA replicon pRepTPF1 prepared in Example 2 using, Quick Mutagenesis Kit (Stratagene) in accordance with the method recommended by the manufacturer. A replicon RNA in which this amino acid substitution was introduced was named pRep4B.

RNA was synthesized using pRepTPF1 which did not have a nucleic acid sequence causing the mutations and pRep4B having the amino acid mutations, both of which plasmid DNAs were cleaved with Xba I, as templates using Megascript T7 kit (Ambion). RNA was purified in accordance with the method recommended by the manufacturer. Each of the purified RNAs was transfected into Huh7 cells. The cells were cultured for about 20 days in the presence of G418 and surviving cells were stained with crystal violet. The number of the stained cells was measured to calculate the number of colony per 1 μg of the amount of the replicon RNA transfected.

When 1 μg of RepTPF1 RNA was transfected, one G418 resistant colony was selected whereas 10⁴ colonies were selected when 1 μg of Rep4B RNA was transfected. Hence, the nucleic acid mutations which caused the amino acid mutations in the replicon were thought to be adaptive mutations which increased the replication efficiency of the replicon RNA in Huh7 cells.

### Example 4

### Effects of Adaptive Mutations on HCV RNA Replication

The full-length HCV DNA pTPF1 prepared in Example 2 was digested with a restriction enzyme, Sfi I. A fragment obtained by digesting pRep4B with the restriction enzyme, Sfi I was inserted and ligated to the region of the cleavage, thereby preparing a full-length HCV DNA pTPF1/4B in which the adaptive mutations were inserted.

The replication efficiency of a full-length HCV RNA synthesized from pTPF1/4B in which the adaptive mutations were inserted was compared with the case of pTPF1. Specifically, the same method as described in Example 2 was carried out. The full length HCV RNA was synthesized in vitro and transfected into Huh7 cells. The transfected cells were immediately resuspended in 10 ml of the culture medium and plated 1 ml each in a 12-well plate (diameter 22.1 mm) to start a culture. Four hours, 24 hours, 48 hours and 72 hours later, culture supernatant was collected. The collected culture supernatant was centrifuged at 2 k rpm for 10 minutes and supernatant was collected. The supernatant (100 μl) was measured using a kit for HCV core antigen (FUJIREBIO, Lumipulse).

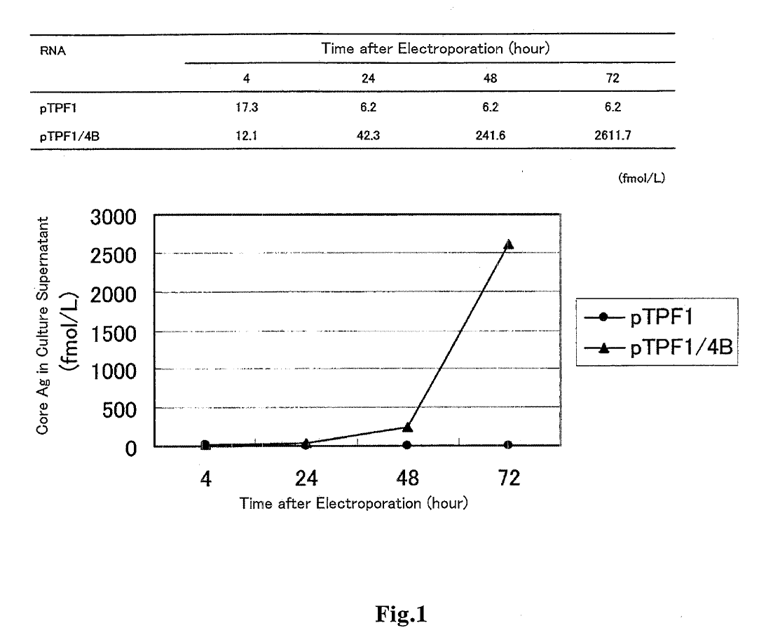

As shown in FIG. 1, the measured value of the core antigen in the supernatant of the pTPF1/4B in which the adaptive mutations were introduced was higher at any point, compared with the case of the pTPF1 which did not have the adaptive mutations and served as a control. This indicates that, by introducing the adaptive mutations according to the present invention into the full-length HCV RNA replicon, replication occurs at a high efficiency in cells and the core protein is secreted into the supernatant. It indicates that a full-length genome similar to the structure replicating in the liver is replicable in vitro.

In particular, it is thought that, in the TPF1-NS4B polypeptide according to the present invention, by using the polynucleotide coding for the TPF1-mutated NS4B polypeptide having the above-described adaptive mutations as the replicon RNA, the replication efficiency of the RNA increases.

### Example 5

### Construction of HCV Particle Capable of Reinfection

Whether or not the core antigen secreted into the culture medium in Example 4 was capable of forming the virus particle and of in vitro reinfection was examined. Specifically, the full-length HCV RNA synthesized from pTPF1/4B was transfected into Huh7 cells. After culturing the cells for 72 hours, culture supernatant was collected. The collected culture supernatant was centrifuged at 2 k rpm for 10 minutes and then filtered (0.45 pill, Millipore) to remove broken cells and the like.

The filtered supernatant was allowed to react with Huh7 cells cultured in a 12-well plate (diameter 22.1 μm) for three hours at 4° C. After the reaction, the plate was transferred to an incubator with 5% CO₂ conditions at 37° C. to culture the cells. Four hours, 24 hours, 48 hours, 72 hours and 96 hours later, the cells were detached by 1 mM EDTA-PBS and collected by centrifugation. Cell pellet was dissolved in 50 μl of RIPA buffer (20 mM Tris-HCl (pH 7.5), 150 mM NaCl, 1 mM EDTA, 1% NP40, 0.1% Deoxycholate, 0.1% SDS, Complete protease inhibitor cocktail (Roche diagnostics corporation) and supernatant was collected by centrifugation at 10 k rpm for 5 minutes. The supernatant (5 μl) was measured using a kit for the HCV core antigen (FUJIREBIO, Lumipulse).

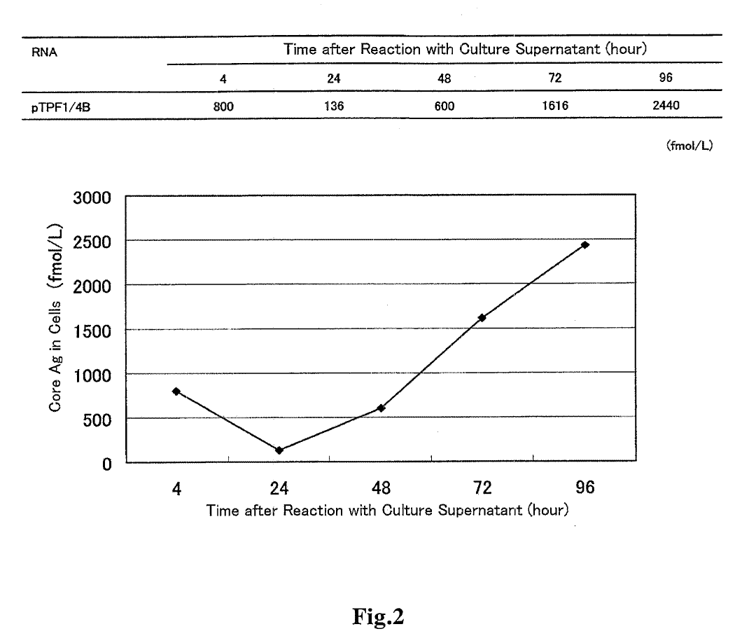

As shown in FIG. 2, the core antigen in the cells treated with the culture supernatant of pTPF1/4B exhibited a decrease once from the 4 hour to the 24 hour after the start of the culturing. Thereafter, it began to increase at the 48 hour and remained increasing at the 96 hour. This indicates that in the culture supernatant into which the full-length HCV RNA according to the present invention replicated in the cells and was secreted, the virus particles capable of reinfecting naive Huh7 cells are contained.

### Example 6

### Cloning of HCV Full-Length Genome from Patients with Interferon-Sensitive Acute Hepatitis

Cloning of the HCV full-length genome from patients with acute hepatitis for whom an interferon treatment was effective was attempted. RNA was extracted from patient's serum using an RNA extracting reagent, ISOGEN-LS (Nippon Gene), in accordance with the appended instructions.

The HCV gene from the 85th to the 9302nd nucleotide was obtained from this RNA in the same manner as described in Example 1 in which the full-length genome was obtained from TPF1. The obtained RNA was cloned into the pGEM-T easy vector. When the nucleic acid sequence was determined, it was found to be a typical full-length genome belonging to the genotype 1b.

Subsequently, the nucleic acid sequence of the 3′ untranslated region was determined. To the extracted RNA (2.5 μl), was added 5 pmole (0.5 μl) of primer 8913F, and the mixture was kept at 70° C. for 3 minutes and quickly cooled on ice. To the resultant, was added 5× First-Strand Buffer 2 μl, 0.1 M DTT 1 μl, 20 mM dNTP 0.5 μl, RNase Inhibitor (Takara Shuzo) 20 units and SuperSucript II reverse transcriptase (Invitrogen) 0.5 μl, and then sterilized water treated with diethylpyrocarbonate was added to the mixture to attain a final volume of 10 μl. This mixture was allowed to react at 42° C. for 60 minutes. To digest RNA, 12 U of RNaseH (Takara Shuzo, 60 U/μl) was added to the mixture. The mixture was kept at 37° C. for 30 minutes, followed by incubation at 72° C. for three minutes to inactivate the RNaseH. The resultant was used as cDNA.

This cDNA (2 μL) was subjected to PCR using primers 8913F and RP2 in the same manner as described above. An aliquot of this PCR product was subjected to the second PCR using 8939F and R1 primers, thereby obtaining a PCR product with about 600 bases. This PCR product was cloned into the pGEM-T Easy vector to determine the sequence.

On the other hand, the 5′ terminus of the HCV cDNA was isolated and the nucleic acid sequence was determined as follows: An aliquot of the cDNA reaction mixture treated with the above-described RNaseH was subjected to PCR using HCLongH1 and HC705R with Takara EX Taq DNA polymerase (Takara Shuzo), which PCR involved 35 repeated rounds of a thermal cycle composed of 94° C. for 20 seconds, 55° C. for 30 seconds and 72° C. for 1 minute, to amplify a fragment corresponding to the nucleotides from the first to the 709th nucleotide of the HCV cDNA previously reported. This PCR product was cloned into the pGEM-T Easy vector to determine the sequence.

By the operations described above, the entire virus genome was obtained. This was named AHC1 strain. The AHC1 strain had a full-length of 9594 nucleotide and the determined nucleic acid sequence of the entire virus genome had a translated region coding for 3010 consecutive amino acids between the 342nd and the 9374th nucleotide. The nucleic acid sequence is shown in SEQ ID NO: 10, and the amino acid sequence is shown in SEQ ID NO: 11.

The primers used for the cloning and gene analysis are shown below.

XR58R (SEQ ID NO: 12):
5′-tcatgcggct cacggacctt tcacagctag-3′
HClongA1 (SEQ ID NO: 13):
5′-atcgtcttca cgcagaaagc gtctagccat-3′
1b9405R (SEQ ID NO: 14):
5′-gcctattggc ctggagtgtt tagctc-3′
HC85F (SEQ ID NO: 15):
5′-atggcgttag tatgagtgtc gtgcagcct-3′
HC9302R (SEQ ID NO: 16):
5′-tcgggcacga gacaggctgt gatatatgtc t-3′
HC8913F (SEQ ID NO: 47):
5′-cttgaaaaag ccctggattg tcagat-3′
HC8939F (SEQ ID NO: 46):
5′-ctacggggcc tgttactcca ttgaac-3′
R1 (SEQ ID NO: 48):
5′-acatgatctg cagagaggcc agtatcagca ctctc-3′
HClongH1 (SEQ ID NO: 49):
5′-gccagccccc tgatgggggc gacactccac c-3′
HC705R (SEQ ID NO: 50):
5′-agccgcatgt aagggtatcg atgac-3′
RP2 (SEQ ID NO: 51):
5′-acatgatctg cagagaggcc-3′
M13 PrimerM3 (SEQ ID NO: 24):
5′-gtaaaacgac ggccagt-3′
M13 PrimerRV (SEQ ID NO: 25):
5′-caggaaacag ctatgac-3′
HC161S (SEQ ID NO: 52):
5′-gagtacaccggaattgccaggacgaccggg-3′
104 (SEQ ID NO: 26):
5′-aggaagactt ccgagcggtc-3′
HC841S (SEQ ID NO: 27):
5′-ggaacttgcc cggttgctct ttctctatct tc-3′
HC1405S (SEQ ID NO: 53):
5′-attccatggt ggggaactgg gccaa-3′
E1 (SEQ ID NO: 28):
5′-attccatggt ggggaactgg gctaa-3′
HC2006AS (SEQ ID NO: 54):
5′-catccatgtg cagccgaacc aatt-3′
HC2199AS (SEQ ID NO: 55):
5′-aggggtagtg ccaaagcctg tatgggtagt-3′
HC2430S (SEQ ID NO: 30):
5′-aacatcgtgg acgtgcaata cctgtacgg-3′
HC2769S (SEQ ID NO: 32):
5′-ttggaccggg agatggctgc atcgtg-3′
HC3111AS (SEQ ID NO: 56):
5′-ataatgaccc ccggcgactt tccgcactaa c-3′
HC3591AS (SEQ ID NO: 57):
5′-catggtagac agtccagcac-3′
HC4016AS (SEQ ID NO: 35):
5′-cacttggaat gtctgcggta-3′
HC4498S (SEQ ID NO: 36):
5′-agggggggag gcatctcatt ttctg-3′
HC4888F (SEQ ID NO: 37):
5′-tgctatgacg cgggctgtgc ttggta-3′
1b5290AS (SEQ ID NO: 58):
5′-gacatgcatg tcatgatgta tttg-3′
HC5950AS (SEQ ID NO: 59):
5′-ctcatgacct taaaggccac-3′
HC5858F (SEQ ID NO: 40):
5′-tggcagcata ggccttggga aggt-3′
HC6315F (SEQ ID NO: 41):
5′-aagacctggc tccagtccaa g-3′
5A-1 (SEQ ID NO: 42):
5′-ttccatgctc accgacccct c-3′
HC7090AS (SEQ ID NO: 43):
5′-accttattct ctgactccac-3′
HC7743F (SEQ ID NO: 44):
5′-cagaagaagg tcacctttgac-3′
HC8192S (SEQ ID NO: 45):
5′-gcagcgggtc gagttcctgg tgaat-3′

### Example 7

### Inhibition of Replication of HCV RNA Replicon by Interferon

Evaluation of an inhibitory action of interferon on the replication of the HCV RNA replicon was attempted using the full-length HCV RNA replicon. The full-length HCV RNA used for the evaluation of the inhibitory action of interferon was pTPF1/4B which was replicable with high efficiency in Huh7 cells in Example 4 and a chimera between AHC1 obtained in Example 6 and pTPF1/4B was also used. As for a chimera vector, the full-length HCV DNA pTPF1 was digested with restriction enzymes, Age I and Bsr GI and a fragment obtained by digesting AHC1 with restriction enzymes, Age I and Bsr GI were ligated to and inserted into the region of the cleavage of pTPF1, thereby preparing HCV DNA pTPF1/AHC1_AgeBsr in which the structural protein region of AHC1 was inserted.

The full-length HCV RNAs of pTPF1/4B and pTPF1/AHC1 AgeBsr were synthesized using the same method as in Example 2 and transfected into Huh7 cells. The transfected cells were immediately resuspended in 15 ml of the culture medium and plated 1 ml each in a 12-well plate (diameter 22.1 mm) to start a culture.

At twenty four hours after the culture was started, the medium was replaced with culture medium dissolving various concentrations (from 0.1 IU/ml to 300 IU/ml) of interferon. After culturing for another 24 hours, the cells were detached by 1 mM EDTA-PBS and collected by centrifugation. Cell pellet was dissolved in 50 μl of RIPA buffer (20 mM Tris-HCl (pH 7.5), 150 mM NaCl, 1 mM EDTA, 1% NP40, 0.1% Deoxycholate, 0.1% SDS, Complete protease inhibitor cocktail (Roche diagnostics corporation) and supernatant was collected by centrifugation at 10 k rpm for 5 minutes. The supernatant in an amount of 5 μl was measured using a kit for HCV core antigen (FUJIREBIO, Lumipulse).

The concentration of interferon at which the amount of the core antigen in the cells exhibits 50% of the amount of the core antigen in a control (a group with no interferon being added) was calculated based on a plot, which concentration was set to as IC50. The results are shown in Table 2.

TABLE 2

| | Inhibitory activity for replication of |
|---|---|
| RNA | HCV RNA replicon: IC50 (IU/m) |
| pTPF1/4B | >300 |
| pTPF1/AHC1_AgeBsr | 33 |

The results of the above-described experiment revealed that pTPF1/4B was resistant to interferon whereas pTPF 1/AHC1_AgeBsr was sensitive to interferon. From this, it is thought that the full-length HCV RNA replicon according to the present invention is an interferon-resistant RNA replicon and useful in development of a therapeutic agent against a HCV showing the interferon resistance.

### Example 8

### Inhibition of Replication of HCV RNA Replicon by Cyclosporin A

Evaluation of inhibitory action by cyclosporin A on the replication of the HCV RNA replicon was attempted. pTPF1/4B was used for the evaluation. Huh7 cells were transfected using the same method as described in Example 2 and plated in a 12-well plate. After culturing for 4 hours, the medium was replaced with a medium dissolving 1 μg of cyclosporin A.

For the cells at culturing for 24 hours, 48 hours, and 72 hours after the replacement of the culture medium containing cyclosporin A, the amount of the core antigen in cells was measured using the same method as described in Example 7, using a kit for HCV core antigen (FUJIREBIO, Lumipulse).

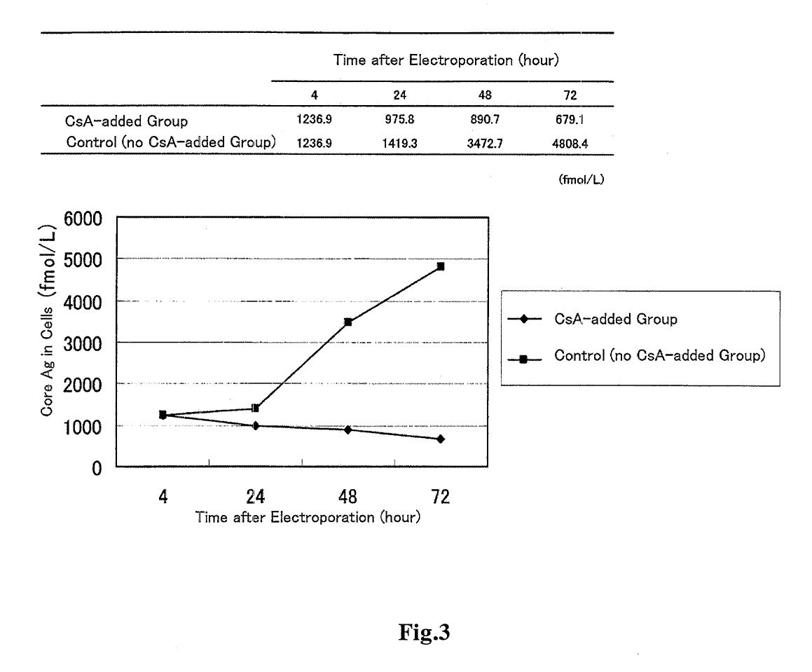

As shown in FIG. 3, it was confirmed that the group in which cyclosporin A (CsA) was added reached a maximum at 4 hours after transfection and thereafter decreased. On the other hand, it was confirmed that a control group (a group in which CsA was not added) once decreased from 4 hours to 24 hours and started increasing from 48 hours and remained increasing 72 hours later. Therefore, it was confirmed that CsA had anti-HCV activity. This indicated that the full-length HCV RNA according to the present invention can be a test system (screening method) for a therapeutic agent for HCV, which agent is thus far reported. In addition, it can be used as a test system for screening various agents affecting on the replication of HCV and/or the translation of a HCV protein.

### Example 9

### Effects of Adaptive Mutations on NS4B Protein of TPF1 Strain

Effects of the adaptive mutations of two amino acids in the NS4B protein which was obtained by the RNA replicon using TPF1 strain on replication of the replicon RNA in the other HCV genes of genotype 1b were evaluated. The mutations were introduced in the nucleotides such that the corresponding mutations were introduced in a protein of the NS4B region with 3010 amino acids of the AHC1 strain obtained in Example 6. Specifically, using Quick Mutagenesis Kit (Stratagene) in accordance with the method recommended by the manufacturer, nucleotide mutations were introduced so as to mutate the 1804th amino acid from Q (glutamine) to L (leucine) and the 1966th amino acid from E (glutamic acid) to K (lysine). The nucleic acid sequence of RNA of the obtained clone is shown in SEQ ID NO: 61 and its amino acid sequence is shown in SEQ ID NO: 62.

The same procedures as described in Example 2 were repeated except that a full-length polynucleotide of the AHC1 strain with the obtained adaptive mutations was used, thereby obtaining a plasmid DNA pRepAHC1/4B having the adaptive mutations. RepAHC1/4B replicon RNA was obtained using a fragment obtaining by digesting this pRepAHC1/4B with a restriction enzyme Xba I as a template, and transfected into human hepatocarcinoma cells (Huh7, JCRB0403), thereby establishing a cell line in which the RepAHC1/4B replicon RNA autonomously replicates.

When the same procedures were repeated using, as a control, the full-length polynucleotide of the AHC1 strain in which the adaptive mutations were not introduced, although a cell line in which the replicon RNA autonomously replicates was established, the efficiency was about one thousandth, compared with the RepAHC1/4B replicon. Hence, it was proved that the replicon RNA efficiently replicates autonomously by introducing the nucleotide mutations coding for the two adaptive mutations of the NS4B protein into the HCV genotype 1b gene.

Subsequently, the procedures described in Example 3 were repeated except that the obtained cell line was used and the nucleotide sequence of the replicated replicon RNA was determined. As a result, it was found that the 3685th nucleotide C was mutated to T and thus the amino acid corresponding to the 1115th amino acid number in SEQ ID NO: 11 of P (proline) was mutated to L (leucine). This mutation was introduced into the above-described pRepAHC1/4B to obtain a plasmid pRepAHC1/4Bm. RepAHC1/4B which was a replicon RNA obtained from pRepAHC1/4B, and RepAHC1/4Bm which was a replicon RNA obtained from pRepAHC1/4Bm were transfected into Huh7 and the number of colonies was calculated. The nucleic acid sequence of RepAHC1/4B is shown in SEQ ID NO: 67 and the nucleic acid sequence of RepAHC1/4Bm is shown in SEQ ID NO: 68.

When 1 μg of RepAHC1/4B RNA was transfected, about 10³ G418-resistant colonies were selected whereas about 10⁶ colonies were selected when 1 μg of RepAHC1/4Bm RNA was transfected. Therefore, it was proved that the replication efficiency of the replicon RNA increased by introducing one or more other adaptive mutations in addition to two of the above-described adaptive mutations in the NS4B protein.

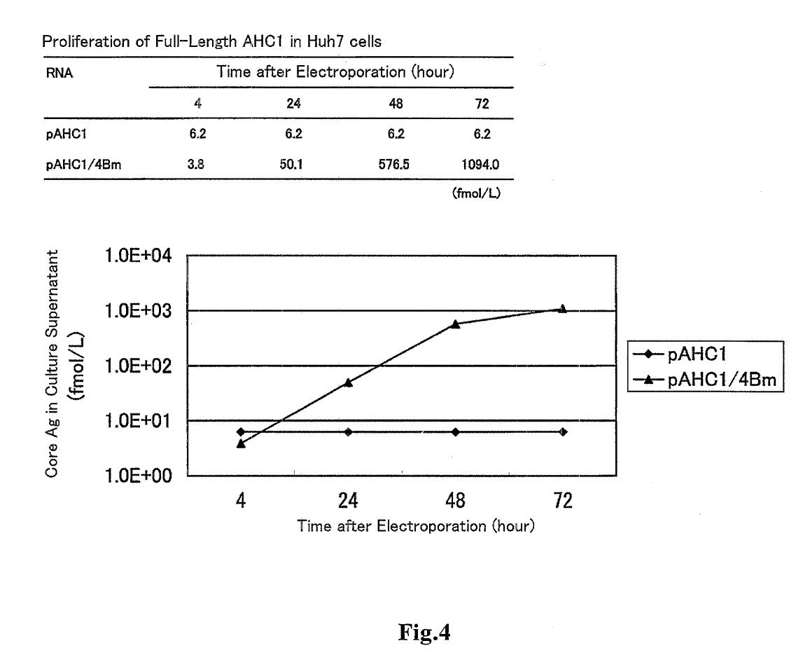

Next, the same procedures as described in Example 4 were repeated except that, as the full-length HCV DNA, pAHC1 was used instead of pTPF1 and pRepAHC1/4Bm was used instead of pRep4B, thereby preparing pAHC1 and pAHC1/4Bm which were the full-length HCV DNA. AHC1 which was a replicon RNA prepared from pAHC1 and AHC1/4Bm which was a replicon RNA prepared pAHC1/4Bm were transfected into Huh7 cells and then the amount of the core antigen in the culture supernatant was measured. The results are shown in FIG. 4.

As shown in FIG. 4, the measured value of the core antigen in the culture supernatant 24 hours, 48 hours, and 72 hours after transfection with the replicon RNA of AHC1/4Bm having the adaptive mutations was higher than the measured value of the core antigen in the culture supernatant from cells transfected with replicon RNA of AHC1 as a control. The nucleic acid sequence of AHC1/4Bm which is a replicon RNA is shown in SEQ ID NO: 63, and the encoded amino acid sequence is shown in SEQ ID NO: 64.

### INDUSTRIAL APPLICABILITY

The replicon RNA according to the present invention can be introduced into cells, can autonomously replicate and generate a HCV gene, HCV protein, and infectious particle. The replicon-replicating cells into which this replicon RNA is introduced reflect in vivo proliferation mechanisms of HCV as an in vitro model of the HCV infection. These replicon-replicating cells can be used in a method for screening a therapeutic agent of HCV. Further, the above-described method for screening can be used, besides the screening of the therapeutic agent for HCV, for quality control in the process of the production of the therapeutic agent and thus used as a method for producing a pharmaceutical.

As described above, the present invention has been illustrated along the certain modes, yet variations and modifications obvious to those skilled in the art are within the scope of the present invention.

1. A hepatitis C virus (HCV) gene comprising a polynucleotide selected from the group consisting of:
(A) a polynucleotide having the nucleic acid sequence shown in SEQ ID NO: 5;
(B) a polynucleotide having the nucleic acid sequence shown in SEQ ID NO: 7;
(C) a polynucleotide having the nucleic acid sequence shown in SEQ ID NO: 65;
(D) a polynucleotide coding for a polypeptide having the amino acid sequence shown in SEQ ID NO: 6;
(E) a polynucleotide coding for a polypeptide having the amino acid sequence shown in SEQ ID NO: 8; and
(F) a polynucleotide coding for a polypeptide having the amino acid sequence shown in SEQ ID NO: 66.

2. A HCV gene according to claim 1, said gene being a polynucleotide having the nucleic acid sequence shown in SEQ ID NO: 1, SEQ ID NO: 3, SEQ ID NO: 10, SEQ ID NO: 61 or SEQ ID NO: 63.

3. A HCV genotype 1b gene, said gene comprising nucleotides coding for the 1804th amino acid leucine and the 1966th amino acid lysine in the amino acid sequence of HCV polyprotein and a polynucleotide coding for an NS4B protein.

4. A DNA comprising a single stranded DNA having the nucleic acid sequence with uridine being substituted by thymine in the nucleic acid sequence of said HCV gene according to claim 1.

5. A polypeptide having the amino acid sequence shown in SEQ ID NO: 6, SEQ ID NO: 8 or SEQ ID NO: 66.

6. A HCV polyprotein, wherein a peptide in an NS4B region is a polypeptide having the amino acid sequence shown in SEQ ID NO: 6, SEQ ID NO: 8 or SEQ ID NO: 66.

7. At least one HCV protein selected from the group consisting of a core protein having the amino acid sequence from the first to the 191st amino acid in the amino acid sequence shown in SEQ ID NO: 2, SEQ ID NO: 4, SEQ ID NO: 11, SEQ ID NO: 62 or SEQ ID NO: 64, an E1 protein having the amino acid sequence from the 192nd to the 383rd amino acid, an E2 protein having the amino acid sequence from the 384th to the 746th amino acid, a P7 protein having the amino acid sequence from the 747th to the 809th amino acid, an NS2 protein having the amino acid sequence from the 810th to the 1026th amino acid, an NS3 protein having the amino acid sequence from the 1027th to 1657th amino acid, an NS4A protein having the amino acid sequence from the 1658th to 1711th amino acid, an NS4B protein having the amino acid sequence from the 1712th to the 1972nd amino acid, an NS5A protein having the amino acid sequence from the 1973rd to the 2419th amino acid, and an NS5B protein having the amino acid sequence from the 2420th to the 3010th amino acid.

8. A replicon RNA comprising a polynucleotide selected from the group consisting of:
(A) a polynucleotide having the nucleic acid sequence shown in SEQ ID NO: 5;
(B) a polynucleotide having the nucleic acid sequence shown in SEQ ID NO: 7;
(C) a polynucleotide having the nucleic acid sequence shown in SEQ ID NO: 65;
(D) a polynucleotide coding for a polypeptide having the amino acid sequence shown in SEQ ID NO: 6;
(E) a polynucleotide coding for a polypeptide having the amino acid sequence shown in SEQ ID NO: 8;
(F) a polynucleotide coding for a polypeptide having the amino acid sequence shown in SEQ ID NO: 66; and
(G) a polynucleotide having a nucleic acid sequence having a homology of not less than 90% with the nucleic acid sequence shown in SEQ ID NO: 5, SEQ ID NO: 7 or SEQ ID NO: 65.

9. A replicon RNA of the genotype 1b, said replicon RNA comprising nucleotides coding for the 1804th amino acid leucine and the 1966th amino acid lysine in the amino acid sequence of a HCV polyprotein and a polynucleotide coding for an NS4B protein.

10. The replicon RNA according to claim 8, said replicon RNA comprising:
(A) a polynucleotide from the first to the 341st nucleotide in the 5′ untranslated region of said HCV, a polynucleotide coding for a polypeptide from the 1027th to the 3010th amino acid in said HCV polyprotein and a polynucleotide of the 3′ untranslated region; or
(B) a polynucleotide from the first to the 341st nucleotide in the 5′ untranslated region, a polynucleotide coding for the HCV polyprotein composed of 3010 amino acids and a polynucleotide of the 3′ untranslated region.

11. The replicon RNA according to claim 8, said replicon RNA being resistant to interferon.

12. The replicon RNA according to claim 8, said replicon RNA comprising:
(A) a polynucleotide having the first to the 341st nucleotide in the nucleic acid sequence shown in SEQ ID NO: 1 and a polynucleotide having the 3420th to the 9594th nucleotide in the nucleic acid sequence shown in SEQ ID NO: 1;
(B) a polynucleotide having the first to the 341st nucleotide in the nucleic acid sequence shown in SEQ ID NO: 3 and a polynucleotide having the 3420th to the 9594th nucleotide in the nucleic acid sequence shown in SEQ ID NO: 3;
(C) a polynucleotide having the first to the 341st nucleotide in the nucleic acid sequence shown in SEQ ID NO: 10 and a polynucleotide having the 3420th to the 9594th nucleotide in the nucleic acid sequence shown in SEQ ID NO: 10;
(D) a polynucleotide having the first to the 341st nucleotide in the nucleic acid sequence shown in SEQ ID NO: 61 and a polynucleotide having the 3420th to the 9594th nucleotide in the nucleic acid sequence shown in SEQ ID NO: 61;
(E) a polynucleotide having the first to the 341st nucleotide in the nucleic acid sequence shown in SEQ ID NO: 63 and a polynucleotide having the 3420th to the 9594th nucleotide in the nucleic acid sequence shown in SEQ ID NO: 63;
(F) a polynucleotide having a nucleic acid sequence having a homology of not less than 90% with the nucleic acid sequence from the first to the 341st nucleotide in the nucleic acid sequence shown in SEQ ID NO: 1 and a polynucleotide having a nucleic acid having a homology of not less than 90% with the nucleic acid sequence from the 3420th to the 9594th nucleotide in the nucleic acid sequence shown in SEQ ID NO: 1;
(G) a polynucleotide having a nucleic acid sequence having a homology of not less than 90% with the nucleic acid sequence from the first to the 341st nucleotide in the nucleic acid sequence shown in SEQ ID NO: 3 and the polynucleotide having a nucleic acid sequence having a homology of not less than 90% with the nucleic acid sequence from the 3420th to the 9594th nucleotide in the nucleic acid sequence shown in SEQ ID NO: 3;
(H) a polynucleotide having a nucleic acid sequence having a homology of not less than 90% with the nucleic acid sequence from the first to the 341st nucleotide in the nucleic acid sequence shown in SEQ ID NO: 10 and a polynucleotide having a nucleic acid sequence having a homology of not less than 90% with the nucleic acid sequence from the 3420th to the 9594th nucleotide in the nucleic acid sequence shown in SEQ ID NO: 10;
(I) a polynucleotide having a nucleic acid sequence having a homology of not less than 90% with the nucleic acid sequence from the first to the 341st nucleotide in the nucleic acid sequence shown in SEQ ID NO: 61 and a polynucleotide having a nucleic acid sequence having a homology of not less than 90% with the nucleic acid sequence from the 3420th to the 9594th nucleotide in the nucleic acid sequence shown in SEQ ID NO: 61; or
(J) a polynucleotide having a nucleic acid sequence having a homology of not less than 90% with the nucleic acid sequence from the first to the 341st nucleotide in the nucleic acid sequence shown in SEQ ID NO: 63 and a polynucleotide having a nucleic acid sequence having a homology of not less than 90% with the nucleic acid sequence from the 3420th to the 9594th nucleotide in the nucleic acid sequence shown in SEQ ID NO: 63.

13. The replicon RNA according to claim 8, said replicon RNA being a polynucleotide having the nucleic acid sequence shown in SEQ ID NO: 1, SEQ ID NO: 3, SEQ ID NO: 10, SEQ ID NO: 61 or SEQ ID NO: 63; or a polynucleotide having a nucleic acid sequence having a homology of not less than 90% with the nucleic acid sequence shown in SEQ ID NO: 1, SEQ ID NO: 3, SEQ ID NO: 10, SEQ ID NO: 61 or SEQ ID NO: 63.

14. The replicon RNA according to claim 8, said replicon RNA comprising at least one selection marker gene or reporter gene and at least one IRES sequence.

15. A DNA coding for said replicon RNA according to claim 8.

16. A vector comprising said DNA according to claim 15.

17. A replicon-replicating cell prepared by a method comprising introducing at least one of the following into a cell:
(a) a replicon RNA according to claim 8;
(b) a DNA coding for a replicon RNA comprising a polynucleotide selected from the group consisting of: (A) a polynucleotide having the nucleic acid sequence shown in SEQ ID NO: 5; (B) a polynucleotide having the nucleic acid sequence shown in SEQ ID NO: 7; (C) a polynucleotide having the nucleic acid sequence shown in SEQ ID NO: 65; (D) a polynucleotide coding for a polypeptide having the amino acid sequence shown in SEQ ID NO: 6; (E) a polynucleotide coding for a polypeptide having the amino acid sequence in SEQ ID NO: 8; (F) a coding for a polypeptide having the amino acid sequence shown in SEQ ID NO: 66; and (G) a polynucleotide having a nucleic acid sequence having a homology of not less than 90% with the nucleic acid sequence shown in SEQ ID NO: 5, SEQ ID NO: 7 or SEQ ID NO: 65; and
(c) a vector comprising a DNA coding for a replicon RNA comprising a polynucleotide selected from the group consisting of: (A) a polynucleotide having the nucleic acid sequence shown in SEQ ID NO: 5; (B) a polynucleotide having the nucleic acid sequence shown in SEQ ID NO: 7; (C) a polynucleotide having the nucleic acid sequence shown in SEQ ID NO: 65; (D) a polynucleotide coding for a polypeptide having the amino acid sequence shown in SE ID NO: 6; (E) a polynucleotide coding for a polypeptide having the amino acid sequence shown in SEQ ID NO: 8; (F) a polynucleotide coding for a polypeptide having the amino acid sequence shown in SEQ ID NO: 66; and (G) a polynucleotide having a nucleic acid sequence having a homology of not less than 90% with the nucleic acid sequence shown in SEQ ID NO: 5, SEQ ID NO: 7 or SEQ ID NO: 65.

18. The replicon-replicating cell according to claim 17, wherein said cell is a cell derived from a hepatocyte.

19. The replicon-replicating cell according to claim 18, wherein said cell derived from said hepatocyte is an Huh-7 cell.

20. A replicon RNA produced by said replicon-replicating cell according to claim 17.

21. At least one HCV protein selected from the group consisting of CORE, E1, E2, P7, NS2, NS3, NS4A, NS4B, NS5A and NS5B produced by said replicon-replicating cell according to claim 17.

22. A HCV particle produced by said replicon-replicating cell according to claim 17.

23. A method for screening a substance controlling infection of said HCV, said method comprising the steps of contacting said replicon-replicating cell according to claim 17 with said substance and analyzing a degree of increase in said replicon RNA.

24. The method for screening according to claim 23, wherein said analysis of said degree of increase in said replicon RNA is detection of said replicon RNA or HCV protein.


SEQUENCE LISTING (68 sequences)

SEQ ID NO: 1 - RNA, 9594 nt, Hepatitis C virus
gccagcccccugaugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaucaugagcacaaauccuaaaccucaaagaaaaaccaaacguaacaccaaccgccgcccacaggacgucaaguucccgggcgguggccagaucguugguggaguuuaccuguugccgcgcaggggccccagguugggugugcgcgcgacuaggaagacuuccgagcggucgcaaccucguggaaggcgacaaccuauccccaaggcucgccagcccgagggcagggccugggcucagcccggguauccuuggccccucuauggcaacgagggucuggggugggcaggauggcuccugucaccccguggcucucggccuaguuggggccccacggacccccggcguaggucgcguaauuuggguaaggucaucgauacccucacaugcggcuucgccgaccucaugggguacauuccgcucgucggcgccccccuaggaggcgcugccagggcccuggcgcauggcguccggguucuggaggacggcgugaacuaugcaacagggaaucugcccgguugcccuuucucuaucuuccucuuagcuuugcuguccuguuugaccaucccagcuuccgcucacgaagugcgcaacguauccgggcuguaccaugucacgaacgacugcuccaacucaagcauuguguaugaggcagcggacaugaucaugcacacccccgggugcgugcccugcguccgggaggguaacuccucccgcugcuggguagcgcucacucccacgcucgcggccaggaauagcagcguccccacugcgacaauacgacgccaugucgauuugcucgucggggcggcugcuuucuguuccgcuauguacgugggggaucuuugcggaucuguuuuccucgucucccagcuguucaccuuuucaccucgccgguacgagacgguacaggacugcaauugcucacucuaucccggccacguaucaggccaucgcauggcuugggauaugaugaugaacuggucaccuacaacagccuuagugguaucgcaguuacuccggaucccacaagccgucguggauauggugguaggggcccacuggggaguccuggcgggccuugccuacuauuccaugguggggaacugggcuaaggucuugauugugaugcuacucuuugccggcgucgacgggaagaccuacgugacagggggggcgcagagccgagccacucaaggcuuugcgucccucuuuacacgggggccgucucagaaacuccagcuuguaaauuccaacggcagcuggcacauuaacaggacugccuugaacugcaaugacuccuuccagacuggguuccuugccgcgcuguuuuacgcacaccguuucaacucguccggaugcccagagcgcauggccagcugccgccccaucgacacguucgaucagggguggggccccaucacucaugucgcgcgucgcacaucggaccagaggccuuauugcuggcacuacgcaccucaaccgugugguauuguacccgcguugcagguaugugguccaguguauugcuucaccccaagccccgucgugguggggacgaccgaucgcuucggcgcccccacguacaacuggggggagaaugagacggacgugcuacuccucaacaauacgcggccgccgcacggcaacugguucggcuguacauggaugaauaguaccggguucaccaagacgugugggggcccccccugcaacaucgggggguuuggcaacaacaccuugaccugcccuacggauugcuuccggaagcaccccgaggccacuuacaccaaaugcggcucggggcccugguugacgccuaggugcaugguugauuacccauacagacuuuggcacuaccccugcacuguuaacuuuuccaucuucaaggucaggauguaugugggggguguggagcacaggcucaccgccgcgugcaauuggacucggggagagcgcugcaacuuggaggauagggacagaucggagcucagcccgcugcuacugucuaccacagaguggcagguacugcccuguucuuucaccaccuuaccggcccuguccacugguuugauccaccuccaccagaacaucguggacgugcaauaccuguacgguguggggucaucgguugucuccauugcaaucaggugggaguaugucgugcugcucuuccuccuccuggcggacgcgcgyguuugcgccugcuuguggaugaugcugcugauagcccaagcugaggccgccuuagagaaccuggugauccucaaugcggcgucuguggccggagcgcauggcguucucucuuuccuuguguucuucugcgcugccugguacaucaagggcaagcugguccccggggcggcauaugccuucuaugguguauggccgcugcuccugcuucugcugucauuaccaccacgagcauacgccuuggaccgggagauggcugcaucgugcggaggcgcgguuuucguaggucugaugcuccugaccuugucaccacacuacaagguguuucucgcuaggcucauauggugguuacaguauuuuaucaccagggccgaggcgcacuugcagguguggguccccccccucaacguucgggggggccgcgaugccaucauccuccucacgugugugguccacccagagcuaauuuuugacaucaccaaaaucuugcucgccaugcucgguccgcucauggugcuccaggcuggccuaacuagagugccguacuucguacgcgcucaagggcucauccgugcaugcauguuagugcggaaagucgcugggggccacuauguccaaauggcccucaugaaacuggccgcacugacggguacguacguuuaugaccaucuuacuccgcugcaggacugggcccacgcgggcuugcgagaccuugcaguggcaguugagcccgucgucuucucugacauggagacuaaggucaucaccuggggggcagacaccgcagcguguggggacaucaucucgggccuacccgucuccgcccgaagggggagggagauacuucugggccccgccgacagguuuggagagcagggguggcgacuccucgcgccuaucacggcuuacgcucaacagacgcggggccuacuuggcuguaucaucaccagccucacaggccgggacaagaaccaggucgagggggagguucaggugguuuccaccgcaacgcaaucuuuccuggcgaccugcgucaacggcguguguuggacugucuaccauggugccggcucgaagacccuggccggcccgaagggcccaaucacccaaauguacaccaauguggaccaagaccucgucggcuggccggcgccccccggggcgcgcucccugacaccgugcaccugcggcagcucggaccucuaccuggucacgaggcaugcugaugucauuccggugcgccggcggggcgacagcagggggagucuacucucucccaggcccaucuccuacuuaaagggcuccucaggugguccacugcuuugcccccuggggcacgcugugggcaucuuccgggccgcugugugcacccgggggguugcaaaggcgguggauuuuguaccuguugagucuauggaaaccaccaugcggucuccggucuuuacggauaauucaucucccccggccguaccgcagacauuccaaguggcccaucuacacgcucccacuggcaguggcaagagcacuaaggugccggcugcguacgcagcccaaggguacaagguacucgucuugaacccauccguugccgcuaccuuaggguuuggggcguacaugucuaaagcacaugguguugagccuaacaucagaacugggguaaggaccaucaccacgggcgcuuccaucacguauuccaccuacgguaaguuccuugccgacggugguugcucugggggcgccuaugacaucauaauaugugaugagugccacucaacugacucgacuuccaucuugggcauuggcacaguccuggaccaagcggagacggcuggagcgcggcucgucgugcucgccaccgcuacgccuccgggaucggucaccgugccacaucccaauaucgaggagguggccuugcccagcaccggagaaauucccuucuacggcaaagccauccccauugagaccaucaagggggggaggcaccucaucuucugccacuccaagaagaaaugugacgagcucgcugcaaagcuggugggccucggaguuaacgcuguugcguacuaccggggucuugauguguccgucauaccaacaagcggagaugucguugucguggcaacagacgcucuaaugacgggcuucaccggcgacuuugacucagugaucgacuguaauacuugugucacccagacaguugauuucagcuuggacccuaccuucaccauugagacgacaaccgugccccaagacgcggugucgcguucgcagcgacgaggcaggacuggcaggggcaggaugggcauauacagguuuguggcuccaggggaacggcccucgggcauguucgauucuucgguccugugugagugcuaugacgcgggcugugcuugguaugagcucacgcccgccgagaccucagucagguugcgggcuuaccuaaauacaccagggcugcccgucugccaggaccaccuggaguuuugggagggggucuucacaggccucacccacauagaugcccauuucuugucccagacuaagcaggcaggagauaacuuccccuaccugguagcauaccaggcuacggugugcgccagggcccaggcucccccuccaucgugggaucaaauguggaagugucucauacggcugaagccuacacuacacgggccaacgccccuguuguauaggcuaggagccguccagaaugaggucauccucacacaucccauaaccaaauacaucauggcaugcaugucggcugaccuagaggucgucacuagcaccugggugcuggucggcgggguccuugcagcucuggccgcguacugccugacgacgggcagcguggucauugugggcaggaucaucuuguccgggaagccggcuaucauuccugacagggaaguccucuaccgggaguucgaugaaauggaagagugugccucacaccuccccuacaucgaacagggaaugcagcucgccgaacaauucaagcagaaggcgcucggguugcugcagacagccaccaagcaagcggaagccgcugcuccugugguggaguccaaguggcgagcccuugaggccuucugggcgaagcacauguggaauuucaucagcgggauacaguacuuagcaggcuuguccacucugccugggaaccccgcgauagcaucacugauggcauucacagccucuaucaccagcccgcuuaccacccaacacacccuccuguuuaacaucuugggaggauggguggccgcccaacuugccccccccggugcugccucggcuuucgugggcgccggcauugcuggcgcagcuguuggcagcauaggccuugggaaggugcuuguggacauccuggcggguuauggagcagggguggcaggcgcgcucguggccuucaaggucaugagcggcgagaugcccuccaccgaggaccuggucaacuuacucccugccauccucucuccuggugcccuugucgucggggucgugugcgcagcaauacugcgucggcaugugggcccgggggagggggcugugcaauggaugaaccggcugauagcguucgccucgcgggguaaccacgucucccccacgcacuaugugccugagagcgacgcugcagcgcgugucacacagauccucucuagccucaccaucacucagcuacugaagaggcuccaccaguggauuaaugaggacugcuccacaccaugcuccggcucguggcuuagggacguuugggacuggauaugcacgguuuugagugacuucaagaccuggcuccaguccaagcuccugccacgguuaccgggaguuccauuccuuucaugccaacguggguauaagggggucuggcggggagauggcaucaugcagaccuccugcccauguggagcacaaaucgccggacaugucaagaacgguuccaugaggaucguugggccuaaaaccuguagcaacacguggcacggaacauuccccauuaacgcgcacaccacgggccccugcacacccuccccagcgccgaacuacucuaaggcguuguggcggguggcugcugaggaguacguggaagucacgcgggugggggauuuccauuacgugacgggcaugaccacugacaacguaaaaugcccaugccagguuccggcccccgaguucuucacagagguggaugggguacggcugcacagguacgcuccggcgugcaaaccucuccuacgggaugaggucacauuccaggucgggcucaaccaguucccgguugggucacagcucccaugcgagcccgaaccggauguaucagugcucacuuccaugcuuaccgacccuucccacaucacagcagagacggcuaagcguaggcuggccagagggucuucccccucuuuggccagcucuucagcuagucaguugucugcgcccucauugaaggcgacaugcaccacccaucaugacuccccagacgcugaccucauugaggccaaccuccuguggcggcaggagaugggagggaacaucacccguguggagucagagaacaaggugguaauccuggacucuuuugacccgcuucgagcggaggaggacgagagggaggugucuguugcggcggagauccugcggaaaaccaggaaguuccccccagcgaugcccauaugggcacgcccggacuacaacccaccgcugcuagagacuuggaaggacccggacuacgucccuccaguggugcacgggugcccauugccaccuaccaagaccccuccaauaccaccuccgcggaggaaaaagacaguuguccugacagaguccaccgugucuucugcccuggcggagcuugccacaaagaccuuuggcagcuccggaucgucggccgucgacagcggcacagcgaccgcccccccuaaccagcucuccgacgaaguggauacaggauccgacguugagucguacuccuccaugcccccccuugagggagagccgggggaccccgaucucagcgacgggucuuggucuacuguaagugaggaggcuggugaggacgucgucugcugcucgauguccuacacauggacaggcgccuugaucacgccgugcgccgcggaggagagcaagcugcccaucaaugcgcugagcaacucuuugcugcgccaccacaacauggucuaugccacaacaucccgcagcgcaagccaacggcagaaaaaggucaccuuugacagacugcaaguccuggacgaccauuaccgggacgugcucaaggagaugaaggcgaaggcguccacaguuaaggcuaaacuucuauccguagaagaggccugcaagcugacgcccccacacucagccagguccaaauuuggcuauggggcgaaggacguccggaaccuauccagcaaggccguuaaccacaucaacuccguguggaaggacuugcuggaagacacugagacaccaauugacaccaccaucauggcaaaaaaugaggucuucuguguucaaccagagaagggaggccgcaagccagcucgccuuaucguauacccagacuugggggugcgugugugcgagaaaauggcccuuuacgacguggucuccacucuuccucaggccgugaugggcuccucauacggauuccaguacucuccugggcagcgggucgaguuccuggugaaugccuggaaaucaaagaagaacccuaugggcuucgcauaugacacccgcuguuuugacucaacggucaccgagaacgacauccguguugaggagucaauuuaccaauguugugacuuggcccccgaggccagacaggugauaaggucgcucacagagcggcuuuaugucgggggcccccugacuaauucaaaagggcagaacugcgguuaucgccggugccgcgccagcggcgugcugacgacuagcugcgguaauacccucacauguuacuugaaggccucugcagccugucgagcugcaaagcuccaggacugcacgaugcucgugugcggggacgaccuugucguuaucugugaaagcgcggggacccaggaggacgcggcgagccuacgagucuucacggaggcuaugacuagguacuccgccccccccggggacccgccccgaccggaauacgacuuggaguugauaacaucaugcuccuccaacgugucggucgcgcacgaugcaucuggcaaacggguguauuaccucacccgugaccccaccaccccccuugcgcgggcugcgugggagacagcuaaacacacuccagucaacuccuggcuaggcaacaucaucauguaugcgcccacccucugggcaaggaugauucugaugacucacuucuucuccauccuucuagcucaggagcagcuugaaaaagcccuggauugucagaucuacggggccacuuacuccauugaaccacuugaccuaccucagaucauucaacgacuccauggucuuagcgcauucucacuccauaguuacucuccaggugaaaucaauaggguggcuucaugccucaggaaacuugggguaccgcccuugcgagucuggagacaucgggccagaaguguccgcgcuaagcuacugucccagggggggagggcugccacuuguggcaaguaccucuucaacugggcaguaaggaccaagcucaaacucacuccaaucccggcugcgucccaguuggacuuguccggcugguucauugcugguuacagcgggggagacauauaucacagccugucucgugcccgaccccgcugguuuauguugugccuacuccuacuuucuguggggguaggcaucuaccugcuccccaaucgaugaacggggggcuaaacacuccaggccaauaggccauucuguuuuuuuuuuuuuuuuuuuuuuuuuuuuuuuuuuuuuuuuuuuuuuuuuuuuuuccuuuuuuuuuuuuuuuuucccuuucuuuugguggcuccaucuuagcccuagucacggcuagcugugaaagguccgugagccgcaugacugcagagagugcugauacuggccucucugcagaucaugu

SEQ ID NO: 2 - protein, 3010 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCPFSIFLLALLSCLTIPASAHEVRNVSGLYHVTNDCSNSSIVYEAADMIMHTPGCVPCVREGNSSRCWVALTPTLAARNSSVPTATIRRHVDLLVGAAAFCSAMYVGDLCGSVFLVSQLFTFSPRRYETVQDCNCSLYPGHVSGHRMAWDMMMNWSPTTALVVSQLLRIPQAVVDMVVGAHWGVLAGLAYYSMVGNWAKVLIVMLLFAGVDGKTYVTGGAQSRATQGFASLFTRGPSQKLQLVNSNGSWHINRTALNCNDSFQTGFLAALFYAHRFNSSGCPERMASCRPIDTFDQGWGPITHVARRTSDQRPYCWHYAPQPCGIVPALQVCGPVYCFTPSPVVVGTTDRFGAPTYNWGENETDVLLLNNTRPPHGNWFGCTWMNSTGFTKTCGGPPCNIGGFGNNTLTCPTDCFRKHPEATYTKCGSGPWLTPRCMVDYPYRLWHYPCTVNFSIFKVRMYVGGVEHRLTAACNWTRGERCNLEDRDRSELSPLLLSTTEWQVLPCSFTTLPALSTGLIHLHQNIVDVQYLYGVGSSVVSIAIRWEYVVLLFLLLADARVCACLWMMLLIAQAEAALENLVILNAASVAGAHGVLSFLVFFCAAWYIKGKLVPGAAYAFYGVWPLLLLLLSLPPRAYALDREMAASCGGAVFVGLMLLTLSPHYKVFLARLIWWLQYFITRAEAHLQVWVPPLNVRGGRDAIILLTCVVHPELIFDITKILLAMLGPLMVLQAGLTRVPYFVRAQGLIRACMLVRKVAGGHYVQMALMKLAALTGTYVYDHLTPLQDWAHAGLRDLAVAVEPVVFSDMETKVITWGADTAACGDIISGLPVSARRGREILLGPADRFGEQGWRLLAPITAYAQQTRGLLGCIITSLTGRDKNQVEGEVQVVSTATQSFLATCVNGVCWTVYHGAGSKTLAGPKGPITQMYTNVDQDLVGWPAPPGARSLTPCTCGSSDLYLVTRHADVIPVRRRGDSRGSLLSPRPISYLKGSSGGPLLCPLGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVEPNIRTGVRTITTGASITYSTYGKFLADGGCSGGAYDIIICDECHSTDSTSILGIGTVLDQAETAGARLVVLATATPPGSVTVPHPNIEEVALPSTGEIPFYGKAIPIETIKGGRHLIFCHSKKKCDELAAKLVGLGVNAVAYYRGLDVSVIPTSGDVVVVATDALMTGFTGDFDSVIDCNTCVTQTVDFSLDPTFTIETTTVPQDAVSRSQRRGRTGRGRMGIYRFVAPGERPSGMFDSSVLCECYDAGCAWYELTPAETSVRLRAYLNTPGLPVCQDHLEFWEGVFTGLTHIDAHFLSQTKQAGDNFPYLVAYQATVCARAQAPPPSWDQMWKCLIRLKPTLHGPTPLLYRLGAVQNEVILTHPITKYIMACMSADLEVVTSTWVLVGGVLAALAAYCLTTGSVVIVGRIILSGKPAIIPDREVLYREFDEMEECASHLPYIEQGMQLAEQFKQKALGLLQTATKQAEAAAPVVESKWRALEAFWAKHMWNFISGIQYLAGLSTLPGNPAIASLMAFTASITSPLTTQHTLLFNILGGWVAAQLAPPGAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLSDFKTWLQSKLLPRLPGVPFLSCQRGYKGVWRGDGIMQTSCPCGAQIAGHVKNGSMRIVGPKTCSNTWHGTFPINAHTTGPCTPSPAPNYSKALWRVAAEEYVEVTRVGDFHYVTGMTTDNVKCPCQVPAPEFFTEVDGVRLHRYAPACKPLLRDEVTFQVGLNQFPVGSQLPCEPEPDVSVLTSMLTDPSHITAETAKRRLARGSSPSLASSSASQLSAPSLKATCTTHHDSPDADLIEANLLWRQEMGGNITRVESENKVVILDSFDPLRAEEDEREVSVAAEILRKTRKFPPAMPIWARPDYNPPLLETWKDPDYVPPVVHGCPLPPTKTPPIPPPRRKKTVVLTESTVSSALAELATKTFGSSGSSAVDSGTATAPPNQLSDEVDTGSDVESYSSMPPLEGEPGDPDLSDGSWSTVSEEAGEDVVCCSMSYTWTGALITPCAAEESKLPINALSNSLLRHHNMVYATTSRSASQRQKKVTFDRLQVLDDHYRDVLKEMKAKASTVKAKLLSVEEACKLTPPHSARSKFGYGAKDVRNLSSKAVNHINSVWKDLLEDTETPIDTTIMAKNEVFCVQPEKGGRKPARLIVYPDLGVRVCEKMALYDVVSTLPQAVMGSSYGFQYSPGQRVEFLVNAWKSKKNPMGFAYDTRCFDSTVTENDIRVEESIYQCCDLAPEARQVIRSLTERLYVGGPLTNSKGQNCGYRRCRASGVLTTSCGNTLTCYLKASAACRAAKLQDCTMLVCGDDLVVICESAGTQEDAASLRVFTEAMTRYSAPPGDPPRPEYDLELITSCSSNVSVAHDASGKRVYYLTRDPTTPLARAAWETAKHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAATCGKYLFNWAVRTKLKLTPIPAASQLDLSGWFIAGYSGGDIYHSLSRARPRWFMLCLLLLSVGVGIYLLPNR

SEQ ID NO: 3 - RNA, 9594 nt, Hepatitis C virus
gccagcccccugaugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaucaugagcacaaauccuaaaccucaaagaaaaaccaaacguaacaccaaccgccgcccacaggacgucaaguucccgggcgguggccagaucguugguggaguuuaccuguugccgcgcaggggccccagguugggugugcgcgcgacuaggaagacuuccgagcggucgcaaccucguggaaggcgacaaccuauccccaaggcucgccagcccgagggcagggccugggcucagcccggguauccuuggccccucuauggcaacgagggucuggggugggcaggauggcuccugucaccccguggcucucggccuaguuggggccccacggacccccggcguaggucgcguaauuuggguaaggucaucgauacccucacaugcggcuucgccgaccucaugggguacauuccgcucgucggcgccccccuaggaggcgcugccagggcccuggcgcauggcguccggguucuggaggacggcgugaacuaugcaacagggaaucugcccgguugcccuuucucuaucuuccucuuagcuuugcuguccuguuugaccaucccagcuuccgcucacgaagugcgcaacguauccgggcuguaccaugucacgaacgacugcuccaacucaagcauuguguaugaggcagcggacaugaucaugcacacccccgggugcgugcccugcguccgggaggguaacuccucccgcugcuggguagcgcucacucccacgcucgcggccaggaauagcagcguccccacugcgacaauacgacgccaugucgauuugcucgucggggcggcugcuuucuguuccgcuauguacgugggggaucuuugcggaucuguuuuccucgucucccagcuguucaccuuuucaccucgccgguacgagacgguacaggacugcaauugcucacucuaucccggccacguaucaggccaucgcauggcuugggauaugaugaugaacuggucaccuacaacagccuuagugguaucgcaguuacuccggaucccacaagccgucguggauauggugguaggggcccacuggggaguccuggcgggccuugccuacuauuccaugguggggaacugggcuaaggucuugauugugaugcuacucuuugccggcgucgacgggaagaccuacgugacagggggggcgcagagccgagccacucaaggcuuugcgucccucuuuacacgggggccgucucagaaacuccagcuuguaaauuccaacggcagcuggcacauuaacaggacugccuugaacugcaaugacuccuuccagacuggguuccuugccgcgcuguuuuacgcacaccguuucaacucguccggaugcccagagcgcauggccagcugccgccccaucgacacguucgaucagggguggggccccaucacucaugucgcgcgucgcacaucggaccagaggccuuauugcuggcacuacgcaccucaaccgugugguauuguacccgcguugcagguaugugguccaguguauugcuucaccccaagccccgucgugguggggacgaccgaucgcuucggcgcccccacguacaacuggggggagaaugagacggacgugcuacuccucaacaauacgcggccgccgcacggcaacugguucggcuguacauggaugaauaguaccggguucaccaagacgugugggggcccccccugcaacaucgggggguuuggcaacaacaccuugaccugcccuacggauugcuuccggaagcaccccgaggccacuuacaccaaaugcggcucggggcccugguugacgccuaggugcaugguugauuacccauacagacuuuggcacuaccccugcacuguuaacuuuuccaucuucaaggucaggauguaugugggggguguggagcacaggcucaccgccgcgugcaauuggacucggggagagcgcugcaacuuggaggauagggacagaucggagcucagcccgcugcuacugucuaccacagaguggcagguacugcccuguucuuucaccaccuuaccggcccuguccacugguuugauccaccuccaccagaacaucguggacgugcaauaccuguacgguguggggucaucgguugucuccauugcaaucaggugggaguaugucgugcugcucuuccuccuccuggcggacgcgcgyguuugcgccugcuuguggaugaugcugcugauagcccaagcugaggccgccuuagagaaccuggugauccucaaugcggcgucuguggccggagcgcauggcguucucucuuuccuuguguucuucugcgcugccugguacaucaagggcaagcugguccccggggcggcauaugccuucuaugguguauggccgcugcuccugcuucugcugucauuaccaccacgagcauacgccuuggaccgggagauggcugcaucgugcggaggcgcgguuuucguaggucugaugcuccugaccuugucaccacacuacaagguguuucucgcuaggcucauauggugguuacaguauuuuaucaccagggccgaggcgcacuugcagguguggguccccccccucaacguucgggggggccgcgaugccaucauccuccucacgugugugguccacccagagcuaauuuuugacaucaccaaaaucuugcucgccaugcucgguccgcucauggugcuccaggcuggccuaacuagagugccguacuucguacgcgcucaagggcucauccgugcaugcauguuagugcggaaagucgcugggggccacuauguccaaauggcccucaugaaacuggccgcacugacggguacguacguuuaugaccaucuuacuccgcugcaggacugggcccacgcgggcuugcgagaccuugcaguggcaguugagcccgucgucuucucugacauggagacuaaggucaucaccuggggggcagacaccgcagcguguggggacaucaucucgggccuacccgucuccgcccgaagggggagggagauacuucugggccccgccgacagguuuggagagcagggguggcgacuccucgcgccuaucacggcuuacgcucaacagacgcggggccuacuuggcuguaucaucaccagccucacaggccgggacaagaaccaggucgagggggagguucaggugguuuccaccgcaacgcaaucuuuccuggcgaccugcgucaacggcguguguuggacugucuaccauggugccggcucgaagacccuggccggcccgaagggcccaaucacccaaauguacaccaauguggaccaagaccucgucggcuggccggcgccccccggggcgcgcucccugacaccgugcaccugcggcagcucggaccucuaccuggucacgaggcaugcugaugucauuccggugcgccggcggggcgacagcagggggagucuacucucucccaggcccaucuccuacuuaaagggcuccucaggugguccacugcuuugcccccuggggcacgcugugggcaucuuccgggccgcugugugcacccgggggguugcaaaggcgguggauuuuguaccuguugagucuauggaaaccaccaugcggucuccggucuuuacggauaauucaucucccccggccguaccgcagacauuccaaguggcccaucuacacgcucccacuggcaguggcaagagcacuaaggugccggcugcguacgcagcccaaggguacaagguacucgucuugaacccauccguugccgcuaccuuaggguuuggggcguacaugucuaaagcacaugguguugagccuaacaucagaacugggguaaggaccaucaccacgggcgcuuccaucacguauuccaccuacgguaaguuccuugccgacggugguugcucugggggcgccuaugacaucauaauaugugaugagugccacucaacugacucgacuuccaucuugggcauuggcacaguccuggaccaagcggagacggcuggagcgcggcucgucgugcucgccaccgcuacgccuccgggaucggucaccgugccacaucccaauaucgaggagguggccuugcccagcaccggagaaauucccuucuacggcaaagccauccccauugagaccaucaagggggggaggcaccucaucuucugccacuccaagaagaaaugugacgagcucgcugcaaagcuggugggccucggaguuaacgcuguugcguacuaccggggucuugauguguccgucauaccaacaagcggagaugucguugucguggcaacagacgcucuaaugacgggcuucaccggcgacuuugacucagugaucgacuguaauacuugugucacccagacaguugauuucagcuuggacccuaccuucaccauugagacgacaaccgugccccaagacgcggugucgcguucgcagcgacgaggcaggacuggcaggggcaggaugggcauauacagguuuguggcuccaggggaacggcccucgggcauguucgauucuucgguccugugugagugcuaugacgcgggcugugcuugguaugagcucacgcccgccgagaccucagucagguugcgggcuuaccuaaauacaccagggcugcccgucugccaggaccaccuggaguuuugggagggggucuucacaggccucacccacauagaugcccauuucuugucccagacuaagcaggcaggagauaacuuccccuaccugguagcauaccaggcuacggugugcgccagggcccaggcucccccuccaucgugggaucaaauguggaagugucucauacggcugaagccuacacuacacgggccaacgccccuguuguauaggcuaggagccguccagaaugaggucauccucacacaucccauaaccaaauacaucauggcaugcaugucggcugaccuagaggucgucacuagcaccugggugcuggucggcgggguccuugcagcucuggccgcguacugccugacgacgggcagcguggucauugugggcaggaucaucuuguccgggaagccggcuaucauuccugacagggaaguccucuaccgggaguucgaugaaauggaagagugugccucacaccuccccuacaucgaacagggaaugcagcucgccgaacaauucaagcagaaggcgcucggguugcugcagacagccaccaagcaagcggaagccgcugcuccugugguggaguccaaguggcgagcccuugaggccuucugggcgaagcacauguggaauuucaucagcgggauacaguacuuagcaggcuuguccacucugccugggaaccccgcgauagcaucacugauggcauucacagccucuaucaccagcccgcuuaccacccuacacacccuccuguuuaacaucuugggaggauggguggccgcccaacuugccccccccggugcugccucggcuuucgugggcgccggcauugcuggcgcagcuguuggcagcauaggccuugggaaggugcuuguggacauccuggcggguuauggagcagggguggcaggcgcgcucguggccuucaaggucaugagcggcgagaugcccuccaccgaggaccuggucaacuuacucccugccauccucucuccuggugcccuugucgucggggucgugugcgcagcaauacugcgucggcaugugggcccgggggagggggcugugcaauggaugaaccggcugauagcguucgccucgcgggguaaccacgucucccccacgcacuaugugccugagagcgacgcugcagcgcgugucacacagauccucucuagccucaccaucacucagcuacugaagaggcuccaccaguggauuaauaaggacugcuccacaccaugcuccggcucguggcuuagggacguuugggacuggauaugcacgguuuugagugacuucaagaccuggcuccaguccaagcuccugccacgguuaccgggaguuccauuccuuucaugccaacguggguauaagggggucuggcggggagauggcaucaugcagaccuccugcccauguggagcacaaaucgccggacaugucaagaacgguuccaugaggaucguugggccuaaaaccuguagcaacacguggcacggaacauuccccauuaacgcgcacaccacgggccccugcacacccuccccagcgccgaacuacucuaaggcguuguggcggguggcugcugaggaguacguggaagucacgcgggugggggauuuccauuacgugacgggcaugaccacugacaacguaaaaugcccaugccagguuccggcccccgaguucuucacagagguggaugggguacggcugcacagguacgcuccggcgugcaaaccucuccuacgggaugaggucacauuccaggucgggcucaaccaguucccgguugggucacagcucccaugcgagcccgaaccggauguaucagugcucacuuccaugcuuaccgacccuucccacaucacagcagagacggcuaagcguaggcuggccagagggucuucccccucuuuggccagcucuucagcuagucaguugucugcgcccucauugaaggcgacaugcaccacccaucaugacuccccagacgcugaccucauugaggccaaccuccuguggcggcaggagaugggagggaacaucacccguguggagucagagaacaaggugguaauccuggacucuuuugacccgcuucgagcggaggaggacgagagggaggugucuguugcggcggagauccugcggaaaaccaggaaguuccccccagcgaugcccauaugggcacgcccggacuacaacccaccgcugcuagagacuuggaaggacccggacuacgucccuccaguggugcacgggugcccauugccaccuaccaagaccccuccaauaccaccuccgcggaggaaaaagacaguuguccugacagaguccaccgugucuucugcccuggcggagcuugccacaaagaccuuuggcagcuccggaucgucggccgucgacagcggcacagcgaccgcccccccuaaccagcucuccgacgaaguggauacaggauccgacguugagucguacuccuccaugcccccccuugagggagagccgggggaccccgaucucagcgacgggucuuggucuacuguaagugaggaggcuggugaggacgucgucugcugcucgauguccuacacauggacaggcgccuugaucacgccgugcgccgcggaggagagcaagcugcccaucaaugcgcugagcaacucuuugcugcgccaccacaacauggucuaugccacaacaucccgcagcgcaagccaacggcagaaaaaggucaccuuugacagacugcaaguccuggacgaccauuaccgggacgugcucaaggagaugaaggcgaaggcguccacaguuaaggcuaaacuucuauccguagaagaggccugcaagcugacgcccccacacucagccagguccaaauuuggcuauggggcgaaggacguccggaaccuauccagcaaggccguuaaccacaucaacuccguguggaaggacuugcuggaagacacugagacaccaauugacaccaccaucauggcaaaaaaugaggucuucuguguucaaccagagaagggaggccgcaagccagcucgccuuaucguauacccagacuugggggugcgugugugcgagaaaauggcccuuuacgacguggucuccacucuuccucaggccgugaugggcuccucauacggauuccaguacucuccugggcagcgggucgaguuccuggugaaugccuggaaaucaaagaagaacccuaugggcuucgcauaugacacccgcuguuuugacucaacggucaccgagaacgacauccguguugaggagucaauuuaccaauguugugacuuggcccccgaggccagacaggugauaaggucgcucacagagcggcuuuaugucgggggcccccugacuaauucaaaagggcagaacugcgguuaucgccggugccgcgccagcggcgugcugacgacuagcugcgguaauacccucacauguuacuugaaggccucugcagccugucgagcugcaaagcuccaggacugcacgaugcucgugugcggggacgaccuugucguuaucugugaaagcgcggggacccaggaggacgcggcgagccuacgagucuucacggaggcuaugacuagguacuccgccccccccggggacccgccccgaccggaauacgacuuggaguugauaacaucaugcuccuccaacgugucggucgcgcacgaugcaucuggcaaacggguguauuaccucacccgugaccccaccaccccccuugcgcgggcugcgugggagacagcuaaacacacuccagucaacuccuggcuaggcaacaucaucauguaugcgcccacccucugggcaaggaugauucugaugacucacuucuucuccauccuucuagcucaggagcagcuugaaaaagcccuggauugucagaucuacggggccacuuacuccauugaaccacuugaccuaccucagaucauucaacgacuccauggucuuagcgcauucucacuccauaguuacucuccaggugaaaucaauaggguggcuucaugccucaggaaacuugggguaccgcccuugcgagucuggagacaucgggccagaaguguccgcgcuaagcuacugucccagggggggagggcugccacuuguggcaaguaccucuucaacugggcaguaaggaccaagcucaaacucacuccaaucccggcugcgucccaguuggacuuguccggcugguucauugcugguuacagcgggggagacauauaucacagccugucucgugcccgaccccgcugguuuauguugugccuacuccuacuuucuguggggguaggcaucuaccugcuccccaaucgaugaacggggggcuaaacacuccaggccaauaggccauucuguuuuuuuuuuuuuuuuuuuuuuuuuuuuuuuuuuuuuuuuuuuuuuuuuuuuuuccuuuuuuuuuuuuuuuuucccuuucuuuugguggcuccaucuuagcccuagucacggcuagcugugaaagguccgugagccgcaugacugcagagagugcugauacuggccucucugcagaucaugu

SEQ ID NO: 4 - protein, 3010 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCPFSIFLLALLSCLTIPASAHEVRNVSGLYHVTNDCSNSSIVYEAADMIMHTPGCVPCVREGNSSRCWVALTPTLAARNSSVPTATIRRHVDLLVGAAAFCSAMYVGDLCGSVFLVSQLFTFSPRRYETVQDCNCSLYPGHVSGHRMAWDMMMNWSPTTALVVSQLLRIPQAVVDMVVGAHWGVLAGLAYYSMVGNWAKVLIVMLLFAGVDGKTYVTGGAQSRATQGFASLFTRGPSQKLQLVNSNGSWHINRTALNCNDSFQTGFLAALFYAHRFNSSGCPERMASCRPIDTFDQGWGPITHVARRTSDQRPYCWHYAPQPCGIVPALQVCGPVYCFTPSPVVVGTTDRFGAPTYNWGENETDVLLLNNTRPPHGNWFGCTWMNSTGFTKTCGGPPCNIGGFGNNTLTCPTDCFRKHPEATYTKCGSGPWLTPRCMVDYPYRLWHYPCTVNFSIFKVRMYVGGVEHRLTAACNWTRGERCNLEDRDRSELSPLLLSTTEWQVLPCSFTTLPALSTGLIHLHQNIVDVQYLYGVGSSVVSIAIRWEYVVLLFLLLADARVCACLWMMLLIAQAEAALENLVILNAASVAGAHGVLSFLVFFCAAWYIKGKLVPGAAYAFYGVWPLLLLLLSLPPRAYALDREMAASCGGAVFVGLMLLTLSPHYKVFLARLIWWLQYFITRAEAHLQVWVPPLNVRGGRDAIILLTCVVHPELIFDITKILLAMLGPLMVLQAGLTRVPYFVRAQGLIRACMLVRKVAGGHYVQMALMKLAALTGTYVYDHLTPLQDWAHAGLRDLAVAVEPVVFSDMETKVITWGADTAACGDIISGLPVSARRGREILLGPADRFGEQGWRLLAPITAYAQQTRGLLGCIITSLTGRDKNQVEGEVQVVSTATQSFLATCVNGVCWTVYHGAGSKTLAGPKGPITQMYTNVDQDLVGWPAPPGARSLTPCTCGSSDLYLVTRHADVIPVRRRGDSRGSLLSPRPISYLKGSSGGPLLCPLGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVEPNIRTGVRTITTGASITYSTYGKFLADGGCSGGAYDIIICDECHSTDSTSILGIGTVLDQAETAGARLVVLATATPPGSVTVPHPNIEEVALPSTGEIPFYGKAIPIETIKGGRHLIFCHSKKKCDELAAKLVGLGVNAVAYYRGLDVSVIPTSGDVVVVATDALMTGFTGDFDSVIDCNTCVTQTVDFSLDPTFTIETTTVPQDAVSRSQRRGRTGRGRMGIYRFVAPGERPSGMFDSSVLCECYDAGCAWYELTPAETSVRLRAYLNTPGLPVCQDHLEFWEGVFTGLTHIDAHFLSQTKQAGDNFPYLVAYQATVCARAQAPPPSWDQMWKCLIRLKPTLHGPTPLLYRLGAVQNEVILTHPITKYIMACMSADLEVVTSTWVLVGGVLAALAAYCLTTGSVVIVGRIILSGKPAIIPDREVLYREFDEMEECASHLPYIEQGMQLAEQFKQKALGLLQTATKQAEAAAPVVESKWRALEAFWAKHMWNFISGIQYLAGLSTLPGNPAIASLMAFTASITSPLTTLHTLLFNILGGWVAAQLAPPGAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINKDCSTPCSGSWLRDVWDWICTVLSDFKTWLQSKLLPRLPGVPFLSCQRGYKGVWRGDGIMQTSCPCGAQIAGHVKNGSMRIVGPKTCSNTWHGTFPINAHTTGPCTPSPAPNYSKALWRVAAEEYVEVTRVGDFHYVTGMTTDNVKCPCQVPAPEFFTEVDGVRLHRYAPACKPLLRDEVTFQVGLNQFPVGSQLPCEPEPDVSVLTSMLTDPSHITAETAKRRLARGSSPSLASSSASQLSAPSLKATCTTHHDSPDADLIEANLLWRQEMGGNITRVESENKVVILDSFDPLRAEEDEREVSVAAEILRKTRKFPPAMPIWARPDYNPPLLETWKDPDYVPPVVHGCPLPPTKTPPIPPPRRKKTVVLTESTVSSALAELATKTFGSSGSSAVDSGTATAPPNQLSDEVDTGSDVESYSSMPPLEGEPGDPDLSDGSWSTVSEEAGEDVVCCSMSYTWTGALITPCAAEESKLPINALSNSLLRHHNMVYATTSRSASQRQKKVTFDRLQVLDDHYRDVLKEMKAKASTVKAKLLSVEEACKLTPPHSARSKFGYGAKDVRNLSSKAVNHINSVWKDLLEDTETPIDTTIMAKNEVFCVQPEKGGRKPARLIVYPDLGVRVCEKMALYDVVSTLPQAVMGSSYGFQYSPGQRVEFLVNAWKSKKNPMGFAYDTRCFDSTVTENDIRVEESIYQCCDLAPEARQVIRSLTERLYVGGPLTNSKGQNCGYRRCRASGVLTTSCGNTLTCYLKASAACRAAKLQDCTMLVCGDDLVVICESAGTQEDAASLRVFTEAMTRYSAPPGDPPRPEYDLELITSCSSNVSVAHDASGKRVYYLTRDPTTPLARAAWETAKHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAATCGKYLFNWAVRTKLKLTPIPAASQLDLSGWFIAGYSGGDIYHSLSRARPRWFMLCLLLLSVGVGIYLLPNR

SEQ ID NO: 5 - RNA, 783 nt, Hepatitis C virus
gccucacaccuccccuacaucgaacagggaaugcagcucgccgaacaauucaagcagaaggcgcucggguugcugcagacagccaccaagcaagcggaagccgcugcuccugugguggaguccaaguggcgagcccuugaggccuucugggcgaagcacauguggaauuucaucagcgggauacaguacuuagcaggcuuguccacucugccugggaaccccgcgauagcaucacugauggcauucacagccucuaucaccagcccgcuuaccacccaacacacccuccuguuuaacaucuugggaggauggguggccgcccaacuugccccccccggugcugccucggcuuucgugggcgccggcauugcuggcgcagcuguuggcagcauaggccuugggaaggugcuuguggacauccuggcggguuauggagcagggguggcaggcgcgcucguggccuucaaggucaugagcggcgagaugcccuccaccgaggaccuggucaacuuacucccugccauccucucuccuggugcccuugucgucggggucgugugcgcagcaauacugcgucggcaugugggcccgggggagggggcugugcaauggaugaaccggcugauagcguucgccucgcgggguaaccacgucucccccacgcacuaugugccugagagcgacgcugcagcgcgugucacacagauccucucuagccucaccaucacucagcuacugaagaggcuccaccaguggauuaaugaggacugcuccacaccaugc

SEQ ID NO: 6 - protein, 261 aa, Hepatitis C virus
ASHLPYIEQGMQLAEQFKQKALGLLQTATKQAEAAAPVVESKWRALEAFWAKHMWNFISGIQYLAGLSTLPGNPAIASLMAFTASITSPLTTQHTLLFNILGGWVAAQLAPPGAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPC

SEQ ID NO: 7 - RNA, 783 nt, Hepatitis C virus
gccucacaccuccccuacaucgaacagggaaugcagcucgccgaacaauucaagcagaaggcgcucggguugcugcagacagccaccaagcaagcggaagccgcugcuccugugguggaguccaaguggcgagcccuugaggccuucugggcgaagcacauguggaauuucaucagcgggauacaguacuuagcaggcuuguccacucugccugggaaccccgcgauagcaucacugauggcauucacagccucuaucaccagcccgcuuaccacccuacacacccuccuguuuaacaucuugggaggauggguggccgcccaacuugccccccccggugcugccucggcuuucgugggcgccggcauugcuggcgcagcuguuggcagcauaggccuugggaaggugcuuguggacauccuggcggguuauggagcagggguggcaggcgcgcucguggccuucaaggucaugagcggcgagaugcccuccaccgaggaccuggucaacuuacucccugccauccucucuccuggugcccuugucgucggggucgugugcgcagcaauacugcgucggcaugugggcccgggggagggggcugugcaauggaugaaccggcugauagcguucgccucgcgggguaaccacgucucccccacgcacuaugugccugagagcgacgcugcagcgcgugucacacagauccucucuagccucaccaucacucagcuacugaagaggcuccaccaguggauuaauaaggacugcuccacaccaugc

SEQ ID NO: 8 - protein, 261 aa, Hepatitis C virus
ASHLPYIEQGMQLAEQFKQKALGLLQTATKQAEAAAPVVESKWRALEAFWAKHMWNFISGIQYLAGLSTLPGNPAIASLMAFTASITSPLTTLHTLLFNILGGWVAAQLAPPGAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINKDCSTPC

SEQ ID NO: 9 - RNA, 8010 nt, Artificial
  subgenomic HCV RNA replicon
uaauacgacucacuauagccagcccccgauugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaccaugagcacgaauccuaaaccucaaagaaaaaccaaacguaacaccaacgggcgcgccaugauugaacaagauggauugcacgcagguucuccggccgcuuggguggagaggcuauucggcuaugacugggcacaacagacaaucggcugcucugaugccgccguguuccggcugucagcgcaggggcgcccgguucuuuuugucaagaccgaccuguccggugcccugaaugaacugcaggacgaggcagcgcggcuaucguggcuggccacgacgggcguuccuugcgcagcugugcucgacguugucacugaagcgggaagggacuggcugcuauugggcgaagugccggggcaggaucuccugucaucucaccuugcuccugccgagaaaguauccaucauggcugaugcaaugcggcggcugcauacgcuugauccggcuaccugcccauucgaccaccaagcgaaacaucgcaucgagcgagcacguacucggauggaagccggucuugucgaucaggaugaucuggacgaagagcaucaggggcucgcgccagccgaacuguucgccaggcucaaggcgcgcaugcccgacggcgaggaucucgucgugacccauggcgaugccugcuugccgaauaucaugguggaaaauggccgcuuuucuggauucaucgacuguggccggcuggguguggcggaccgcuaucaggacauagcguuggcuacccgugauauugcugaagagcuuggcggcgaaugggcugaccgcuuccucgugcuuuacgguaucgccgcucccgauucgcagcgcaucgccuucuaucgccuucuugacgaguucuucugaguuuaaacagaccacaacgguuucccucuagcgggaucaauuccgccccucucccuccccccccccuaacguuacuggccgaagccgcuuggaauaaggccggugugcguuugucuauauguuauuuuccaccauauugccgucuuuuggcaaugugagggcccggaaaccuuggcccugucuucuugacgagcauuccuaggggucuuuccccucucgccaaaggaaugcaaggucuguuggaugucgugaaggaagcaguuccucugggaagcuucuugaagacaaacaacgucuguagcgacccuuugcaggcagcggaaccccccaccuggcgacaggugccucugcggccaaaagccacguguauaagauacaccugcaaaggcgguacaaccccagugccacguugugaguuggauaguuguggaaagagucaaauggcucuccucaagcguauucaacaaggggcugaaggaugcccagaagguaccccauuguaugggaucugaucuggggccucggugcacaugcucuacauguguuuagucgagguuaaaaaaacgucuaggccccccgaaccacggggacgugguuuuccuuugaaaaacacgauaauaccauggcgccuaucacggcuuacgcucaacagacgcggggccuacuuggcuguaucaucaccagccucacaggccgggacaagaaccaggucgagggggagguucaggugguuuccaccgcaacgcaaucuuuccuggcgaccugcgucaacggcguguguuggacugucuaccauggugccggcucgaagacccuggccggcccgaagggcccaaucacccaaauguacaccaauguggaccaagaccucgucggcuggccggcgccccccggggcgcgcucccugacaccgugcaccugcggcagcucggaccucuaccuggucacgaggcaugcugaugucauuccggugcgccggcggggcgacagcagggggagucuacucucucccaggcccaucuccuacuuaaagggcuccucaggugguccacugcuuugcccccuggggcacgcugugggcaucuuccgggccgcugugugcacccgggggguugcaaaggcgguggauuuuguaccuguugagucuauggaaaccaccaugcggucuccggucuuuacggauaauucaucucccccggccguaccgcagacauuccaaguggcccaucuacacgcucccacuggcaguggcaagagcacuaaggugccggcugcguacgcagcccaaggguacaagguacucgucuugaacccauccguugccgcuaccuuaggguuuggggcguacaugucuaaagcacaugguguugagccuaacaucagaacugggguaaggaccaucaccacgggcgcuuccaucacguauuccaccuacgguaaguuccuugccgacggugguugcucugggggcgccuaugacaucauaauaugugaugagugccacucaacugacucgacuuccaucuugggcauuggcacaguccuggaccaagcggagacggcuggagcgcggcucgucgugcucgccaccgcuacgccuccgggaucggucaccgugccacaucccaauaucgaggagguggccuugcccagcaccggagaaauucccuucuacggcaaagccauccccauugagaccaucaagggggggaggcaccucaucuucugccacuccaagaagaaaugugacgagcucgcugcaaagcuggugggccucggaguuaacgcuguugcguacuaccggggucuugauguguccgucauaccaacaagcggagaugucguugucguggcaacagacgcucuaaugacgggcuucaccggcgacuuugacucagugaucgacuguaauacuugugucacccagacaguugauuucagcuuggacccuaccuucaccauugagacgacaaccgugccccaagacgcggugucgcguucgcagcgacgaggcaggacuggcaggggcaggaugggcauauacagguuuguggcuccaggggaacggcccucgggcauguucgauucuucgguccugugugagugcuaugacgcgggcugugcuugguaugagcucacgcccgccgagaccucagucagguugcgggcuuaccuaaauacaccagggcugcccgucugccaggaccaccuggaguuuugggagggggucuucacaggccucacccacauagaugcccauuucuugucccagacuaagcaggcaggagauaacuuccccuaccugguagcauaccaggcuacggugugcgccagggcccaggcucccccuccaucgugggaucaaauguggaagugucucauacggcugaagccuacacuacacgggccaacgccccuguuguauaggcuaggagccguccagaaugaggucauccucacacaucccauaaccaaauacaucauggcaugcaugucggcugaccuagaggucgucacuagcaccugggugcuggucggcgggguccuugcagcucuggccgcguacugccugacgacgggcagcguggucauugugggcaggaucaucuuguccgggaagccggcuaucauuccugacagggaaguccucuaccgggaguucgaugaaauggaagagugugccucacaccuccccuacaucgaacagggaaugcagcucgccgaacaauucaagcagaaggcgcucggguugcugcagacagccaccaagcaagcggaagccgcugcuccugugguggaguccaaguggcgagcccuugaggccuucugggcgaagcacauguggaauuucaucagcgggauacaguacuuagcaggcuuguccacucugccugggaaccccgcgauagcaucacugauggcauucacagccucuaucaccagcccgcuuaccacccaacacacccuccuguuuaacaucuugggaggauggguggccgcccaacuugccccccccggugcugccucggcuuucgugggcgccggcauugcuggcgcagcuguuggcagcauaggccuugggaaggugcuuguggacauccuggcggguuauggagcagggguggcaggcgcgcucguggccuucaaggucaugagcggcgagaugcccuccaccgaggaccuggucaacuuacucccugccauccucucuccuggugcccuugucgucggggucgugugcgcagcaauacugcgucggcaugugggcccgggggagggggcugugcaauggaugaaccggcugauagcguucgccucgcgggguaaccacgucucccccacgcacuaugugccugagagcgacgcugcagcgcgugucacacagauccucucuagccucaccaucacucagcuacugaagaggcuccaccaguggauuaaugaggacugcuccacaccaugcuccggcucguggcuuagggacguuugggacuggauaugcacgguuuugagugacuucaagaccuggcuccaguccaagcuccugccacgguuaccgggaguuccauuccuuucaugccaacguggguauaagggggucuggcggggagauggcaucaugcagaccuccugcccauguggagcacaaaucgccggacaugucaagaacgguuccaugaggaucguugggccuaaaaccuguagcaacacguggcacggaacauuccccauuaacgcgcacaccacgggccccugcacacccuccccagcgccgaacuacucuaaggcguuguggcggguggcugcugaggaguacguggaagucacgcgggugggggauuuccauuacgugacgggcaugaccacugacaacguaaaaugcccaugccagguuccggcccccgaguucuucacagagguggaugggguacggcugcacagguacgcuccggcgugcaaaccucuccuacgggaugaggucacauuccaggucgggcucaaccaguucccgguugggucacagcucccaugcgagcccgaaccggauguaucagugcucacuuccaugcuuaccgacccuucccacaucacagcagagacggcuaagcguaggcuggccagagggucuucccccucuuuggccagcucuucagcuagucaguugucugcgcccucauugaaggcgacaugcaccacccaucaugacuccccagacgcugaccucauugaggccaaccuccuguggcggcaggagaugggagggaacaucacccguguggagucagagaacaaggugguaauccuggacucuuuugacccgcuucgagcggaggaggacgagagggaggugucuguugcggcggagauccugcggaaaaccaggaaguuccccccagcgaugcccauaugggcacgcccggacuacaacccaccgcugcuagagacuuggaaggacccggacuacgucccuccaguggugcacgggugcccauugccaccuaccaagaccccuccaauaccaccuccgcggaggaaaaagacaguuguccugacagaguccaccgugucuucugcccuggcggagcuugccacaaagaccuuuggcagcuccggaucgucggccgucgacagcggcacagcgaccgcccccccuaaccagcucuccgacgaaguggauacaggauccgacguugagucguacuccuccaugcccccccuugagggagagccgggggaccccgaucucagcgacgggucuuggucuacuguaagugaggaggcuggugaggacgucgucugcugcucgauguccuacacauggacaggcgccuugaucacgccgugcgccgcggaggagagcaagcugcccaucaaugcgcugagcaacucuuugcugcgccaccacaacauggucuaugccacaacaucccgcagcgcaagccaacggcagaaaaaggucaccuuugacagacugcaaguccuggacgaccauuaccgggacgugcucaaggagaugaaggcgaaggcguccacaguuaaggcuaaacuucuauccguagaagaggccugcaagcugacgcccccacacucagccagguccaaauuuggcuauggggcgaaggacguccggaaccuauccagcaaggccguuaaccacaucaacuccguguggaaggacuugcuggaagacacugagacaccaauugacaccaccaucauggcaaaaaaugaggucuucuguguucaaccagagaagggaggccgcaagccagcucgccuuaucguauacccagacuugggggugcgugugugcgagaaaauggcccuuuacgacguggucuccacucuuccucaggccgugaugggcuccucauacggauuccaguacucuccugggcagcgggucgaguuccuggugaaugccuggaaaucaaagaagaacccuaugggcuucgcauaugacacccgcuguuuugacucaacggucaccgagaacgacauccguguugaggagucaauuuaccaauguugugacuuggcccccgaggccagacaggugauaaggucgcucacagagcggcuuuaugucgggggcccccugacuaauucaaaagggcagaacugcgguuaucgccggugccgcgccagcggcgugcugacgacuagcugcgguaauacccucacauguuacuugaaggccucugcagccugucgagcugcaaagcuccaggacugcacgaugcucgugugcggggacgaccuugucguuaucugugaaagcgcggggacccaggaggacgcggcgagccuacgagucuucacggaggcuaugacuagguacuccgccccccccggggacccgccccgaccggaauacgacuuggaguugauaacaucaugcuccuccaacgugucggucgcgcacgaugcaucuggcaaacggguguauuaccucacccgugaccccaccaccccccuugcgcgggcugcgugggagacagcuaaacacacuccagucaacuccuggcuaggcaacaucaucauguaugcgcccacccucugggcaaggaugauucugaugacucacuucuucuccauccuucuagcucaggagcagcuugaaaaagcccuggauugucagaucuacggggccacuuacuccauugaaccacuugaccuaccucagaucauucaacgacuccauggucuuagcgcauucucacuccauaguuacucuccaggugaaaucaauaggguggcuucaugccucaggaaacuugggguaccgcccuugcgagucuggagacaucgggccagaaguguccgcgcuaagcuacugucccagggggggagggcugccacuuguggcaaguaccucuucaacugggcaguaaggaccaagcucaaacucacuccaaucccggcugcgucccaguuggacuuguccggcugguucauugcugguuacagcgggggagacauauaucacagccugucucgugcccgaccccgcugguuuauguugugccuacuccuacuuucuguggggguaggcaucuaccugcuccccaaucgaugaacggggggcuaaacacuccaggccaauaggccauucuguuuuuuuuuuuuuuuuuuuuuuuuuuuuuuuuuuuuuuuuuuuuuuuuuuuuuuccuuuuuuuuuuuuuuuuucccuuucuuuugguggcuccaucuuagcccuagucacggcuagcugugaaagguccgugagccgcaugacugcagagagugcugauacuggccucucugcagaucaugu

SEQ ID NO: 10 - RNA, 9594 nt, Hepatitis C virus
gccagcccccugaugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaucaugagcacaaauccuaaaccucaaagaaaaaccaaacguaacaccaaccgccgcccacaggacgucaaguucccgggcgguggucagaucguugguggaguuuaccuguugccgcgcaggggccccagguugggugugcgcgcgacuaggaagacuuccgagcggucgcaaccucgugaaaggcggcaaccuauccccaaggcucgccgccccgagggcagggccugggcucagcccggguacccuuggccccucuauggcaaugagggucuggggugggcaggguggcuccugucaccccgcggcucccggccuaauuggggccccacggacccccggcguaggucgcguaauuuggguaaggucaucgauacccucacaugcggcuucgccgaccucaugggguacauuccgcucgucggcgccccccuaggaggcguugccagggcccuggcgcauggcguccggguucuggaggacggcgugaacuaugcaacagggaauuugcccgguugcucuuucucuaucuuccucuuggcuuugcuguccuguuugacugucccagcuuccgcuuaugaagugcgcaacguguccgggacauaccaugucacgaacgacugcgccaacucaagcauuguguaugaggcagcggacauaaucaugcacaccccugggugcgugcccugcguccgggagggcaauuccucccgcugcuggguagcgcucacucccacgcucgcggccaggaacaggagcauccccacuacgacgauacgacgccacgucgauuugcucguuggggcggcugccuucugcuccgccauguacgugggggaucucugcgggucuguuuuccuagucucccaacuguucaccuucucaccucgccgguaugagacgguacaagaaugcaauugcucaaucuaucccggccacguaucaggucaccgcauggcuugggauaugaugaugaauuggucgcccacagcagcucuaguggugucacaguuacuccgaaucccacaagccgucguggauaugguggcaggggcccacugggggguucuggcgggucuugccuacuauuccaugguggggaacugggcuaaagucuugauugugaugcuacucuuugccggcguugacgggaccaccaccgugacagggggagcggcggccuuuggcaccaggagccuugcguccuucuuuacauuagggccgucucagaaaauccaacuuguaaacaccaacggcaguuggcacaucaauaggacugccuugaauuguaaugacucccuccagacuggguuccuugcugcgcuguucuaugcacacaagaucaacacgucuggaugcccagagcgcauggccagcugccgcccuauugacaaguucgcucagggguggggucccaucacccauggugcgccugacaccucggaccagaggcccuacugcuggcacuacgcaccucgaccgugugguaucguacccgcgucggagguguguggcccaguguauuguuucaccccaagcccuguagugguggggacgaccgaucgcuucggcguccccacguauagcuggggggagaauaagacggacgugcugcuccucaacaacacgcggccgccgcagggcaauugguucggcugcacauggaugaauggcaccggguucaccaagacgugugggggccccccgugugauaucgggggggucggcaacaacaccuugaccugccccacggauugcuuccggaagcaccccgaggccacuuauaccaaaugcggcucggggccuugguugacgccuaggugucuaguugacuacccauacaggcuuuggcacuaccccugcacugucaacuuuaccaucuucaaggucaggauguauguggggggcguggagcacaggcucaacgcugcgugcaacuggacucgaggagagcgcugugaccuagaggacagggacagaucagagcuuagcccgcugcuacugucuacaacagaguggcagauacugcccuguuccuucaccacccuaccggcucuguccacuggcuugauccaccuccaucagaacaucguggacgugcaauaccuguacgguguagggucagcaguugucuccguugugaucagaugggaguacguccugcugcucuuccuccuccuggcggacgcgcgcguuugugccugcuuauggaugaugcuguugauagcccaggcugaagccgccuuggagaaccugguaguccucaacgcggcguccguggccggagcgcauggcauccucucuuuccuuguguucuucugcgcugccugguacaucaaaggcaagcuagugccugggguggcguaugcucucuaugguguauggccgcugcuccugcuccugcuggcguugccacaacgggcguacgccauggaccgggagauggcugcaucgugcggaggcgcgguuuucgugggucugguacucuugaccuugucaccacacuauaaagcauuccuugccaaguccauauggugguuacaauauuuaaucaccagggccgaggcgcauuugcaaguguggguccccccccucaacguucgggggggccgcgaugccaucauccuccucacgugcgcgguccacccagagcuaauuuuugacaucaccaaaauuuugcucgccauauuuggcccgcucauggugcuccaggcuggucuaacuagagugccguacuuugugcgcgcucaagggcucauccgugugugcauguuggugcggaaagucgcugggggucauuauauccaaauggcucucaugagguuggccgcacugacgggcacguacguuuaugaucaucuuacuccgcugcgggacugggcccaugcgggcuugcgagaccuugcgguggcaguugagcccgucguuuucuccaacacagagaccaagauuaucaccuggggggcggacaccgcggcguguggggacaucaucuugggucuaccugucuccgcccgaagggggagagagguacuccuaggaccggccgauaacuuuggagagcaaggguggcgacuccuugcgcccaucacggccuacucccaacagacgcggggcuuacuuggcuguaucaucaccggccucacaggucgagacaagaaccaggucgaaggggagguucaggugguuuccaccgcaacacagucuuucuuggcaaccugcgucaacgguguguguuggacugucuaccauggugccggcucaaagacccuagccggcccgaaggggccaaucacccagauguacaccaacguagaccaagaccucguuggcuggcaggcgccccccggggcgcguuccaugacgccgugcaccugcggcagcucggaccuuuacuuggucacgaggcaugcugaugucauuccggugcgccggcggggugacagcagagggagccuacuuucccccaggcccgucuccuacuugaagggcucuucaggugguccacugcucugccccuuggggcacgucgugggcaucuuucgggcugccgugugcacccgggggguugcgaaggcgguggacuucauacccaucgagucuauggaaacuaccaugcggucuccggucuuuacggauaauucaucucccccggccguaccgcagacuuuucaaguggcccaucugcacgcccccacuggcagcggcaagagcaccaaggugccagcugcguaugcagcccagggguacaaggugcuugucuugaauccauccguugccgccaccuuggguuuuggggcguauauguccaaggcauacggugucgacccuaacauuagaacugggguaaggaccaucaccacaggcgcucccaucacguacuccaccuacggcaaguuccuugccgacggugguugcuccgggggcgcuuacgacaucauaauaugcgaugagugccacucaaccgacucgacuuccauuuugggcauuggcacgguccuggaucaagcggagacggcuggagcgcgacucgucgugcucgccaccgcuacgccuccgggaucggucacugugccacaccccaacaucgaggagguggccuuguccaacaccggagagauucccuucuauggcaaagccaucccccucgaggucaucaagggggggaggcaucucauuuucugucauucuaagaagaagugugaugagcucgcugcaaagcugucgggccuuggggucaacgcuguagcguacuaccggggucuugauguguccgucauaccaacaagcggggacgucguugucguggcaacagacgcucuaaugacgggcuacaccggugacuuugacucugugaucgacuguaauacaugugucacccagacagucgacuucagccuggaccccaccuucaccauugagacgacgaccgugccccaagacgcagugucgcgcucgcagcggcgagggaggacugguagggguagagggggcauauacagguuugugacuccaggagagcggcccucgggcauguucgauuccucgguccugugugaaugcuaugacgcgggcugugcuugguacgagcucacgcccgccgagaccucgguuaggcugcgggcuuaccuaaauacaccaggguugcccgucugccaggaccauuuggaguucugggaaggcgucuucacaggccucacucauauagaugcccacuucuugucucagacuaagcaggcaggagacaacuuccccuaccugguggcauaccaggccacagugugcgccagggcccaggcaccaccuccaucaugggaucaaauguggaagugucucauacggcuaaaaccuacacuacacgggccaacaccccuguuguacaggcuaggagccguccaaaacgaggucacccucacacaccccaugaccaaauacaucaugacaugcaugucggcugaccuagaggucgucaccagcacuugggugcuggugggcgggguccucgcagcuuuggccgcguacugcuugacaacgggcagcgugguuaucauaggcaggaucaucuuguccggaaggccggcuaucauucccgauagggaaguucucuaccaggaguucgaugaaauggaagagugcgccucacaccuucccuacaucgaacaaggaaugcagcucgccgagcaauucaagcagaaggcgcucggguuguugcaaacggccaccaagcaagcggaggccgcugcucccgugguggaguccaaauggcaagcccuugaggccuucugggcgaagcacauguggaacuucaucagcgggauacaguaucuagcaggcuuguccacucugccuggaaaccccgcgauagcaucgcugauggcauuuacagccucuaucacuagcccgcucaccacccaacauacccuucuauuuaacaucuuggggggauggguggccgcccaacucgccccccccagcgcugcuucagccuucguaggcgccggcaucgccggcgcggcuguuggcagcauaggucuugggaaggugcucguggacauccuagcggguuauggagcagggguggcaggcgcacucguggccuucaaggucaugagcggcgaagugcccuccacugaggaccuggucaacuuacucccugccauccucuccccuggugcccuggucgucggggucgugugcgcagcgauacugcgucggcaugugggcccaggggagggggccgugcaguggaugaaccggcugauagcguucgcuucgcgggguaaccacgucucccccacgcacuaugugccugagagcgacgccgcagcgcgugucacccagauccucuccagccuuaccaucacucagcugcuaaagaggcuccaccaguggauuaaugaggacuguuccacgccaugcuccgguucguggcucagggauguuugggacuggauaugcacgguuuugaccgacuucaaaaccuggcuccaguccaagcuccugccacgguugccgggacucccuuucuuuucaugucaacguggauauaaaggagucuggcugggagauggcguuaugcaaacuaccuguccauguggugcacaaaucagcggacaugucaaaaacggcuccaugaagaucguggggccuaaaaccugcagcaacacguggcacgggacguuccccaucaacgcauacaccacaggccccugcacacccuccccggcgccgaacuauuccaaggcguuguggcgaguggcugcugaggaguauguggaggucacgcgggugggggauuuccacuacgugacgggcaugaccacugacaacguaaaaugcccaugccaggucccggcccccgaauucuucacggaguuggauggggugcggcugcacagguacgcuccggcgugcaagccucucuuacgggaugaggucacauuccaggucgggcucaaccaguauccgguuggaucacagcuuccaugugagcccgagccggauguaacagugcucacuuccaugcucaccgaccccucccauauuacagcagagacggcuaagcguaggcuggccagaggguccccccccucuuuggccagcuccucagcuagccaguugucugcgccuuccuugaaggcgacaugcacuacccaccaugacuccccagaugcugaccucaucgaggccaaccuccuguggcggcaggagaugggcgggaacaucacccgcguggagucagagaacaagguaguaauucuggacucuuuugaaccgcuucgagcggaggaggaugauagggaaguguccguagcggcggagauccugcggagaaccaggaaauuccccgcagcgaugcccauaugggcacggccggacuacaacccaccacuccuagagucuuggaagaacccagacuacgucccuccagugguacacgggugcccauuaccaccuauuaaggccccuccgauaccaccuccacggagaaagaggacggucauccugacagaauccaccgugucuucugccuuggcggagcuugcuacaaagaccuuuggcagcuccggaucgucggccguugacagcggcacagcgacuggcccuccugaucaguccuccggugacggagauacaggauccgacgcugagucgugcuccuccaugcccccccuugagggggagccgggggaccccgaucucagcgacgggucuuggucuaccgugagcgaggaggccggugaggacgucgucugcugcucgauguccuacacauggacaggcgccuuaaucacaccgugcgccgcagaggagagcaagcugccuaucaacgcguugagcaacucuuugcugcgccaccacaacauggucuaugccacaacaucccgcagcgcgggccaacggcagaagaaggucacuuuugacagagugcagguccuggacgaccauuaccgggacgugcucaaggagaugaaggcgaaggcguccacaguuaaggcuaaacuucuaccuguagaagaagccugcaggcugacgcccccacacucggccagauccaaguuuggcuauggggcgaaggacguccggaaccuauccagcaaggccgucaaccacauccacuccguguggaaggacuugcuggaagacucugagacgccgauugacaccaccaucauggcaaaaaaugaggucuuuuguguucaaccagagaaaggaggccgcaagucagcucgucuuaucguauucccagacuuggggguucgugugugcgagaaaauggcccuuuacgacgugguuuccacucuuccucaggccgugaugggcuccucauacggguuccaguacucuccuggacagcgggucgaguuccuggugaaugccuggaaaucaaagaagaacccuaugggcuucgcauaugacacccgcuguuuugacucaacggucacugagagugacauccguguugaggagucaauuuaccaauguugugacuuggcccccgaggccagacaggucauaaggucgcucacggagcggcuuuauaucgggggcccccugacuaauucaaaagggcagagcugcggcuaucgccggugccgcgccagcggugugcugacgaccagcugcgguaauacccucacauguuacuugaaggcuucugcagccugucgagcugcaaagcuccaggacugcacaauguuagugugcggagacgaccuugucguuaucugugaaagugcgggaacccaggaggacgcggcgagccuacgagucuucacggaggcuaugacuagguacucugccccccccggggacccgccccagccagaauacgacuuggagcugauaacaucaugcuccuccaacgucucggucgcgcacgaugcacuuggcaagcggguguauuaucugacccgcgaccccaccaccccccuugcgcgggcugcgugggagacagcaagacacacuccaguuaacuccuggcuaggcaacaucaucauguaugcgcccacccuaugggcaaggaugauucugaugacccacuucuuuuccauccuucuagcucaggaacaacuugaaaaagcccuagauugucagaucuacggggccacuuacuccauugagccacuugaccuaccucagaucauucagcgacuccacggccuuagcgcauuuucacuccauagcuacucuccaggugagaucaauaggguggcuucaugccucaggaaacuugggguaccacccuugcgagucuggagacaucgggccagaaguguccgcgcuaagcuacugucccagggggggagggccgccacuuguggcaaauaccucuucaacugggcaguaaggaccaagcucaaacucacuccaauuccggcugcgucccaguuggacuuguccggcugguucauugcugguuacagcgggggagacauauaucacagccugucucgcgcccgaccccgcugguucauguggugccuacuccuacuuuccguagggguaggcaucuaucugcuccccaaucgaugaacggggggcuaaacacuccaggccaauaggccauucuguuuuuuuuuuuuuuuuuuuuuuuuuuuuuuuuuuuuuuuuuuuuuuuuuuuuuuccuuuuuuuuuuuuuuuuucccuuucuuuugguggcuccaucuuagcccuagucacggcuagcugugaaagguccgugagccgcaugacugcagagagugcugauacuggccucucugcagaucaugu

SEQ ID NO: 11 - protein, 3010 aa, Hepatitis C virus
MSTNPKPQRKTKRNTNRRPQDVKFPGGGQIVGGVYLLPRRGPRLGVRATRKTSERSQPRERRQPIPKARRPEGRAWAQPGYPWPLYGNEGLGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTVPASAYEVRNVSGTYHVTNDCANSSIVYEAADIIMHTPGCVPCVREGNSSRCWVALTPTLAARNRSIPTTTIRRHVDLLVGAAAFCSAMYVGDLCGSVFLVSQLFTFSPRRYETVQECNCSIYPGHVSGHRMAWDMMMNWSPTAALVVSQLLRIPQAVVDMVAGAHWGVLAGLAYYSMVGNWAKVLIVMLLFAGVDGTTTVTGGAAAFGTRSLASFFTLGPSQKIQLVNTNGSWHINRTALNCNDSLQTGFLAALFYAHKINTSGCPERMASCRPIDKFAQGWGPITHGAPDTSDQRPYCWHYAPRPCGIVPASEVCGPVYCFTPSPVVVGTTDRFGVPTYSWGENKTDVLLLNNTRPPQGNWFGCTWMNGTGFTKTCGGPPCDIGGVGNNTLTCPTDCFRKHPEATYTKCGSGPWLTPRCLVDYPYRLWHYPCTVNFTIFKVRMYVGGVEHRLNAACNWTRGERCDLEDRDRSELSPLLLSTTEWQILPCSFTTLPALSTGLIHLHQNIVDVQYLYGVGSAVVSVVIRWEYVLLLFLLLADARVCACLWMMLLIAQAEAALENLVVLNAASVAGAHGILSFLVFFCAAWYIKGKLVPGVAYALYGVWPLLLLLLALPQRAYAMDREMAASCGGAVFVGLVLLTLSPHYKAFLAKSIWWLQYLITRAEAHLQVWVPPLNVRGGRDAIILLTCAVHPELIFDITKILLAIFGPLMVLQAGLTRVPYFVRAQGLIRVCMLVRKVAGGHYIQMALMRLAALTGTYVYDHLTPLRDWAHAGLRDLAVAVEPVVFSNTETKIITWGADTAACGDIILGLPVSARRGREVLLGPADNFGEQGWRLLAPITAYSQQTRGLLGCIITGLTGRDKNQVEGEVQVVSTATQSFLATCVNGVCWTVYHGAGSKTLAGPKGPITQMYTNVDQDLVGWQAPPGARSMTPCTCGSSDLYLVTRHADVIPVRRRGDSRGSLLSPRPVSYLKGSSGGPLLCPLGHVVGIFRAAVCTRGVAKAVDFIPIESMETTMRSPVFTDNSSPPAVPQTFQVAHLHAPTGSGKSTKVPAAYAAQGYKVLVLNPSVAATLGFGAYMSKAYGVDPNIRTGVRTITTGAPITYSTYGKFLADGGCSGGAYDIIICDECHSTDSTSILGIGTVLDQAETAGARLVVLATATPPGSVTVPHPNIEEVALSNTGEIPFYGKAIPLEVIKGGRHLIFCHSKKKCDELAAKLSGLGVNAVAYYRGLDVSVIPTSGDVVVVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETSVRLRAYLNTPGLPVCQDHLEFWEGVFTGLTHIDAHFLSQTKQAGDNFPYLVAYQATVCARAQAPPPSWDQMWKCLIRLKPTLHGPTPLLYRLGAVQNEVTLTHPMTKYIMTCMSADLEVVTSTWVLVGGVLAALAAYCLTTGSVVIIGRIILSGRPAIIPDREVLYQEFDEMEECASHLPYIEQGMQLAEQFKQKALGLLQTATKQAEAAAPVVESKWQALEAFWAKHMWNFISGIQYLAGLSTLPGNPAIASLMAFTASITSPLTTQHTLLFNILGGWVAAQLAPPSAASAFVGAGIAGAAVGSIGLGKVLVDILAGYGAGVAGALVAFKVMSGEVPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGLPFFSCQRGYKGVWLGDGVMQTTCPCGAQISGHVKNGSMKIVGPKTCSNTWHGTFPINAYTTGPCTPSPAPNYSKALWRVAAEEYVEVTRVGDFHYVTGMTTDNVKCPCQVPAPEFFTELDGVRLHRYAPACKPLLRDEVTFQVGLNQYPVGSQLPCEPEPDVTVLTSMLTDPSHITAETAKRRLARGSPPSLASSSASQLSAPSLKATCTTHHDSPDADLIEANLLWRQEMGGNITRVESENKVVILDSFEPLRAEEDDREVSVAAEILRRTRKFPAAMPIWARPDYNPPLLESWKNPDYVPPVVHGCPLPPIKAPPIPPPRRKRTVILTESTVSSALAELATKTFGSSGSSAVDSGTATGPPDQSSGDGDTGSDAESCSSMPPLEGEPGDPDLSDGSWSTVSEEAGEDVVCCSMSYTWTGALITPCAAEESKLPINALSNSLLRHHNMVYATTSRSAGQRQKKVTFDRVQVLDDHYRDVLKEMKAKASTVKAKLLPVEEACRLTPPHSARSKFGYGAKDVRNLSSKAVNHIHSVWKDLLEDSETPIDTTIMAKNEVFCVQPEKGGRKSARLIVFPDLGVRVCEKMALYDVVSTLPQAVMGSSYGFQYSPGQRVEFLVNAWKSKKNPMGFAYDTRCFDSTVTESDIRVEESIYQCCDLAPEARQVIRSLTERLYIGGPLTNSKGQSCGYRRCRASGVLTTSCGNTLTCYLKASAACRAAKLQDCTMLVCGDDLVVICESAGTQEDAASLRVFTEAMTRYSAPPGDPPQPEYDLELITSCSSNVSVAHDALGKRVYYLTRDPTTPLARAAWETARHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAATCGKYLFNWAVRTKLKLTPIPAASQLDLSGWFIAGYSGGDIYHSLSRARPRWFMWCLLLLSVGVGIYLLPNR

SEQ ID NO: 12 - DNA, 30 bp, Artificial
  primer for HCV
tcatgcggctcacggacctttcacagctag

SEQ ID NO: 13 - DNA, 30 bp, Artificial
  primer for HCV
atcgtcttcacgcagaaagcgtctagccat

SEQ ID NO: 14 - DNA, 26 bp, Artificial
  primer for HCV
gcctattggcctggagtgtttagctc

SEQ ID NO: 15 - DNA, 29 bp, Artificial
  primer for HCV
atggcgttagtatgagtgtcgtgcagcct

SEQ ID NO: 16 - DNA, 31 bp, Artificial
  primer for HCV
tcgggcacgagacaggctgtgatatatgtct

SEQ ID NO: 17 - DNA, 19 bp, Artificial
  primer for HCV
tgcacggtctacgagacct

SEQ ID NO: 18 - DNA, 24 bp, Artificial
  primer for HCV
ctcgcaagcaccctatcagccagt

SEQ ID NO: 19 - DNA, 19 bp, Artificial
  primer for HCV
aggcattgagcgggtttat

SEQ ID NO: 20 - DNA, 38 bp, Artificial
  primer for RACE
ctagactcgagtcgacatcgtttttttttttttttttt

SEQ ID NO: 21 - DNA, 20 bp, Artificial
  primer for HCV
atcttagccctagtcacggc

SEQ ID NO: 22 - DNA, 20 bp, Artificial
  primer for RACE
ctagactcgagtcgacatcg

SEQ ID NO: 23 - DNA, 29 bp, Artificial
  primer for HCV
ctagctgtaaaggtccgtgagccgcatga

SEQ ID NO: 24 - DNA, 17 bp, Artificial
  primer
gtaaaacgacggccagt

SEQ ID NO: 25 - DNA, 17 bp, Artificial
  primer
caggaaacagctatgac

SEQ ID NO: 26 - DNA, 20 bp, Artificial
  primer for HCV
aggaagacttccgagcggtc

SEQ ID NO: 27 - DNA, 32 bp, Artificial
  primer for HCV
ggaacttgcccggttgctctttctctatcttc

SEQ ID NO: 28 - DNA, 25 bp, Artificial
  primer for HCV
attccatggtggggaactgggctaa

SEQ ID NO: 29 - DNA, 30 bp, Artificial
  primer for HCV
taacaataccttgacctgccccacggactg

SEQ ID NO: 30 - DNA, 29 bp, Artificial
  primer for HCV
aacatcgtggacgtgcaatacctgtacgg

SEQ ID NO: 31 - DNA, 20 bp, Artificial
  primer for HCV
gaccctacaccgtacaggta

SEQ ID NO: 32 - DNA, 26 bp, Artificial
  primer for HCV
ttggaccgggagatggctgcatcgtg

SEQ ID NO: 33 - DNA, 21 bp, Artificial
  primer for HCV
cacccaaatgtacaccaatgt

SEQ ID NO: 34 - DNA, 22 bp, Artificial
  primer for HCV
tacccgttgagtctatggaaac

SEQ ID NO: 35 - DNA, 20 bp, Artificial
  primer for HCV
cacttggaatgtctgcggta

SEQ ID NO: 36 - DNA, 25 bp, Artificial
  primer for HCV
agggggggaggcatctcattttctg

SEQ ID NO: 37 - DNA, 26 bp, Artificial
  primer for HCV
tgctatgacgcgggctgtgcttggta

SEQ ID NO: 38 - DNA, 21 bp, Artificial
  primer for HCV
ggtcattgtgggcaggatcat

SEQ ID NO: 39 - DNA, 20 bp, Artificial
  primer for HCV
ctgcctggaaaccccgcgat

SEQ ID NO: 40 - DNA, 24 bp, Artificial
  primer for HCV
tggcagcataggccttgggaaggt

SEQ ID NO: 41 - DNA, 21 bp, Artificial
  primer for HCV
aagacctggctccagtccaag

SEQ ID NO: 42 - DNA, 21 bp, Artificial
  primer for HCV
ttccatgctcaccgacccctc

SEQ ID NO: 43 - DNA, 20 bp, Artificial
  primer for HCV
accttattctctgactccac

SEQ ID NO: 44 - DNA, 21 bp, Artificial
  primer for HCV
cagaagaaggtcacctttgac

SEQ ID NO: 45 - DNA, 25 bp, Artificial
  primer for HCV
gcagcgggtcgagttcctggtgaat

SEQ ID NO: 46 - DNA, 26 bp, Artificial
  primer for HCV
ctacggggcctgttactccattgaac

SEQ ID NO: 47 - DNA, 26 bp, Artificial
  primer for HCV
cttgaaaaagccctggattgtcagat

SEQ ID NO: 48 - DNA, 35 bp, Artificial
  primer for HCV
acatgatctgcagagaggccagtatcagcactctc

SEQ ID NO: 49 - DNA, 31 bp, Artificial
  primer for HCV
gccagccccctgatgggggcgacactccacc

SEQ ID NO: 50 - DNA, 25 bp, Artificial
  primer for HCV
agccgcatgtaagggtatcgatgac

SEQ ID NO: 51 - DNA, 20 bp, Artificial
  primer for HCV
acatgatctgcagagaggcc

SEQ ID NO: 52 - DNA, 30 bp, Artificial
  primer for HCV
gagtacaccggaattgccaggacgaccggg

SEQ ID NO: 53 - DNA, 25 bp, Artificial
  primer for HCV
attccatggtggggaactgggccaa

SEQ ID NO: 54 - DNA, 24 bp, Artificial
  primer for HCV
catccatgtgcagccgaaccaatt

SEQ ID NO: 55 - DNA, 30 bp, Artificial
  primer for HCV
aggggtagtgccaaagcctgtatgggtagt

SEQ ID NO: 56 - DNA, 31 bp, Artificial
  primer for HCV
ataatgacccccggcgactttccgcactaac

SEQ ID NO: 57 - DNA, 20 bp, Artificial
  primer for HCV
catggtagacagtccagcac

SEQ ID NO: 58 - DNA, 24 bp, Artificial
  primer for HCV
gacatgcatgtcatgatgtatttg

SEQ ID NO: 59 - DNA, 20 bp, Artificial
  primer for HCV
ctcatgaccttaaaggccac

SEQ ID NO: 60 - DNA, 30 bp, Artificial
  primer for ECMV IRES
tgcacatgctctacatgtgtttagtcgagg

SEQ ID NO: 61 - RNA, 9594 nt, Hepatitis C virus
gccagcccccugaugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaucaugagcacaaauccuaaaccucaaagaaaaaccaaacguaacaccaaccgccgcccacaggacgucaaguucccgggcgguggucagaucguugguggaguuuaccuguugccgcgcaggggccccagguugggugugcgcgcgacuaggaagacuuccgagcggucgcaaccucgugaaaggcggcaaccuauccccaaggcucgccgccccgagggcagggccugggcucagcccggguacccuuggccccucuauggcaaugagggucuggggugggcaggguggcuccugucaccccgcggcucccggccuaauuggggccccacggacccccggcguaggucgcguaauuuggguaaggucaucgauacccucacaugcggcuucgccgaccucaugggguacauuccgcucgucggcgccccccuaggaggcguugccagggcccuggcgcauggcguccggguucuggaggacggcgugaacuaugcaacagggaauuugcccgguugcucuuucucuaucuuccucuuggcuuugcuguccuguuugacugucccagcuuccgcuuaugaagugcgcaacguguccgggacauaccaugucacgaacgacugcgccaacucaagcauuguguaugaggcagcggacauaaucaugcacaccccugggugcgugcccugcguccgggagggcaauuccucccgcugcuggguagcgcucacucccacgcucgcggccaggaacaggagcauccccacuacgacgauacgacgccacgucgauuugcucguuggggcggcugccuucugcuccgccauguacgugggggaucucugcgggucuguuuuccuagucucccaacuguucaccuucucaccucgccgguaugagacgguacaagaaugcaauugcucaaucuaucccggccacguaucaggucaccgcauggcuugggauaugaugaugaauuggucgcccacagcagcucuaguggugucacaguuacuccgaaucccacaagccgucguggauaugguggcaggggcccacugggggguucuggcgggucuugccuacuauuccaugguggggaacugggcuaaagucuugauugugaugcuacucuuugccggcguugacgggaccaccaccgugacagggggagcggcggccuuuggcaccaggagccuugcguccuucuuuacauuagggccgucucagaaaauccaacuuguaaacaccaacggcaguuggcacaucaauaggacugccuugaauuguaaugacucccuccagacuggguuccuugcugcgcuguucuaugcacacaagaucaacacgucuggaugcccagagcgcauggccagcugccgcccuauugacaaguucgcucagggguggggucccaucacccauggugcgccugacaccucggaccagaggcccuacugcuggcacuacgcaccucgaccgugugguaucguacccgcgucggagguguguggcccaguguauuguuucaccccaagcccuguagugguggggacgaccgaucgcuucggcguccccacguauagcuggggggagaauaagacggacgugcugcuccucaacaacacgcggccgccgcagggcaauugguucggcugcacauggaugaauggcaccggguucaccaagacgugugggggccccccgugugauaucgggggggucggcaacaacaccuugaccugccccacggauugcuuccggaagcaccccgaggccacuuauaccaaaugcggcucggggccuugguugacgccuaggugucuaguugacuacccauacaggcuuuggcacuaccccugcacugucaacuuuaccaucuucaaggucaggauguauguggggggcguggagcacaggcucaacgcugcgugcaacuggacucgaggagagcgcugugaccuagaggacagggacagaucagagcuuagcccgcugcuacugucuacaacagaguggcagauacugcccuguuccuucaccacccuaccggcucuguccacuggcuugauccaccuccaucagaacaucguggacgugcaauaccuguacgguguagggucagcaguugucuccguugugaucagaugggaguacguccugcugcucuuccuccuccuggcggacgcgcgcguuugugccugcuuauggaugaugcuguugauagcccaggcugaagccgccuuggagaaccugguaguccucaacgcggcguccguggccggagcgcauggcauccucucuuuccuuguguucuucugcgcugccugguacaucaaaggcaagcuagugccugggguggcguaugcucucuaugguguauggccgcugcuccugcuccugcuggcguugccacaacgggcguacgccauggaccgggagauggcugcaucgugcggaggcgcgguuuucgugggucugguacucuugaccuugucaccacacuauaaagcauuccuugccaaguccauauggugguuacaauauuuaaucaccagggccgaggcgcauuugcaaguguggguccccccccucaacguucgggggggccgcgaugccaucauccuccucacgugcgcgguccacccagagcuaauuuuugacaucaccaaaauuuugcucgccauauuuggcccgcucauggugcuccaggcuggucuaacuagagugccguacuuugugcgcgcucaagggcucauccgugugugcauguuggugcggaaagucgcugggggucauuauauccaaauggcucucaugagguuggccgcacugacgggcacguacguuuaugaucaucuuacuccgcugcgggacugggcccaugcgggcuugcgagaccuugcgguggcaguugagcccgucguuuucuccaacacagagaccaagauuaucaccuggggggcggacaccgcggcguguggggacaucaucuugggucuaccugucuccgcccgaagggggagagagguacuccuaggaccggccgauaacuuuggagagcaaggguggcgacuccuugcgcccaucacggccuacucccaacagacgcggggcuuacuuggcuguaucaucaccggccucacaggucgagacaagaaccaggucgaaggggagguucaggugguuuccaccgcaacacagucuuucuuggcaaccugcgucaacgguguguguuggacugucuaccauggugccggcucaaagacccuagccggcccgaaggggccaaucacccagauguacaccaacguagaccaagaccucguuggcuggcaggcgccccccggggcgcguuccaugacgccgugcaccugcggcagcucggaccuuuacuuggucacgaggcaugcugaugucauuccggugcgccggcggggugacagcagagggagccuacuuucccccaggcccgucuccuacuugaagggcucuucaggugguccacugcucugccccuuggggcacgucgugggcaucuuucgggcugccgugugcacccgggggguugcgaaggcgguggacuucauacccaucgagucuauggaaacuaccaugcggucuccggucuuuacggauaauucaucucccccggccguaccgcagacuuuucaaguggcccaucugcacgcccccacuggcagcggcaagagcaccaaggugccagcugcguaugcagcccagggguacaaggugcuugucuugaauccauccguugccgccaccuuggguuuuggggcguauauguccaaggcauacggugucgacccuaacauuagaacugggguaaggaccaucaccacaggcgcucccaucacguacuccaccuacggcaaguuccuugccgacggugguugcuccgggggcgcuuacgacaucauaauaugcgaugagugccacucaaccgacucgacuuccauuuugggcauuggcacgguccuggaucaagcggagacggcuggagcgcgacucgucgugcucgccaccgcuacgccuccgggaucggucacugugccacaccccaacaucgaggagguggccuuguccaacaccggagagauucccuucuauggcaaagccaucccccucgaggucaucaagggggggaggcaucucauuuucugucauucuaagaagaagugugaugagcucgcugcaaagcugucgggccuuggggucaacgcuguagcguacuaccggggucuugauguguccgucauaccaacaagcggggacgucguugucguggcaacagacgcucuaaugacgggcuacaccggugacuuugacucugugaucgacuguaauacaugugucacccagacagucgacuucagccuggaccccaccuucaccauugagacgacgaccgugccccaagacgcagugucgcgcucgcagcggcgagggaggacugguagggguagagggggcauauacagguuugugacuccaggagagcggcccucgggcauguucgauuccucgguccugugugaaugcuaugacgcgggcugugcuugguacgagcucacgcccgccgagaccucgguuaggcugcgggcuuaccuaaauacaccaggguugcccgucugccaggaccauuuggaguucugggaaggcgucuucacaggccucacucauauagaugcccacuucuugucucagacuaagcaggcaggagacaacuuccccuaccugguggcauaccaggccacagugugcgccagggcccaggcaccaccuccaucaugggaucaaauguggaagugucucauacggcuaaaaccuacacuacacgggccaacaccccuguuguacaggcuaggagccguccaaaacgaggucacccucacacaccccaugaccaaauacaucaugacaugcaugucggcugaccuagaggucgucaccagcacuugggugcuggugggcgggguccucgcagcuuuggccgcguacugcuugacaacgggcagcgugguuaucauaggcaggaucaucuuguccggaaggccggcuaucauucccgauagggaaguucucuaccaggaguucgaugaaauggaagagugcgccucacaccuucccuacaucgaacaaggaaugcagcucgccgagcaauucaagcagaaggcgcucggguuguugcaaacggccaccaagcaagcggaggccgcugcucccgugguggaguccaaauggcaagcccuugaggccuucugggcgaagcacauguggaacuucaucagcgggauacaguaucuagcaggcuuguccacucugccuggaaaccccgcgauagcaucgcugauggcauuuacagccucuaucacuagcccgcucaccacccuacauacccuucuauuuaacaucuuggggggauggguggccgcccaacucgccccccccagcgcugcuucagccuucguaggcgccggcaucgccggcgcggcuguuggcagcauaggucuugggaaggugcucguggacauccuagcggguuauggagcagggguggcaggcgcacucguggccuucaaggucaugagcggcgaagugcccuccacugaggaccuggucaacuuacucccugccauccucuccccuggugcccuggucgucggggucgugugcgcagcgauacugcgucggcaugugggcccaggggagggggccgugcaguggaugaaccggcugauagcguucgcuucgcgggguaaccacgucucccccacgcacuaugugccugagagcgacgccgcagcgcgugucacccagauccucuccagccuuaccaucacucagcugcuaaagaggcuccaccaguggauuaauaaggacuguuccacgccaugcuccgguucguggcucagggauguuugggacuggauaugcacgguuuugaccgacuucaaaaccuggcuccaguccaagcuccugccacgguugccgggacucccuuucuuuucaugucaacguggauauaaaggagucuggcugggagauggcguuaugcaaacuaccuguccauguggugcacaaaucagcggacaugucaaaaacggcuccaugaagaucguggggccuaaaaccugcagcaacacguggcacgggacguuccccaucaacgcauacaccacaggccccugcacacccuccccggcgccgaacuauuccaaggcguuguggcgaguggcugcugaggaguauguggaggucacgcgggugggggauuuccacuacgugacgggcaugaccacugacaacguaaaaugcccaugccaggucccggcccccgaauucuucacggaguuggauggggugcggcugcacagguacgcuccggcgugcaagccucucuuacgggaugaggucacauuccaggucgggcucaaccaguauccgguuggaucacagcuuccaugugagcccgagccggauguaacagugcucacuuccaugcucaccgaccccucccauauuacagcagagacggcuaagcguaggcuggccagaggguccccccccucuuuggccagcuccucagcuagccaguugucugcgccuuccuugaaggcgacaugcacuacccaccaugacuccccagaugcugaccucaucgaggccaaccuccuguggcggcaggagaugggcgggaacaucacccgcguggagucagagaacaagguaguaauucuggacucuuuugaaccgcuucgagcggaggaggaugauagggaaguguccguagcggcggagauccugcggagaaccaggaaauuccccgcagcgaugcccauaugggcacggccggacuacaacccaccacuccuagagucuuggaagaacccagacuacgucccuccagugguacacgggugcccauuaccaccuauuaaggccccuccgauaccaccuccacggagaaagaggacggucauccugacagaauccaccgugucuucugccuuggcggagcuugcuacaaagaccuuuggcagcuccggaucgucggccguugacagcggcacagcgacuggcccuccugaucaguccuccggugacggagauacaggauccgacgcugagucgugcuccuccaugcccccccuugagggggagccgggggaccccgaucucagcgacgggucuuggucuaccgugagcgaggaggccggugaggacgucgucugcugcucgauguccuacacauggacaggcgccuuaaucacaccgugcgccgcagaggagagcaagcugccuaucaacgcguugagcaacucuuugcugcgccaccacaacauggucuaugccacaacaucccgcagcgcgggccaacggcagaagaaggucacuuuugacagagugcagguccuggacgaccauuaccgggacgugcucaaggagaugaaggcgaaggcguccacaguuaaggcuaaacuucuaccuguagaagaagccugcaggcugacgcccccacacucggccagauccaaguuuggcuauggggcgaaggacguccggaaccuauccagcaaggccgucaaccacauccacuccguguggaaggacuugcuggaagacucugagacgccgauugacaccaccaucauggcaaaaaaugaggucuuuuguguucaaccagagaaaggaggccgcaagucagcucgucuuaucguauucccagacuuggggguucgugugugcgagaaaauggcccuuuacgacgugguuuccacucuuccucaggccgugaugggcuccucauacggguuccaguacucuccuggacagcgggucgaguuccuggugaaugccuggaaaucaaagaagaacccuaugggcuucgcauaugacacccgcuguuuugacucaacggucacugagagugacauccguguugaggagucaauuuaccaauguugugacuuggcccccgaggccagacaggucauaaggucgcucacggagcggcuuuauaucgggggcccccugacuaauucaaaagggcagagcugcggcuaucgccggugccgcgccagcggugugcugacgaccagcugcgguaauacccucacauguuacuugaaggcuucugcagccugucgagcugcaaagcuccaggacugcacaauguuagugugcggagacgaccuugucguuaucugugaaagugcgggaacccaggaggacgcggcgagccuacgagucuucacggaggcuaugacuagguacucugccccccccggggacccgccccagccagaauacgacuuggagcugauaacaucaugcuccuccaacgucucggucgcgcacgaugcacuuggcaagcggguguauuaucugacccgcgaccccaccaccccccuugcgcgggcugcgugggagacagcaagacacacuccaguuaacuccuggcuaggcaacaucaucauguaugcgcccacccuaugggcaaggaugauucugaugacccacuucuuuuccauccuucuagcucaggaacaacuugaaaaagcccuagauugucagaucuacggggccacuuacuccauugagccacuugaccuaccucagaucauucagcgacuccacggccuuagcgcauuuucacuccauagcuacucuccaggugagaucaauaggguggcuucaugccucaggaaacuugggguaccacccuugcgagucuggagacaucgggccagaaguguccgcgcuaagcuacugucccagggggggagggccgccacuuguggcaaauaccucuucaacugggcaguaaggaccaagcucaaacucacuccaauuccggcugcgucccaguuggacuuguccggcugguucauugcugguuacagcgggggagacauauaucacagccugucucgcgcccgaccccgcugguucauguggugccuacuccuacuuuccguagggguaggcaucuaucugcuccccaaucgaugaacggggggcuaaacacuccaggccaauaggccauucuguuuuuuuuuuuuuuuuuuuuuuuuuuuuuuuuuuuuuuuuuuuuuuuuuuuuuuccuuuuuuuuuuuuuuuuucccuuucuuuugguggcuccaucuuagcccuagucacggcuagcugugaaagguccgugagccgcaugacugcagagagugcugauacuggccucucugcagaucaugu

SEQ ID NO: 62 - protein, 3010 aa, Hepatitis C virus
MSTNPKPQRKTKRNTNRRPQDVKFPGGGQIVGGVYLLPRRGPRLGVRATRKTSERSQPRERRQPIPKARRPEGRAWAQPGYPWPLYGNEGLGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTVPASAYEVRNVSGTYHVTNDCANSSIVYEAADIIMHTPGCVPCVREGNSSRCWVALTPTLAARNRSIPTTTIRRHVDLLVGAAAFCSAMYVGDLCGSVFLVSQLFTFSPRRYETVQECNCSIYPGHVSGHRMAWDMMMNWSPTAALVVSQLLRIPQAVVDMVAGAHWGVLAGLAYYSMVGNWAKVLIVMLLFAGVDGTTTVTGGAAAFGTRSLASFFTLGPSQKIQLVNTNGSWHINRTALNCNDSLQTGFLAALFYAHKINTSGCPERMASCRPIDKFAQGWGPITHGAPDTSDQRPYCWHYAPRPCGIVPASEVCGPVYCFTPSPVVVGTTDRFGVPTYSWGENKTDVLLLNNTRPPQGNWFGCTWMNGTGFTKTCGGPPCDIGGVGNNTLTCPTDCFRKHPEATYTKCGSGPWLTPRCLVDYPYRLWHYPCTVNFTIFKVRMYVGGVEHRLNAACNWTRGERCDLEDRDRSELSPLLLSTTEWQILPCSFTTLPALSTGLIHLHQNIVDVQYLYGVGSAVVSVVIRWEYVLLLFLLLADARVCACLWMMLLIAQAEAALENLVVLNAASVAGAHGILSFLVFFCAAWYIKGKLVPGVAYALYGVWPLLLLLLALPQRAYAMDREMAASCGGAVFVGLVLLTLSPHYKAFLAKSIWWLQYLITRAEAHLQVWVPPLNVRGGRDAIILLTCAVHPELIFDITKILLAIFGPLMVLQAGLTRVPYFVRAQGLIRVCMLVRKVAGGHYIQMALMRLAALTGTYVYDHLTPLRDWAHAGLRDLAVAVEPVVFSNTETKIITWGADTAACGDIILGLPVSARRGREVLLGPADNFGEQGWRLLAPITAYSQQTRGLLGCIITGLTGRDKNQVEGEVQVVSTATQSFLATCVNGVCWTVYHGAGSKTLAGPKGPITQMYTNVDQDLVGWQAPPGARSMTPCTCGSSDLYLVTRHADVIPVRRRGDSRGSLLSPRPVSYLKGSSGGPLLCPLGHVVGIFRAAVCTRGVAKAVDFIPIESMETTMRSPVFTDNSSPPAVPQTFQVAHLHAPTGSGKSTKVPAAYAAQGYKVLVLNPSVAATLGFGAYMSKAYGVDPNIRTGVRTITTGAPITYSTYGKFLADGGCSGGAYDIIICDECHSTDSTSILGIGTVLDQAETAGARLVVLATATPPGSVTVPHPNIEEVALSNTGEIPFYGKAIPLEVIKGGRHLIFCHSKKKCDELAAKLSGLGVNAVAYYRGLDVSVIPTSGDVVVVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETSVRLRAYLNTPGLPVCQDHLEFWEGVFTGLTHIDAHFLSQTKQAGDNFPYLVAYQATVCARAQAPPPSWDQMWKCLIRLKPTLHGPTPLLYRLGAVQNEVTLTHPMTKYIMTCMSADLEVVTSTWVLVGGVLAALAAYCLTTGSVVIIGRIILSGRPAIIPDREVLYQEFDEMEECASHLPYIEQGMQLAEQFKQKALGLLQTATKQAEAAAPVVESKWQALEAFWAKHMWNFISGIQYLAGLSTLPGNPAIASLMAFTASITSPLTTLHTLLFNILGGWVAAQLAPPSAASAFVGAGIAGAAVGSIGLGKVLVDILAGYGAGVAGALVAFKVMSGEVPSTEDLVNLLPAILSPGALVVGVVCAAILRRHVGPGEGAVQWMNRLIAFASRGNHVSPTHYVPESDAAARVTQILSSLTITQLLKRLHQWINKDCSTPCSGSWLRDVWDWICTVLTDFKTWLQSKLLPRLPGLPFFSCQRGYKGVWLGDGVMQTTCPCGAQISGHVKNGSMKIVGPKTCSNTWHGTFPINAYTTGPCTPSPAPNYSKALWRVAAEEYVEVTRVGDFHYVTGMTTDNVKCPCQVPAPEFFTELDGVRLHRYAPACKPLLRDEVTFQVGLNQYPVGSQLPCEPEPDVTVLTSMLTDPSHITAETAKRRLARGSPPSLASSSASQLSAPSLKATCTTHHDSPDADLIEANLLWRQEMGGNITRVESENKVVILDSFEPLRAEEDDREVSVAAEILRRTRKFPAAMPIWARPDYNPPLLESWKNPDYVPPVVHGCPLPPIKAPPIPPPRRKRTVILTESTVSSALAELATKTFGSSGSSAVDSGTATGPPDQSSGDGDTGSDAESCSSMPPLEGEPGDPDLSDGSWSTVSEEAGEDVVCCSMSYTWTGALITPCAAEESKLPINALSNSLLRHHNMVYATTSRSAGQRQKKVTFDRVQVLDDHYRDVLKEMKAKASTVKAKLLPVEEACRLTPPHSARSKFGYGAKDVRNLSSKAVNHIHSVWKDLLEDSETPIDTTIMAKNEVFCVQPEKGGRKSARLIVFPDLGVRVCEKMALYDVVSTLPQAVMGSSYGFQYSPGQRVEFLVNAWKSKKNPMGFAYDTRCFDSTVTESDIRVEESIYQCCDLAPEARQVIRSLTERLYIGGPLTNSKGQSCGYRRCRASGVLTTSCGNTLTCYLKASAACRAAKLQDCTMLVCGDDLVVICESAGTQEDAASLRVFTEAMTRYSAPPGDPPQPEYDLELITSCSSNVSVAHDALGKRVYYLTRDPTTPLARAAWETARHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAATCGKYLFNWAVRTKLKLTPIPAASQLDLSGWFIAGYSGGDIYHSLSRARPRWFMWCLLLLSVGVGIYLLPNR

SEQ ID NO: 63 - RNA, 9594 nt, Hepatitis C virus
gccagcccccugaugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaucaugagcacaaauccuaaaccucaaagaaaaaccaaacguaacaccaaccgccgcccacaggacgucaaguucccgggcgguggucagaucguugguggaguuuaccuguugccgcgcaggggccccagguugggugugcgcgcgacuaggaagacuuccgagcggucgcaaccucgugaaaggcggcaaccuauccccaaggcucgccgccccgagggcagggccugggcucagcccggguacccuuggccccucuauggcaaugagggucuggggugggcaggguggcuccugucaccccgcggcucccggccuaauuggggccccacggacccccggcguaggucgcguaauuuggguaaggucaucgauacccucacaugcggcuucgccgaccucaugggguacauuccgcucgucggcgccccccuaggaggcguugccagggcccuggcgcauggcguccggguucuggaggacggcgugaacuaugcaacagggaauuugcccgguugcucuuucucuaucuuccucuuggcuuugcuguccuguuugacugucccagcuuccgcuuaugaagugcgcaacguguccgggacauaccaugucacgaacgacugcgccaacucaagcauuguguaugaggcagcggacauaaucaugcacaccccugggugcgugcccugcguccgggagggcaauuccucccgcugcuggguagcgcucacucccacgcucgcggccaggaacaggagcauccccacuacgacgauacgacgccacgucgauuugcucguuggggcggcugccuucugcuccgccauguacgugggggaucucugcgggucuguuuuccuagucucccaacuguucaccuucucaccucgccgguaugagacgguacaagaaugcaauugcucaaucuaucccggccacguaucaggucaccgcauggcuugggauaugaugaugaauuggucgcccacagcagcucuaguggugucacaguuacuccgaaucccacaagccgucguggauaugguggcaggggcccacugggggguucuggcgggucuugccuacuauuccaugguggggaacugggcuaaagucuugauugugaugcuacucuuugccggcguugacgggaccaccaccgugacagggggagcggcggccuuuggcaccaggagccuugcguccuucuuuacauuagggccgucucagaaaauccaacuuguaaacaccaacggcaguuggcacaucaauaggacugccuugaauuguaaugacucccuccagacuggguuccuugcugcgcuguucuaugcacacaagaucaacacgucuggaugcccagagcgcauggccagcugccgcccuauugacaaguucgcucagggguggggucccaucacccauggugcgccugacaccucggaccagaggcccuacugcuggcacuacgcaccucgaccgugugguaucguacccgcgucggagguguguggcccaguguauuguuucaccccaagcccuguagugguggggacgaccgaucgcuucggcguccccacguauagcuggggggagaauaagacggacgugcugcuccucaacaacacgcggccgccgcagggcaauugguucggcugcacauggaugaauggcaccggguucaccaagacgugugggggccccccgugugauaucgggggggucggcaacaacaccuugaccugccccacggauugcuuccggaagcaccccgaggccacuuauaccaaaugcggcucggggccuugguugacgccuaggugucuaguugacuacccauacaggcuuuggcacuaccccugcacugucaacuuuaccaucuucaaggucaggauguauguggggggcguggagcacaggcucaacgcugcgugcaacuggacucgaggagagcgcugugaccuagaggacagggacagaucagagcuuagcccgcugcuacugucuacaacagaguggcagauacugcccuguuccuucaccacccuaccggcucuguccacuggcuugauccaccuccaucagaacaucguggacgugcaauaccuguacgguguagggucagcaguugucuccguugugaucagaugggaguacguccugcugcucuuccuccuccuggcggacgcgcgcguuugugccugcuuauggaugaugcuguugauagcccaggcugaagccgccuuggagaaccugguaguccucaacgcggcguccguggccggagcgcauggcauccucucuuuccuuguguucuucugcgcugccugguacaucaaaggcaagcuagugccugggguggcguaugcucucuaugguguauggccgcugcuccugcuccugcuggcguugccacaacgggcguacgccauggaccgggagauggcugcaucgugcggaggcgcgguuuucgugggucugguacucuugaccuugucaccacacuauaaagcauuccuugccaaguccauauggugguuacaauauuuaaucaccagggccgaggcgcauuugcaaguguggguccccccccucaacguucgggggggccgcgaugccaucauccuccucacgugcgcgguccacccagagcuaauuuuugacaucaccaaaauuuugcucgccauauuuggcccgcucauggugcuccaggcuggucuaacuagagugccguacuuugugcgcgcucaagggcucauccgugugugcauguuggugcggaaagucgcugggggucauuauauccaaauggcucucaugagguuggccgcacugacgggcacguacguuuaugaucaucuuacuccgcugcgggacugggcccaugcgggcuugcgagaccuugcgguggcaguugagcccgucguuuucuccaacacagagaccaagauuaucaccuggggggcggacaccgcggcguguggggacaucaucuugggucuaccugucuccgcccgaagggggagagagguacuccuaggaccggccgauaacuuuggagagcaaggguggcgacuccuugcgcccaucacggccuacucccaacagacgcggggcuuacuuggcuguaucaucaccggccucacaggucgagacaagaaccaggucgaaggggagguucaggugguuuccaccgcaacacagucuuucuuggcaaccugcgucaacgguguguguuggacugucuaccauggugccggcucaaagacccuagccggcccgaaggggccaaucacccagauguacaccaacguagaccaagaccucguuggcuggcaggcgccccucggggcgcguuccaugacgccgugcaccugcggcagcucggaccuuuacuuggucacgaggcaugcugaugucauuccggugcgccggcggggugacagcagagggagccuacuuucccccaggcccgucuccuacuugaagggcucuucaggugguccacugcucugccccuuggggcacgucgugggcaucuuucgggcugccgugugcacccgggggguugcgaaggcgguggacuucauacccaucgagucuauggaaacuaccaugcggucuccggucuuuacggauaauucaucucccccggccguaccgcagacuuuucaaguggcccaucugcacgcccccacuggcagcggcaagagcaccaaggugccagcugcguaugcagcccagggguacaaggugcuugucuugaauccauccguugccgccaccuuggguuuuggggcguauauguccaaggcauacggugucgacccuaacauuagaacugggguaaggaccaucaccacaggcgcucccaucacguacuccaccuacggcaaguuccuugccgacggugguugcuccgggggcgcuuacgacaucauaauaugcgaugagugccacucaaccgacucgacuuccauuuugggcauuggcacgguccuggaucaagcggagacggcuggagcgcgacucgucgugcucgccaccgcuacgccuccgggaucggucacugugccacaccccaacaucgaggagguggccuuguccaacaccggagagauucccuucuauggcaaagccaucccccucgaggucaucaagggggggaggcaucucauuuucugucauucuaagaagaagugugaugagcucgcugcaaagcugucgggccuuggggucaacgcuguagcguacuaccggggucuugauguguccgucauaccaacaagcggggacgucguugucguggcaacagacgcucuaaugacgggcuacaccggugacuuugacucugugaucgacuguaauacaugugucacccagacagucgacuucagccuggaccccaccuucaccauugagacgacgaccgugccccaagacgcagugucgcgcucgcagcggcgagggaggacugguagggguagagggggcauauacagguuugugacuccaggagagcggcccucgggcauguucgauuccucgguccugugugaaugcuaugacgcgggcugugcuugguacgagcucacgcccgccgagaccucgguuaggcugcgggcuuaccuaaauacaccaggguugcccgucugccaggaccauuuggaguucugggaaggcgucuucacaggccucacucauauagaugcccacuucuugucucagacuaagcaggcaggagacaacuuccccuaccugguggcauaccaggccacagugugcgccagggcccaggcaccaccuccaucaugggaucaaauguggaagugucucauacggcuaaaaccuacacuacacgggccaacaccccuguuguacaggcuaggagccguccaaaacgaggucacccucacacaccccaugaccaaauacaucaugacaugcaugucggcugaccuagaggucgucaccagcacuugggugcuggugggcgggguccucgcagcuuuggccgcguacugcuugacaacgggcagcgugguuaucauaggcaggaucaucuuguccggaaggccggcuaucauucccgauagggaaguucucuaccaggaguucgaugaaauggaagagugcgccucacaccuucccuacaucgaacaaggaaugcagcucgccgagcaauucaagcagaaggcgcucggguuguugcaaacggccaccaagcaagcggaggccgcugcucccgugguggaguccaaauggcaagcccuugaggccuucugggcgaagcacauguggaacuucaucagcgggauacaguaucuagcaggcuuguccacucugccuggaaaccccgcgauagcaucgcugauggcauuuacagccucuaucacuagcccgcucaccacccuacauacccuucuauuuaacaucuuggggggauggguggccgcccaacucgccccccccagcgcugcuucagccuucguaggcgccggcaucgccggcgcggcuguuggcagcauaggucuugggaaggugcucguggacauccuagcggguuauggagcagggguggcaggcgcacucguggccuucaaggucaugagcggcgaagugcccuccacugaggaccuggucaacuuacucccugccauccucuccccuggugcccuggucgucggggucgugugcgcagcgauacugcgucggcaugugggcccaggggagggggccgugcaguggaugaaccggcugauagcguucgcuucgcgggguaaccacgucucccccacgcacuaugugccugagagcgacgccgcagcgcgugucacccagauccucuccagccuuaccaucacucagcugcuaaagaggcuccaccaguggauuaauaaggacuguuccacgccaugcuccgguucguggcucagggauguuugggacuggauaugcacgguuuugaccgacuucaaaaccuggcuccaguccaagcuccugccacgguugccgggacucccuuucuuuucaugucaacguggauauaaaggagucuggcugggagauggcguuaugcaaacuaccuguccauguggugcacaaaucagcggacaugucaaaaacggcuccaugaagaucguggggccuaaaaccugcagcaacacguggcacgggacguuccccaucaacgcauacaccacaggccccugcacacccuccccggcgccgaacuauuccaaggcguuguggcgaguggcugcugaggaguauguggaggucacgcgggugggggauuuccacuacgugacgggcaugaccacugacaacguaaaaugcccaugccaggucccggcccccgaauucuucacggaguuggauggggugcggcugcacagguacgcuccggcgugcaagccucucuuacgggaugaggucacauuccaggucgggcucaaccaguauccgguuggaucacagcuuccaugugagcccgagccggauguaacagugcucacuuccaugcucaccgaccccucccauauuacagcagagacggcuaagcguaggcuggccagaggguccccccccucuuuggccagcuccucagcuagccaguugucugcgccuuccuugaaggcgacaugcacuacccaccaugacuccccagaugcugaccucaucgaggccaaccuccuguggcggcaggagaugggcgggaacaucacccgcguggagucagagaacaagguaguaauucuggacucuuuugaaccgcuucgagcggaggaggaugauagggaaguguccguagcggcggagauccugcggagaaccaggaaauuccccgcagcgaugcccauaugggcacggccggacuacaacccaccacuccuagagucuuggaagaacccagacuacgucccuccagugguacacgggugcccauuaccaccuauuaaggccccuccgauaccaccuccacggagaaagaggacggucauccugacagaauccaccgugucuucugccuuggcggagcuugcuacaaagaccuuuggcagcuccggaucgucggccguugacagcggcacagcgacuggcccuccugaucaguccuccggugacggagauacaggauccgacgcugagucgugcuccuccaugcccccccuugagggggagccgggggaccccgaucucagcgacgggucuuggucuaccgugagcgaggaggccggugaggacgucgucugcugcucgauguccuacacauggacaggcgccuuaaucacaccgugcgccgcagaggagagcaagcugccuaucaacgcguugagcaacucuuugcugcgccaccacaacauggucuaugccacaacaucccgcagcgcgggccaacggcagaagaaggucacuuuugacagagugcagguccuggacgaccauuaccgggacgugcucaaggagaugaaggcgaaggcguccacaguuaaggcuaaacuucuaccuguagaagaagccugcaggcugacgcccccacacucggccagauccaaguuuggcuauggggcgaaggacguccggaaccuauccagcaaggccgucaaccacauccacuccguguggaaggacuugcuggaagacucugagacgccgauugacaccaccaucauggcaaaaaaugaggucuuuuguguucaaccagagaaaggaggccgcaagucagcucgucuuaucguauucccagacuuggggguucgugugugcgagaaaauggcccuuuacgacgugguuuccacucuuccucaggccgugaugggcuccucauacggguuccaguacucuccuggacagcgggucgaguuccuggugaaugccuggaaaucaaagaagaacccuaugggcuucgcauaugacacccgcuguuuugacucaacggucacugagagugacauccguguugaggagucaauuuaccaauguugugacuuggcccccgaggccagacaggucauaaggucgcucacggagcggcuuuauaucgggggcccccugacuaauucaaaagggcagagcugcggcuaucgccggugccgcgccagcggugugcugacgaccagcugcgguaauacccucacauguuacuugaaggcuucugcagccugucgagcugcaaagcuccaggacugcacaauguuagugugcggagacgaccuugucguuaucugugaaagugcgggaacccaggaggacgcggcgagccuacgagucuucacggaggcuaugacuagguacucugccccccccggggacccgccccagccagaauacgacuuggagcugauaacaucaugcuccuccaacgucucggucgcgcacgaugcacuuggcaagcggguguauuaucugacccgcgaccccaccaccccccuugcgcgggcugcgugggagacagcaagacacacuccaguuaacuccuggcuaggcaacaucaucauguaugcgcccacccuaugggcaaggaugauucugaugacccacuucuuuuccauccuucuagcucaggaacaacuugaaaaagcccuagauugucagaucuacggggccacuuacuccauugagccacuugaccuaccucagaucauucagcgacuccacggccuuagcgcauuuucacuccauagcuacucuccaggugagaucaauaggguggcuucaugccucaggaaacuugggguaccacccuugcgagucuggagacaucgggccagaaguguccgcgcuaagcuacugucccagggggggagggccgccacuuguggcaaauaccucuucaacugggcaguaaggaccaagcucaaacucacuccaauuccggcugcgucccaguuggacuuguccggcugguucauugcugguuacagcgggggagacauauaucacagccugucucgcgcccgaccccgcugguucauguggugccuacuccuacuuuccguagggguaggcaucuaucugcuccccaaucgaugaacggggggcuaaacacuccaggccaauaggccauucuguuuuuuuuuuuuuuuuuuuuuuuuuuuuuuuuuuuuuuuuuuuuuuuuuuuuuuccuuuuuuuuuuuuuuuuucccuuucuuuugguggcuccaucuuagcccuagucacggcuagcugugaaagguccgugagccgcaugacugcagagagugcugauacuggccucucugcagaucaugu

SEQ ID NO: 64 - protein, 3010 aa, Hepatitis C virus
MSTNPKPQRKTKRNTNRRPQDVKFPGGGQIVGGVYLLPRRGPRLGVRATRKTSERSQPRERRQPIPKARRPEGRAWAQPGYPWPLYGNEGLGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTVPASAYEVRNVSGTYHVTNDCANSSIVYEAADIIMHTPGCVPCVREGNSSRCWVALTPTLAARNRSIPTTTIRRHVDLLVGAAAFCSAMYVGDLCGSVFLVSQLFTFSPRRYETVQECNCSIYPGHVSGHRMAWDMMMNWSPTAALVVSQLLRIPQAVVDMVAGAHWGVLAGLAYYSMVGNWAKVLIVMLLFAGVDGTTTVTGGAAAFGTRSLASFFTLGPSQKIQLVNTNGSWHINRTALNCNDSLQTGFLAALFYAHKINTSGCPERMASCRPIDKFAQGWGPITHGAPDTSDQRPYCWHYAPRPCGIVPASEVCGPVYCFTPSPVVVGTTDRFGVPTYSWGENKTDVLLLNNTRPPQGNWFGCTWMNGTGFTKTCGGPPCDIGGVGNNTLTCPTDCFRKHPEATYTKCGSGPWLTPRCLVDYPYRLWHYPCTVNFTIFKVRMYVGGVEHRLNAACNWTRGERCDLEDRDRSELSPLLLSTTEWQILPCSFTTLPALSTGLIHLHQNIVDVQYLYGVGSAVVSVVIRWEYVLLLFLLLADARVCACLWMMLLIAQAEAALENLVVLNAASVAGAHGILSFLVFFCAAWYIKGKLVPGVAYALYGVWPLLLLLLALPQRAYAMDREMAASCGGAVFVGLVLLTLSPHYKAFLAKSIWWLQYLITRAEAHLQVWVPPLNVRGGRDAIILLTCAVHPELIFDITKILLAIFGPLMVLQAGLTRVPYFVRAQGLIRVCMLVRKVAGGHYIQMALMRLAALTGTYVYDHLTPLRDWAHAGLRDLAVAVEPVVFSNTETKIITWGADTAACGDIILGLPVSARRGREVLLGPADNFGEQGWRLLAPITAYSQQTRGLLGCIITGLTGRDKNQVEGEVQVVSTATQSFLATCVNGVCWTVYHGAGSKTLAGPKGPITQMYTNVDQDLVGWQAPLGARSMTPCTCGSSDLYLVTRHADVIPVRRRGDSRGSLLSPRPVSYLKGSSGGPLLCPLGHVVGIFRAAVCTRGVAKAVDFIPIESMETTMRSPVFTDNSSPPAVPQTFQVAHLHAPTGSGKSTKVPAAYAAQGYKVLVLNPSVAATLGFGAYMSKAYGVDPNIRTGVRTITTGAPITYSTYGKFLADGGCSGGAYDIIICDECHSTDSTSILGIGTVLDQAETAGARLVVLATATPPGSVTVPHPNIEEVALSNTGEIPFYGKAIPLEVIKGGRHLIFCHSKKKCDELAAKLSGLGVNAVAYYRGLDVSVIPTSGDVVVVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETSVRLRAYLNTPGLPVCQDHLEFWEGVFTGLTHIDAHFLSQTKQAGDNFPYLVAYQATVCARAQAPPPSWDQMWKCLIRLKPTLHGPTPLLYRLGAVQNEVTLTHPMTKYIMTCMSADLEVVTSTWVLVGGVLAALAAYCLTTGSVVIIGRIILSGRPAIIPDREVLYQEFDEMEECASHLPYIEQGMQLAEQFKQKALGLLQTATKQAEAAAPVVESKWQALEAFWAKHMWNFISGIQYLAGLSTLPGNPAIASLMAFTASITSPLTTLHTLLFNILGGWVAAQLAPPSAASAFVGAGIAGAAVGSIGLGKVLVDILAGYGAGVAGALVAFKVMSGEVPSTEDLVNLLPAILSPGALVVGVVCAAILRRHVGPGEGAVQWMNRLIAFASRGNHVSPTHYVPESDAAARVTQILSSLTITQLLKRLHQWINKDCSTPCSGSWLRDVWDWICTVLTDFKTWLQSKLLPRLPGLPFFSCQRGYKGVWLGDGVMQTTCPCGAQISGHVKNGSMKIVGPKTCSNTWHGTFPINAYTTGPCTPSPAPNYSKALWRVAAEEYVEVTRVGDFHYVTGMTTDNVKCPCQVPAPEFFTELDGVRLHRYAPACKPLLRDEVTFQVGLNQYPVGSQLPCEPEPDVTVLTSMLTDPSHITAETAKRRLARGSPPSLASSSASQLSAPSLKATCTTHHDSPDADLIEANLLWRQEMGGNITRVESENKVVILDSFEPLRAEEDDREVSVAAEILRRTRKFPAAMPIWARPDYNPPLLESWKNPDYVPPVVHGCPLPPIKAPPIPPPRRKRTVILTESTVSSALAELATKTFGSSGSSAVDSGTATGPPDQSSGDGDTGSDAESCSSMPPLEGEPGDPDLSDGSWSTVSEEAGEDVVCCSMSYTWTGALITPCAAEESKLPINALSNSLLRHHNMVYATTSRSAGQRQKKVTFDRVQVLDDHYRDVLKEMKAKASTVKAKLLPVEEACRLTPPHSARSKFGYGAKDVRNLSSKAVNHIHSVWKDLLEDSETPIDTTIMAKNEVFCVQPEKGGRKSARLIVFPDLGVRVCEKMALYDVVSTLPQAVMGSSYGFQYSPGQRVEFLVNAWKSKKNPMGFAYDTRCFDSTVTESDIRVEESIYQCCDLAPEARQVIRSLTERLYIGGPLTNSKGQSCGYRRCRASGVLTTSCGNTLTCYLKASAACRAAKLQDCTMLVCGDDLVVICESAGTQEDAASLRVFTEAMTRYSAPPGDPPQPEYDLELITSCSSNVSVAHDALGKRVYYLTRDPTTPLARAAWETARHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAATCGKYLFNWAVRTKLKLTPIPAASQLDLSGWFIAGYSGGDIYHSLSRARPRWFMWCLLLLSVGVGIYLLPNR

SEQ ID NO: 65 - RNA, 783 nt, Hepatitis C virus
gccucacaccuucccuacaucgaacaaggaaugcagcucgccgagcaauucaagcagaaggcgcucggguuguugcaaacggccaccaagcaagcggaggccgcugcucccgugguggaguccaaauggcaagcccuugaggccuucugggcgaagcacauguggaacuucaucagcgggauacaguaucuagcaggcuuguccacucugccuggaaaccccgcgauagcaucgcugauggcauuuacagccucuaucacuagcccgcucaccacccuacauacccuucuauuuaacaucuuggggggauggguggccgcccaacucgccccccccagcgcugcuucagccuucguaggcgccggcaucgccggcgcggcuguuggcagcauaggucuugggaaggugcucguggacauccuagcggguuauggagcagggguggcaggcgcacucguggccuucaaggucaugagcggcgaagugcccuccacugaggaccuggucaacuuacucccugccauccucuccccuggugcccuggucgucggggucgugugcgcagcgauacugcgucggcaugugggcccaggggagggggccgugcaguggaugaaccggcugauagcguucgcuucgcgggguaaccacgucucccccacgcacuaugugccugagagcgacgccgcagcgcgugucacccagauccucuccagccuuaccaucacucagcugcuaaagaggcuccaccaguggauuaauaaggacuguuccacgccaugc

SEQ ID NO: 66 - protein, 261 aa, Hepatitis C virus
ASHLPYIEQGMQLAEQFKQKALGLLQTATKQAEAAAPVVESKWQALEAFWAKHMWNFISGIQYLAGLSTLPGNPAIASLMAFTASITSPLTTLHTLLFNILGGWVAAQLAPPSAASAFVGAGIAGAAVGSIGLGKVLVDILAGYGAGVAGALVAFKVMSGEVPSTEDLVNLLPAILSPGALVVGVVCAAILRRHVGPGEGAVQWMNRLIAFASRGNHVSPTHYVPESDAAARVTQILSSLTITQLLKRLHQWINKDCSTPC

SEQ ID NO: 67 - RNA, 8010 nt, Artificial
  subgenomic HCV RNA replicon
uaauacgacucacuauagccagcccccgauugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaccaugagcacgaauccuaaaccucaaagaaaaaccaaacguaacaccaacgggcgcgccaugauugaacaagauggauugcacgcagguucuccggccgcuuggguggagaggcuauucggcuaugacugggcacaacagacaaucggcugcucugaugccgccguguuccggcugucagcgcaggggcgcccgguucuuuuugucaagaccgaccuguccggugcccugaaugaacugcaggacgaggcagcgcggcuaucguggcuggccacgacgggcguuccuugcgcagcugugcucgacguugucacugaagcgggaagggacuggcugcuauugggcgaagugccggggcaggaucuccugucaucucaccuugcuccugccgagaaaguauccaucauggcugaugcaaugcggcggcugcauacgcuugauccggcuaccugcccauucgaccaccaagcgaaacaucgcaucgagcgagcacguacucggauggaagccggucuugucgaucaggaugaucuggacgaagagcaucaggggcucgcgccagccgaacuguucgccaggcucaaggcgcgcaugcccgacggcgaggaucucgucgugacccauggcgaugccugcuugccgaauaucaugguggaaaauggccgcuuuucuggauucaucgacuguggccggcuggguguggcggaccgcuaucaggacauagcguuggcuacccgugauauugcugaagagcuuggcggcgaaugggcugaccgcuuccucgugcuuuacgguaucgccgcucccgauucgcagcgcaucgccuucuaucgccuucuugacgaguucuucugaguuuaaacagaccacaacgguuucccucuagcgggaucaauuccgccccucucccuccccccccccuaacguuacuggccgaagccgcuuggaauaaggccggugugcguuugucuauauguuauuuuccaccauauugccgucuuuuggcaaugugagggcccggaaaccuuggcccugucuucuugacgagcauuccuaggggucuuuccccucucgccaaaggaaugcaaggucuguuggaugucgugaaggaagcaguuccucugggaagcuucuugaagacaaacaacgucuguagcgacccuuugcaggcagcggaaccccccaccuggcgacaggugccucugcggccaaaagccacguguauaagauacaccugcaaaggcgguacaaccccagugccacguugugaguuggauaguuguggaaagagucaaauggcucuccucaagcguauucaacaaggggcugaaggaugcccagaagguaccccauuguaugggaucugaucuggggccucggugcacaugcucuacauguguuuagucgagguuaaaaaaacgucuaggccccccgaaccacggggacgugguuuuccuuugaaaaacacgauaauaccauggcgcccaucacggccuacucccaacagacgcggggcuuacuuggcuguaucaucaccggccucacaggucgagacaagaaccaggucgaaggggagguucaggugguuuccaccgcaacacagucuuucuuggcaaccugcgucaacgguguguguuggacugucuaccauggugccggcucaaagacccuagccggcccgaaggggccaaucacccagauguacaccaacguagaccaagaccucguuggcuggcaggcgccccccggggcgcguuccaugacgccgugcaccugcggcagcucggaccuuuacuuggucacgaggcaugcugaugucauuccggugcgccggcggggugacagcagagggagccuacuuucccccaggcccgucuccuacuugaagggcucuucaggugguccacugcucugccccuuggggcacgucgugggcaucuuucgggcugccgugugcacccgggggguugcgaaggcgguggacuucauacccaucgagucuauggaaacuaccaugcggucuccggucuuuacggauaauucaucucccccggccguaccgcagacuuuucaaguggcccaucugcacgcccccacuggcagcggcaagagcaccaaggugccagcugcguaugcagcccagggguacaaggugcuugucuugaauccauccguugccgccaccuuggguuuuggggcguauauguccaaggcauacggugucgacccuaacauuagaacugggguaaggaccaucaccacaggcgcucccaucacguacuccaccuacggcaaguuccuugccgacggugguugcuccgggggcgcuuacgacaucauaauaugcgaugagugccacucaaccgacucgacuuccauuuugggcauuggcacgguccuggaucaagcggagacggcuggagcgcgacucgucgugcucgccaccgcuacgccuccgggaucggucacugugccacaccccaacaucgaggagguggccuuguccaacaccggagagauucccuucuauggcaaagccaucccccucgaggucaucaagggggggaggcaucucauuuucugucauucuaagaagaagugugaugagcucgcugcaaagcugucgggccuuggggucaacgcuguagcguacuaccggggucuugauguguccgucauaccaacaagcggggacgucguugucguggcaacagacgcucuaaugacgggcuacaccggugacuuugacucugugaucgacuguaauacaugugucacccagacagucgacuucagccuggaccccaccuucaccauugagacgacgaccgugccccaagacgcagugucgcgcucgcagcggcgagggaggacugguagggguagagggggcauauacagguuugugacuccaggagagcggcccucgggcauguucgauuccucgguccugugugaaugcuaugacgcgggcugugcuugguacgagcucacgcccgccgagaccucgguuaggcugcgggcuuaccuaaauacaccaggguugcccgucugccaggaccauuuggaguucugggaaggcgucuucacaggccucacucauauagaugcccacuucuugucucagacuaagcaggcaggagacaacuuccccuaccugguggcauaccaggccacagugugcgccagggcccaggcaccaccuccaucaugggaucaaauguggaagugucucauacggcuaaaaccuacacuacacgggccaacaccccuguuguacaggcuaggagccguccaaaacgaggucacccucacacaccccaugaccaaauacaucaugacaugcaugucggcugaccuagaggucgucaccagcacuugggugcuggugggcgggguccucgcagcuuuggccgcguacugcuugacaacgggcagcgugguuaucauaggcaggaucaucuuguccggaaggccggcuaucauucccgauagggaaguucucuaccaggaguucgaugaaauggaagagugcgccucacaccuucccuacaucgaacaaggaaugcagcucgccgagcaauucaagcagaaggcgcucggguuguugcaaacggccaccaagcaagcggaggccgcugcucccgugguggaguccaaauggcaagcccuugaggccuucugggcgaagcacauguggaacuucaucagcgggauacaguaucuagcaggcuuguccacucugccuggaaaccccgcgauagcaucgcugauggcauuuacagccucuaucacuagcccgcucaccacccuacauacccuucuauuuaacaucuuggggggauggguggccgcccaacucgccccccccagcgcugcuucagccuucguaggcgccggcaucgccggcgcggcuguuggcagcauaggucuugggaaggugcucguggacauccuagcggguuauggagcagggguggcaggcgcacucguggccuucaaggucaugagcggcgaagugcccuccacugaggaccuggucaacuuacucccugccauccucuccccuggugcccuggucgucggggucgugugcgcagcgauacugcgucggcaugugggcccaggggagggggccgugcaguggaugaaccggcugauagcguucgcuucgcgggguaaccacgucucccccacgcacuaugugccugagagcgacgccgcagcgcgugucacccagauccucuccagccuuaccaucacucagcugcuaaagaggcuccaccaguggauuaauaaggacuguuccacgccaugcuccgguucguggcucagggauguuugggacuggauaugcacgguuuugaccgacuucaaaaccuggcuccaguccaagcuccugccacgguugccgggacucccuuucuuuucaugucaacguggauauaaaggagucuggcugggagauggcguuaugcaaacuaccuguccauguggugcacaaaucagcggacaugucaaaaacggcuccaugaagaucguggggccuaaaaccugcagcaacacguggcacgggacguuccccaucaacgcauacaccacaggccccugcacacccuccccggcgccgaacuauuccaaggcguuguggcgaguggcugcugaggaguauguggaggucacgcgggugggggauuuccacuacgugacgggcaugaccacugacaacguaaaaugcccaugccaggucccggcccccgaauucuucacggaguuggauggggugcggcugcacagguacgcuccggcgugcaagccucucuuacgggaugaggucacauuccaggucgggcucaaccaguauccgguuggaucacagcuuccaugugagcccgagccggauguaacagugcucacuuccaugcucaccgaccccucccauauuacagcagagacggcuaagcguaggcuggccagaggguccccccccucuuuggccagcuccucagcuagccaguugucugcgccuuccuugaaggcgacaugcacuacccaccaugacuccccagaugcugaccucaucgaggccaaccuccuguggcggcaggagaugggcgggaacaucacccgcguggagucagagaacaagguaguaauucuggacucuuuugaaccgcuucgagcggaggaggaugauagggaaguguccguagcggcggagauccugcggagaaccaggaaauuccccgcagcgaugcccauaugggcacggccggacuacaacccaccacuccuagagucuuggaagaacccagacuacgucccuccagugguacacgggugcccauuaccaccuauuaaggccccuccgauaccaccuccacggagaaagaggacggucauccugacagaauccaccgugucuucugccuuggcggagcuugcuacaaagaccuuuggcagcuccggaucgucggccguugacagcggcacagcgacuggcccuccugaucaguccuccggugacggagauacaggauccgacgcugagucgugcuccuccaugcccccccuugagggggagccgggggaccccgaucucagcgacgggucuuggucuaccgugagcgaggaggccggugaggacgucgucugcugcucgauguccuacacauggacaggcgccuuaaucacaccgugcgccgcagaggagagcaagcugccuaucaacgcguugagcaacucuuugcugcgccaccacaacauggucuaugccacaacaucccgcagcgcgggccaacggcagaagaaggucacuuuugacagagugcagguccuggacgaccauuaccgggacgugcucaaggagaugaaggcgaaggcguccacaguuaaggcuaaacuucuaccuguagaagaagccugcaggcugacgcccccacacucggccagauccaaguuuggcuauggggcgaaggacguccggaaccuauccagcaaggccgucaaccacauccacuccguguggaaggacuugcuggaagacucugagacgccgauugacaccaccaucauggcaaaaaaugaggucuuuuguguucaaccagagaaaggaggccgcaagucagcucgucuuaucguauucccagacuuggggguucgugugugcgagaaaauggcccuuuacgacgugguuuccacucuuccucaggccgugaugggcuccucauacggguuccaguacucuccuggacagcgggucgaguuccuggugaaugccuggaaaucaaagaagaacccuaugggcuucgcauaugacacccgcuguuuugacucaacggucacugagagugacauccguguugaggagucaauuuaccaauguugugacuuggcccccgaggccagacaggucauaaggucgcucacggagcggcuuuauaucgggggcccccugacuaauucaaaagggcagagcugcggcuaucgccggugccgcgccagcggugugcugacgaccagcugcgguaauacccucacauguuacuugaaggcuucugcagccugucgagcugcaaagcuccaggacugcacaauguuagugugcggagacgaccuugucguuaucugugaaagugcgggaacccaggaggacgcggcgagccuacgagucuucacggaggcuaugacuagguacucugccccccccggggacccgccccagccagaauacgacuuggagcugauaacaucaugcuccuccaacgucucggucgcgcacgaugcacuuggcaagcggguguauuaucugacccgcgaccccaccaccccccuugcgcgggcugcgugggagacagcaagacacacuccaguuaacuccuggcuaggcaacaucaucauguaugcgcccacccuaugggcaaggaugauucugaugacccacuucuuuuccauccuucuagcucaggaacaacuugaaaaagcccuagauugucagaucuacggggccacuuacuccauugagccacuugaccuaccucagaucauucagcgacuccacggccuuagcgcauuuucacuccauagcuacucuccaggugagaucaauaggguggcuucaugccucaggaaacuugggguaccacccuugcgagucuggagacaucgggccagaaguguccgcgcuaagcuacugucccagggggggagggccgccacuuguggcaaauaccucuucaacugggcaguaaggaccaagcucaaacucacuccaauuccggcugcgucccaguuggacuuguccggcugguucauugcugguuacagcgggggagacauauaucacagccugucucgcgcccgaccccgcugguucauguggugccuacuccuacuuuccguagggguaggcaucuaucugcuccccaaucgaugaacggggggcuaaacacuccaggccaauaggccauucuguuuuuuuuuuuuuuuuuuuuuuuuuuuuuuuuuuuuuuuuuuuuuuuuuuuuuuccuuuuuuuuuuuuuuuuucccuuucuuuugguggcuccaucuuagcccuagucacggcuagcugugaaagguccgugagccgcaugacugcagagagugcugauacuggccucucugcagaucaugu

SEQ ID NO: 68 - RNA, 8010 nt, Artificial
  subgenomic HCV RNA replicon
uaauacgacucacuauagccagcccccgauugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcaccaugagcacgaauccuaaaccucaaagaaaaaccaaacguaacaccaacgggcgcgccaugauugaacaagauggauugcacgcagguucuccggccgcuuggguggagaggcuauucggcuaugacugggcacaacagacaaucggcugcucugaugccgccguguuccggcugucagcgcaggggcgcccgguucuuuuugucaagaccgaccuguccggugcccugaaugaacugcaggacgaggcagcgcggcuaucguggcuggccacgacgggcguuccuugcgcagcugugcucgacguugucacugaagcgggaagggacuggcugcuauugggcgaagugccggggcaggaucuccugucaucucaccuugcuccugccgagaaaguauccaucauggcugaugcaaugcggcggcugcauacgcuugauccggcuaccugcccauucgaccaccaagcgaaacaucgcaucgagcgagcacguacucggauggaagccggucuugucgaucaggaugaucuggacgaagagcaucaggggcucgcgccagccgaacuguucgccaggcucaaggcgcgcaugcccgacggcgaggaucucgucgugacccauggcgaugccugcuugccgaauaucaugguggaaaauggccgcuuuucuggauucaucgacuguggccggcuggguguggcggaccgcuaucaggacauagcguuggcuacccgugauauugcugaagagcuuggcggcgaaugggcugaccgcuuccucgugcuuuacgguaucgccgcucccgauucgcagcgcaucgccuucuaucgccuucuugacgaguucuucugaguuuaaacagaccacaacgguuucccucuagcgggaucaauuccgccccucucccuccccccccccuaacguuacuggccgaagccgcuuggaauaaggccggugugcguuugucuauauguuauuuuccaccauauugccgucuuuuggcaaugugagggcccggaaaccuuggcccugucuucuugacgagcauuccuaggggucuuuccccucucgccaaaggaaugcaaggucuguuggaugucgugaaggaagcaguuccucugggaagcuucuugaagacaaacaacgucuguagcgacccuuugcaggcagcggaaccccccaccuggcgacaggugccucugcggccaaaagccacguguauaagauacaccugcaaaggcgguacaaccccagugccacguugugaguuggauaguuguggaaagagucaaauggcucuccucaagcguauucaacaaggggcugaaggaugcccagaagguaccccauuguaugggaucugaucuggggccucggugcacaugcucuacauguguuuagucgagguuaaaaaaacgucuaggccccccgaaccacggggacgugguuuuccuuugaaaaacacgauaauaccauggcgcccaucacggccuacucccaacagacgcggggcuuacuuggcuguaucaucaccggccucacaggucgagacaagaaccaggucgaaggggagguucaggugguuuccaccgcaacacagucuuucuuggcaaccugcgucaacgguguguguuggacugucuaccauggugccggcucaaagacccuagccggcccgaaggggccaaucacccagauguacaccaacguagaccaagaccucguuggcuggcaggcgccccucggggcgcguuccaugacgccgugcaccugcggcagcucggaccuuuacuuggucacgaggcaugcugaugucauuccggugcgccggcggggugacagcagagggagccuacuuucccccaggcccgucuccuacuugaagggcucuucaggugguccacugcucugccccuuggggcacgucgugggcaucuuucgggcugccgugugcacccgggggguugcgaaggcgguggacuucauacccaucgagucuauggaaacuaccaugcggucuccggucuuuacggauaauucaucucccccggccguaccgcagacuuuucaaguggcccaucugcacgcccccacuggcagcggcaagagcaccaaggugccagcugcguaugcagcccagggguacaaggugcuugucuugaauccauccguugccgccaccuuggguuuuggggcguauauguccaaggcauacggugucgacccuaacauuagaacugggguaaggaccaucaccacaggcgcucccaucacguacuccaccuacggcaaguuccuugccgacggugguugcuccgggggcgcuuacgacaucauaauaugcgaugagugccacucaaccgacucgacuuccauuuugggcauuggcacgguccuggaucaagcggagacggcuggagcgcgacucgucgugcucgccaccgcuacgccuccgggaucggucacugugccacaccccaacaucgaggagguggccuuguccaacaccggagagauucccuucuauggcaaagccaucccccucgaggucaucaagggggggaggcaucucauuuucugucauucuaagaagaagugugaugagcucgcugcaaagcugucgggccuuggggucaacgcuguagcguacuaccggggucuugauguguccgucauaccaacaagcggggacgucguugucguggcaacagacgcucuaaugacgggcuacaccggugacuuugacucugugaucgacuguaauacaugugucacccagacagucgacuucagccuggaccccaccuucaccauugagacgacgaccgugccccaagacgcagugucgcgcucgcagcggcgagggaggacugguagggguagagggggcauauacagguuugugacuccaggagagcggcccucgggcauguucgauuccucgguccugugugaaugcuaugacgcgggcugugcuugguacgagcucacgcccgccgagaccucgguuaggcugcgggcuuaccuaaauacaccaggguugcccgucugccaggaccauuuggaguucugggaaggcgucuucacaggccucacucauauagaugcccacuucuugucucagacuaagcaggcaggagacaacuuccccuaccugguggcauaccaggccacagugugcgccagggcccaggcaccaccuccaucaugggaucaaauguggaagugucucauacggcuaaaaccuacacuacacgggccaacaccccuguuguacaggcuaggagccguccaaaacgaggucacccucacacaccccaugaccaaauacaucaugacaugcaugucggcugaccuagaggucgucaccagcacuugggugcuggugggcgggguccucgcagcuuuggccgcguacugcuugacaacgggcagcgugguuaucauaggcaggaucaucuuguccggaaggccggcuaucauucccgauagggaaguucucuaccaggaguucgaugaaauggaagagugcgccucacaccuucccuacaucgaacaaggaaugcagcucgccgagcaauucaagcagaaggcgcucggguuguugcaaacggccaccaagcaagcggaggccgcugcucccgugguggaguccaaauggcaagcccuugaggccuucugggcgaagcacauguggaacuucaucagcgggauacaguaucuagcaggcuuguccacucugccuggaaaccccgcgauagcaucgcugauggcauuuacagccucuaucacuagcccgcucaccacccuacauacccuucuauuuaacaucuuggggggauggguggccgcccaacucgccccccccagcgcugcuucagccuucguaggcgccggcaucgccggcgcggcuguuggcagcauaggucuugggaaggugcucguggacauccuagcggguuauggagcagggguggcaggcgcacucguggccuucaaggucaugagcggcgaagugcccuccacugaggaccuggucaacuuacucccugccauccucuccccuggugcccuggucgucggggucgugugcgcagcgauacugcgucggcaugugggcccaggggagggggccgugcaguggaugaaccggcugauagcguucgcuucgcgggguaaccacgucucccccacgcacuaugugccugagagcgacgccgcagcgcgugucacccagauccucuccagccuuaccaucacucagcugcuaaagaggcuccaccaguggauuaauaaggacuguuccacgccaugcuccgguucguggcucagggauguuugggacuggauaugcacgguuuugaccgacuucaaaaccuggcuccaguccaagcuccugccacgguugccgggacucccuuucuuuucaugucaacguggauauaaaggagucuggcugggagauggcguuaugcaaacuaccuguccauguggugcacaaaucagcggacaugucaaaaacggcuccaugaagaucguggggccuaaaaccugcagcaacacguggcacgggacguuccccaucaacgcauacaccacaggccccugcacacccuccccggcgccgaacuauuccaaggcguuguggcgaguggcugcugaggaguauguggaggucacgcgggugggggauuuccacuacgugacgggcaugaccacugacaacguaaaaugcccaugccaggucccggcccccgaauucuucacggaguuggauggggugcggcugcacagguacgcuccggcgugcaagccucucuuacgggaugaggucacauuccaggucgggcucaaccaguauccgguuggaucacagcuuccaugugagcccgagccggauguaacagugcucacuuccaugcucaccgaccccucccauauuacagcagagacggcuaagcguaggcuggccagaggguccccccccucuuuggccagcuccucagcuagccaguugucugcgccuuccuugaaggcgacaugcacuacccaccaugacuccccagaugcugaccucaucgaggccaaccuccuguggcggcaggagaugggcgggaacaucacccgcguggagucagagaacaagguaguaauucuggacucuuuugaaccgcuucgagcggaggaggaugauagggaaguguccguagcggcggagauccugcggagaaccaggaaauuccccgcagcgaugcccauaugggcacggccggacuacaacccaccacuccuagagucuuggaagaacccagacuacgucccuccagugguacacgggugcccauuaccaccuauuaaggccccuccgauaccaccuccacggagaaagaggacggucauccugacagaauccaccgugucuucugccuuggcggagcuugcuacaaagaccuuuggcagcuccggaucgucggccguugacagcggcacagcgacuggcccuccugaucaguccuccggugacggagauacaggauccgacgcugagucgugcuccuccaugcccccccuugagggggagccgggggaccccgaucucagcgacgggucuuggucuaccgugagcgaggaggccggugaggacgucgucugcugcucgauguccuacacauggacaggcgccuuaaucacaccgugcgccgcagaggagagcaagcugccuaucaacgcguugagcaacucuuugcugcgccaccacaacauggucuaugccacaacaucccgcagcgcgggccaacggcagaagaaggucacuuuugacagagugcagguccuggacgaccauuaccgggacgugcucaaggagaugaaggcgaaggcguccacaguuaaggcuaaacuucuaccuguagaagaagccugcaggcugacgcccccacacucggccagauccaaguuuggcuauggggcgaaggacguccggaaccuauccagcaaggccgucaaccacauccacuccguguggaaggacuugcuggaagacucugagacgccgauugacaccaccaucauggcaaaaaaugaggucuuuuguguucaaccagagaaaggaggccgcaagucagcucgucuuaucguauucccagacuuggggguucgugugugcgagaaaauggcccuuuacgacgugguuuccacucuuccucaggccgugaugggcuccucauacggguuccaguacucuccuggacagcgggucgaguuccuggugaaugccuggaaaucaaagaagaacccuaugggcuucgcauaugacacccgcuguuuugacucaacggucacugagagugacauccguguugaggagucaauuuaccaauguugugacuuggcccccgaggccagacaggucauaaggucgcucacggagcggcuuuauaucgggggcccccugacuaauucaaaagggcagagcugcggcuaucgccggugccgcgccagcggugugcugacgaccagcugcgguaauacccucacauguuacuugaaggcuucugcagccugucgagcugcaaagcuccaggacugcacaauguuagugugcggagacgaccuugucguuaucugugaaagugcgggaacccaggaggacgcggcgagccuacgagucuucacggaggcuaugacuagguacucugccccccccggggacccgccccagccagaauacgacuuggagcugauaacaucaugcuccuccaacgucucggucgcgcacgaugcacuuggcaagcggguguauuaucugacccgcgaccccaccaccccccuugcgcgggcugcgugggagacagcaagacacacuccaguuaacuccuggcuaggcaacaucaucauguaugcgcccacccuaugggcaaggaugauucugaugacccacuucuuuuccauccuucuagcucaggaacaacuugaaaaagcccuagauugucagaucuacggggccacuuacuccauugagccacuugaccuaccucagaucauucagcgacuccacggccuuagcgcauuuucacuccauagcuacucuccaggugagaucaauaggguggcuucaugccucaggaaacuugggguaccacccuugcgagucuggagacaucgggccagaaguguccgcgcuaagcuacugucccagggggggagggccgccacuuguggcaaauaccucuucaacugggcaguaaggaccaagcucaaacucacuccaauuccggcugcgucccaguuggacuuguccggcugguucauugcugguuacagcgggggagacauauaucacagccugucucgcgcccgaccccgcugguucauguggugccuacuccuacuuuccguagggguaggcaucuaucugcuccccaaucgaugaacggggggcuaaacacuccaggccaauaggccauucuguuuuuuuuuuuuuuuuuuuuuuuuuuuuuuuuuuuuuuuuuuuuuuuuuuuuuuccuuuuuuuuuuuuuuuuucccuuucuuuugguggcuccaucuuagcccuagucacggcuagcugugaaagguccgugagccgcaugacugcagagagugcugauacuggccucucugcagaucaugu